(19) United States
(12) Patent Application Publication
(10) Pub. No.: US 2025/0066855 A1
(43) Pub. Date: Feb. 27, 2025

(54) DETECTION OF GENETIC VARIANTS OF THE BETA-ADRENERGIC RECEPTOR PATHWAY FOR DETECTING AND TREATING RETINOPATHY OF PREMATURITY

(71) Applicants: William Vance GOOD, Kentfield, CA (US); Robert GENDRON, Logy Bay (CA); Helene PARADIS, Logy Bay (CA); Margaret Quine McGOVERN, San Francisco, CA (US)

(72) Inventors: William Vance GOOD, Kentfield, CA (US); Robert GENDRON, Logy Bay (CA); Helene PARADIS, Logy Bay (CA); Margaret Quine McGOVERN, San Francisco, CA (US)

(21) Appl. No.: 18/814,321

(22) Filed: Aug. 23, 2024

Related U.S. Application Data
(60) Provisional application No. 63/534,536, filed on Aug. 24, 2023.

(51) Int. Cl. C12Q 1/6883; A61K 45/06; C12N 9/22; C12N 15/113; C12Q 1/6806; C12Q 1/6855; C12Q 1/686; C12Q 1/6874

(52) U.S. Cl. CPC C12Q 1/6883; A61K 45/06; C12N 9/22; C12N 15/113; C12Q 1/6806; C12Q 1/6855; C12Q 1/686; C12Q 1/6874; C12N 2310/11; C12N 2310/14; C12N 2310/531; C12Q 2600/106; C12Q 2600/156

(57) ABSTRACT

Described herein are methods for the identification and detection of genetic factors in the molecular pathways associated with vascular retinopathies or retinal diseases, for example mild, moderate or severe retinopathy of prematurity (ROP). Included herewith are methods of detecting genetic mutations associated with severe retinopathies, including factors in ADRβ2 pathways, which include RAPGEF3, ADCY7, ADCY9, PRKARIA, and ADCY4 variants. Further, this disclosure provides for methods of treating a subject having a detected genetic factor indicative of ROP.

### RELATED APPLICATIONS

This disclosure claims priority to U.S. Provisional Application No. 63/534,536, filed Aug. 24, 2023, the contents of which are herein incorporated by reference in their entirety.

### SEQUENCE STATEMENT

The instant application contains a Sequence Listing, which has been submitted electronically and is hereby incorporated by reference in its entirety. Said file is named R4133-00101_SEQ_ID_LISTING.xml and is 2,859 bytes in size.

### FIELD

This disclosure relates to the identification and detection of genetic factors in the molecular pathways associated with vascular retinopathies or retinal diseases, for example mild, moderate or severe retinopathy of prematurity (ROP). In particular, this invention relates to detecting genetic associated with severe retinopathies, including factors in ADRβ2 pathways, which include RAPGEF3, ADCY7, ADCY9, PRKARIA, and ADCY4 variants. Further, this disclosure relates to methods of treating a subject having a detected genetic factor indicative of ROP.

### BACKGROUND

The following includes information that may be useful in understanding the present invention. It is not an admission that any of the information, publications or documents specifically or implicitly referenced herein is prior art, or essential, to the presently described or claimed inventions. All publications, patents, related applications, and other written or electronic materials mentioned or identified herein are hereby incorporated herein by reference in their entirety. The information incorporated is as much a part of the application as filed as if all of the text and other content was repeated in the application, and should be treated as part of the text and content of the application as filed.

Proliferative retinopathies are characterized by excessive pre-retinal blood vessel growth (e.g., neovascularization) that can result in fibrous scar formation and retinal detachment, and are a leading cause of blindness. Proliferative retinopathies include retinopathy of prematurity (ROP), proliferative diabetic retinopathy (PDR), a retinal vein occlusion retinopathy (RVO), hypertensive retinopathy, branch retinal vein occlusion (BRVO), central retinal artery occlusion (CRAO), central retinal vein occlusion, chronic BRVO, Coats' Disease, cotton-wool spots, dot and blot hemorrhage, flame-shaped hemorrhage, hard exudates, hollenhorst plaques, inferior hemicentral retinal vein occlusion, juxtafoveal telangiectasia, optociliary shunt, preretinal hemorrhage, retinal artery macroaneurysm, sickle proliferative retinopathy, subretinal hemorrhage, superior hemicentral retinal vein occlusion, fibrovascular proliferation in PDR, neovascularization of the disc, nonproliferative diabetic retinopathy (NPDR), panretinal photocoagulation, retinal vessel occlusive disease, macular degeneration including age-related macular degeneration, and other retinopathies stemming from retinal vein occlusion.

Retinopathy of prematurity (ROP) is a rare neovascular eye disease of some premature infants. The vascular changes which occur in ROP may lead to total retinal detachment and several visual impairment or blindness (Jefferies, A L. Paediatr Child Health. 15(10): 667-670 (2010)). In ROP, developing retinal blood vessels that are under the influence of a range of angiogenic factors can form new abnormally proliferative and diaphanous vessels that leak protein and other factors, potentially causing cicatricial changes, and ultimately, leading to retinal detachment. The result is partial or total permanent blindness in some infants. Even though there are effective treatments to prevent retinal detachment in ROP, some infants nevertheless are blinded by the disease, and the rate of complications from ROP, including myopia, amblyopia, strabismus, cataract, and glaucoma are high (Bremer D L, Rogers D L, Good W V, Tung B, Hardy R H, Fellow R. Glaucoma in the early treatment for retinopathy of prematurity study. JAAPOS. 16:449-452 (2012)). Most infants with successfully treated ROP fail to develop 20/20 visual acuity (Good W V, Hardy R J. The multicenter study of Early Treatment for Retinopathy of Prematurity (ETROP). Ophthalmology. 108(6):1013-1014 (2001)). Not all preterm infants develop ROP, or develop severe ROP (Type 1) that leads to devastating consequences. Many risk factors for developing ROP have been identified. Low birthweight, young gestational age and prolonged oxygenation at birth are the most prominent risk factors for ROP (Kim S J, et al. Retinopathy of prematurity: a review of risk factors and their clinical significance. Surv Ophthalmol. 63(5):618-637 (2018)). However, the presence of these factors in subjects does not always correlate with the development of severe ROP.

Two large studies have shown that heredity plays a significant role in ROP indicating that genetic factors are likely responsible for causing ROP (Bizzarro et al. Genetic susceptibility to retinopathy of prematurity. Pediatrics. 118(5):1858-1863 (2006); Ortega-Molina et al. Genetic and Environmental Influences on Retinopathy of Prematurity. Mediators Inflamm. 2015:764159 (2015)). Ethnicity and race have been shown to play a role in ROP (Kim S J, et al. Retinopathy of prematurity: a review of risk factors and their clinical significance. Surv Ophthalmol. 63(5):618-637 (2018)). African-American infants develop severe ROP at a rate that is approximately 50% lower than their Caucasian counterparts (Good et al. Early Treatment for Retinopathy of Prematurity Cooperative Group. The incidence and course of retinopathy of prematurity: findings from the early treatment for retinopathy of prematurity study. Pediatrics. 116(1):15-23 (2005)). Many studies have evaluated possible association of various candidate genes with ROP but have failed to identify a strong link with specific genetic variations (Kim S J, et al. Retinopathy of prematurity: a review of risk factors and their clinical significance. Surv Ophthalmol. 63(5):618-637 (2018). Swan et al. Imaging and Informatics in ROP Research Consortium. The genetics of retinopathy of prematurity: a model for neovascular retinal disease. Ophthalmol Retina. Sep; 2(9):949-962 (2018)). Several small cohort studies of genes involved in angiogenic signals such as the vascular endothelial growth factor (VEGF) and Wnt signaling pathways or regulating vascular function such as endothelial nitric oxide synthase, have produced contradictory results or failed to reveal a clear association with ROP. A larger cohort genetic study identified two variants located in an intron of the brain-derived neurotrophic factor (BDNF) gene as significantly linked to ROP which merits further investigation (Hartnett et al. Genomics Subcommittee. Genetic variants associated with severe retinopathy of prematurity in extremely low birth weight infants. Invest Ophthalmol Vis Sci. 55(10):6194-6203 (2014)). A recent whole exome analyses failed to reveal a genetic association with ROP possibly due to the insufficient cohort size (Kim, S J., et al. Imaging and Informatics in Retinopathy of Prematurity (i-ROP) Research Consortium. Identification of candidate genes and pathways in retinopathy of prematurity by whole exome sequencing of preterm infants enriched in phenotypic extremes. Sci Rep. 11(1):4966 (2021)). Genome-wide association studies require several thousands of specimens which are difficult to obtain in the case of rare diseases such as ROP in which many different confounding risk factors might impact outcome (Ehret, GB. Genome-wide association studies: Contribution of genomics to understanding blood pressure and essential hypertension. Curr Hypertens Rep. 12(1):17-25 (2010)).

### SUMMARY

In one aspect, this disclosure relates to methods for detecting genetic factors in molecular pathways associated with vascular retinopathies including, e.g., any neovascular retinal disease. In some aspects, the genetic factors are detected in genes of the beta adrenergic receptor (ADRβ) pathway, including beta-1, beta-2, and beta-3 adrenergic receptors.

In some aspects, this disclosure provides for a method of detecting one or more gene variants in a sample from a subject having or suspected of having a vascular retinopathy, the method comprising: obtaining or having obtained a sample comprising free nucleic acids from a subject; attaching adapters to the free nucleic acids or modified nucleic acids derived therefrom to produce adapter-nucleic acids; optionally amplifying the adapter-nucleic acids to generate adapter-nucleic acid amplicons; and detecting whether gene variants are present in the amplicons. In some aspects, detecting whether gene variants are present in the amplicons comprises the steps: (i) contacting the amplicons with a flow cell or solid surface comprising a plurality of nucleotides connected thereto to generate immobilized amplicons, wherein the oligonucleotides bind to the adapter, (ii), clonally amplifying the immobilized amplicons to generate clonally amplified amplicons, and (iii) sequencing all of or at least a portion of the sequences of the clonally amplified gene amplicons. In another aspect, the method of detecting gene variants further comprises sequencing at least a portion of the adapter-nucleic acids and/or amplicons by massively parallel sequencing. In one aspect, massively parallel sequencing is selected from targeted sequencing or random sequencing. In one aspect, massively parallel sequencing includes single molecule sequencing.

In some aspects, the genetic factors detected in the ADRβ pathway include, but are not limited to, variants in one or more genes selected from the group of genes consisting of RAPGEF3, ADCY4, ADCY7, ADCY9, and PRKAR1A.

In some aspects, the gene variants detected in the ADRβ pathway include or exclude one or more of rs8082254, rs72847785, rs3181254, rs17256902, rs2240079, rs61917617, rs11168215, rs11168214, rs55683248, rs2072341.

In one aspect, this disclosure provides for a method of detecting vascular retinopathy by detecting biomarkers. The biomarkers can include those involved in the ADRβ pathway. In some aspects, the ADRβ pathway-involved biomarkers can include or exclude: rs8082254, rs72847785, rs3181254, rs17256902, rs2240079, rs61917617, rs11168215, rs11168214, rs55683248, rs2072341. In some aspects, this disclosure provides for a method of detecting a vascular retinopathy by detecting biomarkers such as genetic variants in the ADRβ pathway.

The methods of this disclosure can identify and detect genetic factors in the molecular pathways associated with vascular retinopathies or retinal diseases such as mild, moderate, or servere ROP. In some aspects, the retinopathy is selected from retinopathy of prematurity (ROP), proliferative diabetic retinopathy (PDR), a retinal vein occlusion retinopathy (RVO), hypertensive retinopathy, branch retinal vein occlusion (BRVO), central retinal artery occlusion (CRAO), central retinal vein occlusion, chronic BRVO, Coats' Disease, cotton-wool spots, dot and blot hemorrhage, flame-shaped hemorrhage, hard exudates, hollenhorst plaques, inferior hemicentral retinal vein occlusion, juxtafoveal telangiectasia, optociliary shunt, preretinal hemorrhage, retinal artery macroaneurysm, sickle proliferative retinopathy, subretinal hemorrhage, superior hemicentral retinal vein occlusion, fibrovascular proliferation in PDR, neovascularization of the disc, nonproliferative diabetic retinopathy (NPDR), panretinal photocoagulation, retinal vessel occlusive disease, macular degeneration including age-related macular degeneration, or any neovascular retinal disease. In some aspects, the disease is ROP.

In some aspects, this disclosure provides for methods for preparing amplicons or enriched nucleic acid samples from a subject having or suspected of having a vascular retinopathy, the method comprising obtaining or having obtained a sample comprising free nucleic acids from a subject, attaching adapters to the free nucleic acids of nucleic acids derived thereforem to produce adapter-nucleic acids, and amplifying the adapter-nucleic acids to generate amplicons. In some aspects, the methods for preparing amplicons or enriched nucleic acid samples further comprises the steps of: (i) contacting the amplicons with a flow cell or solid surface comprising a plurality of nucleotides connected thereto to generate immobilized amplicons, wherein the oligonucleotides bind to the adapter, (ii), clonally amplifying the immobilized amplicons to generate clonally amplified amplicons, and (iii) sequencing all of or at least a portion of the sequences of the clonally amplified gene amplicons.

In some aspects, the free nucleic acids or nucleic acids derived therefrom may be contacted with other enzymes which can include or exclude the singular or plural of: restriction enzymes, endonucleases, exonucleases, transposons, polymerases, kinases, helicases, and ligases, in preparation for adding adapters. In some aspects, the free nucleic acids or nucleic acids derived therefrom may be contacted with T4 DNA polymerase, T4 polynucleotide kinase, Taq DNA polymerase, DNA polymerase I Large (Klenow) fragment, and/or T4 DNA ligase. In some aspects, adding adapters may comprise a single step or a multi-step process.

In some aspects, RAPGEF3 gene amplicons are obtained by using a forward primer having 80% homology or higher to that of the nucleotide sequence 5′ CTTCCTTCATTTCTCCACCTG 3′ (SEQ ID NO: 1) and the reverse primer having 80% homology or higher to that of the nucleotide sequence 5′ TCTGTGTCCTCTTGCCTGC 3′ (SEQ ID NO: 2). In some aspects, RAPGEF3 gene amplicons are obtained by using a forward primer having 1, 2, 3, 4 or 5 nucleotide substitutions, insertions, or deletions, from that of SEQ ID NO: 1. In some aspects, RAPGEF3 gene amplicons are obtained by using a forward primer having 1, 2, 3, 4 or 5 nucleotide substitutions, insertions, or deletions, from that of SEQ ID NO: 1, and a Tₘ of within 80% to 120% that of the calculated (using NCBI BLAST method) Tₘ for SEQ ID NO: 1. In some aspects, RAPGEF3 gene amplicons are obtained by using a reverse primer having 1, 2, 3, 4 or 5 nucleotide substitutions, insertions, or deletions, from that of SEQ ID NO: 2. In some aspects, RAPGEF3 gene amplicons are obtained by using a reverse primer having 1, 2, 3, 4 or 5 nucleotide substitutions, insertions, or deletions, from that of SEQ ID NO: 2, and a Tₘ of within 80% to 120% that of the calculated (using NCBI BLAST method) Tₘ for SEQ ID NO: 2.

In some aspects, the methods for preparing gene amplicons or enriched nucleic acid samples further comprises the steps of end-repairing and dA-tailing before attaching adapters to the free nucleic acids. In some aspects, the steps of end-repairing and dA-tailing before attaching adapters to the free nucleic acids includes or excludes purifying the end-repaired products prior to the dA-tailing step, and includes or excludes purifying the dA-tailing products.

In one aspect, the free nucleic acids of the sample are not subjected to fragmentation. In one aspect, the free nucleic acids of the sample are subjected to fragmentation.

In one aspect, the sample from a subject is derived from saliva, blood, plasma, urine, cells, or tissue.

In some aspects, the method of preparing gene amplicons or enriched nucleic acid samples comprising target loci further comprises detecting the clonally amplified gene amplicons by PCR, multiplex PCR or sequencing.

In some aspects, this disclosure provides for a method of treating a subject having or suspected of having a vascular retinopathy, the method comprising:
a. obtaining or having obtained a sample from the subject;
b. detecting or having detected one or a plurality of gene variants of RAPGEF3: rs2240079, rs11168215, rs11168214 and rs61917617; ADCY7: rs55683248; ADCY9: rs2072341; PRKAR1A: rs8082254 and rs72847785; and ADCY4: rs3181252 and rs17256902 in the sample from the subject; and
c. administering a therapeutically effective amount of a modulator of RAPGEF3, ADCY7, or ADCY9 to the subject having one or a plurality of gene variants of RAPGEF3: rs2240079, rs11168215, rs11168214 and rs61917617; ADCY7: rs55683248; or ADCY9: rs2072341;
or administering a therapeutically effective amount of a modulator of PRKAR1A or ADCY4 to the subject having one or a plurality of gene variants of RAPGEF3: rs2240079, rs11168215, rs11168214 and rs61917617; ADCY7: rs55683248; or ADCY9: rs2072341;
or administering a therapeutically effective amount of a modulator of PRKAR1A or ADCY4 to a subject having one protective or no gene variant from the group consisting of: rs8082254, rs72847785, rs3181252 and rs17256902.

In some aspects, this disclosure provides for a method of administering a modulator to a subject, which is a therapeutic agent that alters the expression, level and/or activity of a gene or gene product (including RNA or protein products). In some aspects a modulator is an antagonist, e.g., a therapeutic agent that interferes with or inhibits the physiological action, e.g., expression, level and/or activity of a target, or that inhibits or interferes with the physiological action of a positive-regulator of the gene or gene product (e.g., in some aspects a positive-regulator increases the expression, level and/or activity of the target gene or gene product or decreases the expression, level and/or activity of a negative-regulator of the target that decreases the expression, level and/or activity of the target gene or gene product). In some aspects, the antagonist inhibits the gene or gene product directly, or indirectly by activating an inhibitor of the gene or gene product, or by inhibiting an activator of the gene to reduce the expression, level, and/or activity of the target gene or gene product.

In some aspects the modulator is an agonist, e.g., a therapeutic agent that activates or enhances the physiological action, e.g., expression, level and/or activity of a target gene or gene product, or that activates a positive regulator of the target (a gene or gene product that increases the expression, level and/or activity of a target or that interferes with or inhibits a negative regulator of the target). In some aspects, the agonist can activate the gene or gene product directly, or indirectly, e.g., by inhibiting an inhibitor of the gene or gene product, or by activating or upregulating an activator of the gene to increase the expression, level, and/or activity of the target gene or gene product.

In some aspects, a modulator is administered to a subject having a gene variant, to alter a gene or gene product to similar expression, level and/or activity as unaffected subjects who do not have the variant, or to provide similar expression, level and/or activity of a gene or gene product as subjects carrying protective gene variants against developing a vascular retinopathy. In some aspects, a modulator is administered to a subject to enhance the expression, level and/or activity of a gene or gene product in subjects. In some aspects, a modulator is administered to a subject to restore the expression, level and/or activity of a gene or gene product to baseline, for example to obtain the expression, level and/or activity of a non-carrier; and/or to provide a subject with no or a single protective allele with substantially the same expression, level and/or activity as in a subject carrying protective gene variants, e.g., to obtain the expression, level and/or activity of a subject carrying two protective alleles, e.g. rs8082254, rs72847785, rs3181252, and rs17256902. In some aspects, a modulator is administered to a subject to reduce the expression, level and/or activity of a gene or gene product in subjects.

In some aspects, this disclosure provides for a method of treating a subject, e.g., patient having ROP by administering a therapeutically effective amount of a modulator of RAPGEF3, ADCY7, ADCY9, PRKAR1A or ADCY4, the method comprising:
a. determining or having determined if the subject, e.g., patient has a mutation in RAPGEF3, ADCY7, ADCY9, PRKAR1A or ADCY4; and
b. if the subject, e.g., patient has a mutation in RAPGEF3, ADCY7, or ADCY9, further determining whether the patient has one or a plurality of gene variants of RAPGEF3: rs2240079, rs11168215, rs11168214 and rs61917617; ADCY7: rs55683248; ADCY9: rs2072341 then administering an a therapeutically effective amount of a modulator of RAPGEF3, ADCY7, ADCY9, PRKAR1A or ADCY4; or
c. if the subject, e.g., patient has a mutation in PRKAR1A or ADCY4, further determing whether the patient has one or a plurality of gene variants of PRKAR1A: rs8082254 and rs72847785 and ADCY4: rs3181252 and rs17256902 then administering a therapeutically effective amount of a modulator of PRKAR1A or ADCY4 if the subject, e.g., patient carries one or no protective gene variant.

In some aspects, this disclosure provides for a method of slowing vision loss or improving the vision in a subject having or suspected of having a vasulcar retinopathy, comprising administering to the subject a therapeutically effective amount of a modulator to RAPGEF3, ADCY7, ADCY9, PRKAR1A or ADCY4.

In some aspects, the modulator is an antagonist that is a gene knockdown agent, e.g., siRNA, shRNA, antisense RNA, or a gene knockout agent that is a transcription activator-like effector nuclease (TALEN) or a zinc finger nuclease (ZFN). In some aspects, the modulator is an antagonist, e.g. a negative allosteric modulator or competitive antagonist, that represses the gene or gene product's expression, level and/or activity.

In some aspects, the modulator is an agonist that is a gene activating agent, e.g., siRNA or shRNA targeting an upstream regulator of the target gene's expression, level and/or activity. In some aspects, the modulator is an agonist, e.g. a positive allosteric modulator, that enhances the gene or gene product's expression, level and/or activity.

In some aspects, the method of treating a subject having or suspected of having a vascular retinopathy, the method of treating a subject having ROP, or the method of slowing vision loss or improving the vision in a subject having or suspected of having a vascular retinopathy further comprises administering one or more additional active agents or supportive therapies for treating, preventing, or reducing the severity of an eye disorder to the subject. In some aspects, the one or more supportive therapies is selected from the group consisting of: surgery, laser therapy, photocoagulation, anti-angiogenic therapy, vitrectomy, scleral buckle surgery, and pneumatic retinopexy, or any combination thereof. In some aspects, the one or more additional active agents is selected from the group consisting of: VEGF antagonists and/or inhibitors, VEGF, placental growth factor (PIGF) inhibitor, bevacizumab, ranibizumab, aflibercept, Ca²⁺ inhibitors, flunarizine, nifedipine, cryotherapy, hyperbaric oxygenation, Na⁺ channel blockers, topiramate, iGluR antagonists, (MK-801, dextromethorphan, eliprodil, flupirtine, antioxidants, dimethylthiourea, alpha-lipoic acid, superoxide dismutase, catalase, desferrioxamine, mannitol, allopurinol, calcium dobesilate, trimetazidine, EGB-761, anti-inflammatory agents, cyclodiathermy, cyclocryotherapy, ocular filtering procedures, implantation of drainage valves, antiplatelet therapy, aspirin, ticlopidine, clopidogrel, anticoagulant therapy, warfarin, heparin, steroids, systemic or local corticosteroids, prednisone triamcinolone, fluocinolone acetonide, dexamethasonc, steroid-sparing immunosuppressants, cyclosporine, azathioprine, cyclophosphamide, mycophenolate, mofetil, infliximab, etanercept, dietary supplements, vitamin C, vitamin E, lutein, zinc, folic acid, vitamin B6, vitamin B12, zeaxanthin, a VEGF and PIGF inhibitor, or any combination thereof. In some aspects, the VEGF antagonists can include or exclude: Avastin (bevacizumab), Eylea (aflibercept), Lucentis (ranibizumab), Nexavar (sorafenib), Sprycel (dasatinib), Sutent (sunitinib), Tasigna (nilotinib), and Votrient (pazopanib).

In some aspects, the RAPGEF3, ADCY7, or ADCY9 modulator comprises a polypeptide capable of binding or sequestering RAPGEF3, ADCY7, or ADCY9. In some aspects, the polypeptide capable of binding or sequestering RAPGEF3, ADCY7, or ADCY9 comprises an antibody, antibody fragment, ScFv, Fv, Fd, Fab, Fab′, F(ab)′2, VH domain, VL domain, monoclonal antibody, polyclonal antibody, Fc or fusion protein or any combination thereof to said RAPGEF3, ADCY7, or ADCY9 or epitopic portion thereof.

In some aspects, the ADCY4 or PRKAR1A modulator comprises a polypeptide capable of binding or sequestering ADCY4 or PRKAR1A. In some aspects, the polypeptide capable of binding or sequestering ADCY4 or PRKAR1A comprises an antibody, antibody fragment, ScFv, Fv, Fd, Fab, Fab′, F(ab)′2, VH domain, VL domain, monoclonal antibody, polyclonal antibody, Fc or fusion protein or any combination thereof to said ADCY4 or PRKAR1A or epitopic portion thereof.

In one aspect, this disclosure provides for a method of screening for a gene knockout or gene knockdown treatment of retinopathy, the method comprising performing PCR for gene variants RAPGEF3: rs2240079, rs11168215, rs11168214 and rs61917617; ADCY7: rs55683248; and ADCY9: rs2072341 in a mammalian animal model of oxygen-induced retinopathy then administering the gene knockdown or gene knockout treatment of retinopathy to the animal model, then observing the knockdown or knockout effect.

In another aspect, this disclosure provides for a method of screening for a gene knock-in or gene activation treatment of a retinopathy, the method comprising performing PCR for the following gene variants RAPGEF3: rs2240079, rs11168215, rs11168214 and rs61917617; ADCY7: rs55683248; and ADCY9: rs2072341 in an animal model of oxygen-induced retinopathy, then administering the gene knock-in or gene activation treatment to of a retinopathythe animal model, then observing the knockdown or knockout effect.

In another aspect, this disclosure provides for a method of screening for a gene knockout or gene knockdown treatment of a retinopathy, the method comprising performing PCR for gene variants PRKAR1A: rs8082254 and rs72847785, and ADCY4: rs3181252 and rs17256902 in a mammalian animal model of oxygen-induced retinopathy, then administering the gene knockout or gene knockdown treatment of a retinopathy to the animal model, then observing the knock-in or gene activation effect.

In another aspect, this disclosure provides for a method of screening for a gene knock-in or gene activation treatment of a retinopathy, the method comprising performing PCR for gene variants PRKAR1A: rs8082254 and rs72847785, and ADCY4: rs3181252 and rs17256902 in a mammalian animal model of oxygen-induced retinopathy, then administering the gene knock-in or gene activation treatment of a retinopathy to the animal model, then observing the knock-in or gene activation effect.

It is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Allelic association analysis between ROP and no ROP participants. Manhattan plot showing the association test results of 534 SNVs and 13 indels with the most significant signals indicated above the dashed line p<0.05. Four points for chromosome 12 corresponding to RAPGEF3 SNVs, two points for chromosome 14 corresponding to ADCY4 SNVs, one point in chromosome 16 corresponding to an ADCY9 SNV, one point in chromosome 17 corresponding to an ADCY7 SNV, and two points in chromosome corresponding to PRKAR1A SNVs appear above the p<0.05 significance threshold. P values were obtained by Fisher's exact test.

FIGS. 2A-2C. Validation and combined analyses for RAPGEF3 variants rs61917617, rs11168215, and rs11168214. FIG. 2A Representative restriction digests with SacII, BsaWI and Bsu36I of a 661 bp RAPGEF3 amplicon from participants 1 to 5 of the validation cohort. Complete digestions of the 661 bp amplicon corresponded to a homozygote carrier participant 1 (Hom); partial digestions associated with a heterozygote carrier participant 4 (Het); and absence of digestion (uncut) was associated with non-carrier participants 2, 3 and 5 of the variants (−). u: undigested amplicon, −: no DNA; L: DNA ladder; fragment a: 373 bp; b: 288 bp; c: 401 bp; d: 260 bp; e: 504 bp; f: 157 bp. FIG. 2B RAPGEF3 variants rs61917617, rs11168215, and rs11168214 allele frequency ±SEM in ROP (n=12) versus no ROP (n=3) groups from the validation cohort. FIG. 2C Combined analysis for RAPGEF3 variants allele frequency ±SEM in ROP (n=30) versus no ROP (n=34) groups from the discovery and validation cohorts.

FIG. 3 is Table 1. Characteristics of participants. Number of participants: n; P values were obtained from unpaired Student's t test for birth weight and gestational age, and Chi squared test or Fisher's exact test for sex, race and ethnicity distribution.

FIG. 4 is Table 2. Genetic variants analysis for allelic association to ROP versus no ROP premature participants. Chr: Chromosome; Alt: alternative allele; No ROP: unaffected participants alternative allele frequency, (homozygote frequency), ROP: affected participants alternative allele frequency, (participant homozygote frequency); 95% CI: 95% confidence interval; Power: Confidence that the hypothesis that the variant is not associated with ROP is false at a level of 0.05; Population Frequency: Alternative allele frequency in the general population (Genomes 1000 project phase 3, release V3+; global population: www.ncbi.nlm.nih.gov/bioproject/PRJEB6930).

FIG. 5 is Table 3. Linkage disequilibrium analyses between relevant variants of the same gene measured by the squared correlation (r²).

FIG. 6 is Table 4. LC Sciences VariantBaits probes list for twenty components of the ADRβ signaling pathways. The components are: three ADRB subtypes (1, 2, and 3); ten ADCY isoforms, Class I to X including the non-cardiac ADCY VIII; four subtypes of protein kinase A (PRKAR1A, PRKAR2A, PRKAR1B, PRKAR2B); RAPGEF3, the tyrosine kinase Src, and cortactin.

### DETAILED DESCRIPTION

The inventions described and claimed herein have many attributes and embodiments including, but not limited to, those set forth or described or referenced in this Detailed Description. It is not intended to be all-inclusive and the inventions described and claimed herein are not limited to or by the features or embodiments identified in this Detailed Description, which is included for purposes of illustration only and not restriction.

### A. Certain Exemplary Definitions

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like. The terms “including”, “includes”, “having”, “has”, “with”, or variants thereof are used in either the detailed description and/or the claims, such terms are intended to be inclusive in a manner similar to the term “comprising”.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as values between 10 and 15. For example, it is also understood that each unit between two particular units are also disclosed. For example, if 10 and 15 are disclosed, then 11, 12, 13, and 14 are also disclosed. In this application, if a data point range is disclosed, it is understood that each unit from the lowest data point to the highest stated datapoint, including the first (lowest) and last (highest) data point is disclosed. For example, if a data point range 1-20 is disclosed, it is understood that data points 1, 2, 3, 4, 5, 6, 7, 8, 9, 10, 11, 12, 13, 14, 15, 16, 17, 18, 19, and 20, and each unit between any two particular units in the range are also disclosed. It is also understood that whenever a series of values are disclosed, that any range falling between any two of the recited values is also understood to be included.

In this specification and in the claims which follow, reference will be made to a number of terms which shall be defined to have the following meanings:

“Optional” or “optionally” means that the subsequently described event or circumstance may or may not occur, and that the description includes embodiments where said event or circumstance occurs and embodiments where it does not.

As used herein, the term “separation”, includes any means of substantially purifying one component from another (e.g., by filtration, magnetic attraction, etc.).

As used herein, the term “isolation” or “isolating”, includes any means of sorting one species of a type of genus from another species of a type of genus.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Animal models of oxygen-induced retinopathy (OIR) have been used to study the mechanisms underlying the pathogenesis of ROP (Madan, A. and Penn, J S. Animal models of oxygen-induced retinopathy. Front Biosci. 1; 8:d1030-43 (2003)). Due to their cost-effectiveness, high reproducibility, and consistent pattern of retinal neovascularization that is similar to that observed in humans, both murine and rodent OIR models have been popular models used to study the etiology of ROP (Heidary, G., Vanderveen, D., and Smith, L E. Retinopathy of Prematurity: Current Concepts in Molecular Pathogenesis. Semin Ophthalmol. 24(2): 77-81. (2009). Barnett, J M., et al. The development of the rat model of retinopathy of prematurity. Doc Ophthalmol. 120(1):3-12 (2010). Cringle, S J. and Yu, D-Y. Oxygen supply and consumption in the retina: implications for studies of retinopathy of prematurity. Doc Ophthalmol. 120(1):99-109 (2010)). Thus, the subject can be a human or veterinary patient. The term subject, in some embodiments, refers to a “patient” under the treatment of a clinician, e.g., physician.

As used herein, the terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder and those prone to have the condition or disorder or those in which the condition or disorder is to be prevented.

The term “preventing” means preventing in whole or in part, or ameliorating or controlling.

As used herein, the term “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of or reduces the intensity of one or more symptoms of the particular disease, condition, or disorder described herein.

As used herein, the term “substantially the same” means an amount of expression, level, and/or activity of a gene or gene product within 90% of baseline expression, level, and/or activity as in a subject unaffected by a disease, for example in a subject unaffected by a vascular retinopathy. “Substantially the same” can also mean an amount of expression, level, and/or activity of a gene or gene product within 80, 81, 82, 83, 84, 85, 86, 87, 88, 89, 90, 91, 92, 93, 94, 95, 96, 97, 98, 99, to 100% of the baseline expression, level, and/or activity as in a subject unaffected by unaffected by a disease, for example in a subject unaffected by a vascular retinopathy.

As described herein, any concentration range, percentage range, ratio range or integer range is to be understood to include the value of any integer within the recited range and, when appropriate, fractions thereof (including one-tenth and one-hundredth of an integer), unless otherwise indicated.

As used herein, the term “vascular retinopathy” includes, but is not limited to, retinopathy of prematurity (ROP), proliferative diabetic retinopathy (PDR), a retinal vein occlusion retinopathy (RVO), hypertensive retinopathy, branch retinal vein occlusion (BRVO), central retinal artery occlusion (CRAO), central retinal vein occlusion, chronic BRVO, Coats' Disease, cotton-wool spots, dot and blot hemorrhage, flame-shaped hemorrhage, hard exudates, hollenhorst plaques, inferior hemicentral retinal vein occlusion, juxtafoveal telangiectasia, optociliary shunt, preretinal hemorrhage, retinal artery macroaneurysm, sickle proliferative retinopathy, subretinal hemorrhage, superior hemicentral retinal vein occlusion, fibrovascular proliferation in PDR, neovascularization of the disc, nonproliferative diabetic retinopathy (NPDR), panretinal photocoagulation, retinal vessel occlusive disease, macular degeneration including age-related macular degeneration, or any neovascular retinal disease. In some embodiments, vascular retinopathy is neovascular retinopathy. In certain embodiments, the vascular retinopathy is ROP.

ROP is classified according to the locations (zones) and presence or absence of Type 1 ROP (Good, WV. Final result of the early treatment for retinopathy of immaturity (ETROP) randomized trial. Trans Am Opthahmol Soc. 102:233-250 (2004)). Type I ROP is defined as Zone I with plus disease (tortuosity and dilatation of retinal vessels) or stage 3 with or without plus disease, stage 2 and/or 3.

As used herein, the term “genetic factors” includes, but is not limited to, gene alterations and variants such as single nucleotide polymorphisms/single nucleotide variants (SNPs/SNVs), deletions, insertions, copy number variations, translocations, inversions, and structural variations. SNPs/SNVs include synonymous and nonsynonymous mutations. Nonsynonymous mutations include missense mutations, frame-shift mutations, nonsense mutations, and readthrough mutations. As used herein, the terms “risk allele”, “risk variant”, and “risk gene variant” include, but are not limited to a gene variant or SNP/SNV that is associated with risk to develop a disease, e.g. a retinopathy. As used herein, the terms “protective allele”, “protective variant”, and “protective gene variant” include, but are not limited to a gene variant or SNP/SNV that is associated with protection against developing a disease, e.g. a retinopathy. In some embodiments, a subject having a protective allele or protective variant is a subject that has one protective allele, e.g. rs8082254, rs72847785, rs3181252 and rs17256902.

Polymorphisms in the genes involved in ADRβ pathway have been investigated for protection against ROP (Good et al. β-Blocking and racial variation in the severity of retinopathy of prematurity. Arch Ophthalmol. 130(1):117-118 (2012)). Such polymorphisms are more common in African-Americans, and render the affected individual relatively insensitive to antagonists to ADRβ (beta-blockers) therapies for several conditions (Liggett et al. A GRK5 polymorphism that inhibits beta-adrenergic receptor signaling is protective in heart failure. Nat Med. 14(5):510-517 (2008)).

As used herein, the terms “polynucleotide”, “nucleic acid” and “nucleic acid molecule” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”).

As used herein, the term “all or a portion of” refers to the full nucleotide sequence of a gene or a portion of a gene as a polymeric form of nucleotides of any length thereof, including contiguous fragments of an indicated nucleotide acid sequence, and may comprise ribonucleotides, deoxyribonucleotides, analogs thereof, or mixtures thereof.

In some embodiments of any aspect herein, the methods disclosed herein comprise enrichment, e.g., amplification or hybridization enrichment, and library preparation in anticipation of downstream high throughput, e.g., massively parallel sequencing. An assay can include one or two PCR master mixes, one or two thermostable polymerases, one or two primer mixes and library adapters. In some embodiments, adapters are added to sample fragments, processed fragments and/or DNA derived from sample fragments or processed fragments, or copies thereof, by ligation, PCR, or by molecular inversion probes which can be circularized and amplified by rolling circle replication, or which can be circularized and linearized prior to amplification. In some embodiments, a sample of DNA may be enriched, e.g., using bait hybridization, or amplified for a number of cycles by using a first set of amplification primers that comprise target specific regions and non-target specific tag regions and a first PCR master mix. The tag region can be any sequence, such as a universal tag region, a capture tag region, an amplification tag region, a sequencing tag region, a unique molecular identifier (UMI) tag region, and the like. In some embodiments, a tag region can be the template for amplification primers utilized in a second or subsequent round of amplification, for example for library preparation. An aliquot of the first amplified sample can be removed and amplified a second time using a second set of amplification primers that are specific to the tag region, e.g., a universal tag region or an amplification tag region, of the first amplification primers which may comprise of one or more additional tag sequences, such as sequence tags specific for one or more downstream sequencing workflows, and the same or a second PCR master mix for sequencing.

In some embodiments, processing DNA fragments may include or exclude isolating or extracting DNA from a biological sample, blunt ending, end repairing, phosphorylation, dephosphorylation, and/or dA tailing. DNA “derived” from biological sample fragments may include or exclude any processing step and may further also include or exclude amplifying (exponentially or linearly, using target specific or universal primers), enzymatic cleavage, circularization, or ligation that takes place prior to the recited step that uses DNA derived from a source as input DNA.

Following library creation, the library can be optionally purified and quantitated. In some embodiments, purification can be performed by processing the sample through a substrate such as AMPURE XP Beads (Beckman Coulter) which serves to purify the DNA fragments away from reaction components. In some methods, the purification can be performed by incorporating a biotin into one of the primers of the second amplification primer set then the library fragments could be capturing using a streptavidin moiety on a bead for example. Utilizing the capture strategy, the libraries could also be normalized and quantitated using bead-based normalization. However, libraries can be purified and quantitated, or pooled and quantitated if multiple reactions are being performed, without the use of bead-based normalization. For example, libraries could also be quantitated by gel electrophoretic methods, microfluidics-based automated electrophoresis methods, e.g., using the Agilent Bioanalyzer, qPCR, spectrophotometric methods, quantitation kits (e.g., PicoGreen™, Nanodrop, etc.) and the like as known in the art. Following quantitation, the library can then be sequenced by massively parallel sequencing.

The size of the nucleic acid molecules may vary greatly using the methods and compositions disclosed herein. It would be appreciated by those skilled in the art that the nucleic acid molecules amplified from a target sequence comprising a tandem repeat (e.g., STR) may have a large size, while the nucleic acid molecules amplified from a target sequence comprising a SNP may have a small size. In some embodiments, the nucleic acid molecules may comprise from less than a hundred nucleotides to hundreds or even thousands of nucleotides. In some embodiments, the size of the nucleic acid molecules may have a range that is between 3-bp to 1 kb. The size of the nucleic acid molecules may have a range that is between any two values of about 50 bp, about 60 bp, about 70 bp, about 80 bp, about 90 bp, about 100 bp, about 110 bp, about 120 bp, about 130 bp, about 140 bp, about 150 bp, about 200 bp, about 300 bp, about 400 bp, about 500 bp, about 600 bp, about 700 bp, about 800 bp, about 900 bp, about 1 kb, or more. In some embodiments, the minimal size of the nucleic acid molecules may be a length that is, is about, or is less than, 50 bp, 60 bp, 70 bp, 80 bp, 90 bp, or 100 bp. In some embodiments, the maximum size of the nucleic acid molecules may be a length that is, is about, or is more than, 100 bp, 150 bp, 200 bp, 250 bp, 300 bp, 350 bp, 400 bp, 450 bp, 500 bp, or 1 kb.

For cluster generation, the library fragments are immobilized on a substrate which comprises homologous oligonucleotide sequences for capturing and immobilizing the DNA library fragments. The immobilized DNA library fragments can be amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to as “clustered arrays”. The products of solid-phase amplification reactions such as those described in U.S. Pat. Nos. 7,985,565 and 7,115,400 are referred to as “bridged” structures formed by annealing of pairs of immobilized polynucleotide strands and immobilized complementary strands, both strands being immobilized on the solid support at the 5′ end, preferably via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized. The methods described herein are not limited to any particular sequencing preparation methodology or sequencing platform and can be amenable to other sequencing platform preparation and sequencing methods.

In some embodiments, methods described herein involve unique molecular identifiers (UMI). A UMI comprises a randomly selected stretch of nucleotides that can be used during sequencing to correct for PCR and sequencing errors, thereby adding an additional layer of error correction to sequencing results. UMIs could be from, for example, 3-20 nucleotides long (e.g., 3, 4, 5, 6, 7, 8, 9, 10, 11, 12, 13, 14, 15, 16, 17. 18, 19, or 20, or any range between any of those lengths), however the number will depend on the amount of input DNA. For example, if 1 ng DNA is used to target around 250 sites, then it is anticipated that approximately 350 copies×250 targets would be needed, so approximately 90,000 different UMIs. If more DNA is utilized, for example 10 ng, then approximately 1 million different UMIs could be needed. All PCR duplicates from the same PCR reaction would have the same UMI sequence, as such the duplicates can be compared and any errors in the sequence such as single base substitutions, deletions, insertions (i.e., stutter in PCR) can be excluded from the sequencing results bioinformatically.

In some embodiments, a primer of the present methods can comprise one or more tag sequences. The tag sequences can be one or more of primer sequences that are not homologous to the target sequence, but for example can be used as templates for one or more amplification reactions. The tag sequence can be a capture sequence, for example a hapten sequence such as biotin that can be used to purify amplicons away from reaction components. The tag sequences can be sequences such as adapter sequences that are advantageous for capturing the library amplicons on a substrate for example for bridge amplification in anticipation of sequence by synthesis technologies as described herein. Further, tag sequences can be UMI tags of typically between, for example, 3-20 nucleotides comprised of a randomized stretch of nucleotides that can be used for error correction during library preparation and/or sequencing methods.

### B. Certain Exemplary Methods

Human clinical studies of the use of beta-blockers to prevent ROP have demonstrated mixed results in addition to safety concerns (Ristori et al. Role of the adrenergic system in a mouse model of oxygen-induced retinopathy: antiangiogenic effects of beta-adrenoreceptor blockade. Invest Ophthalmol Vis Sci. 52(1):155-170 (2011). Chen et al. Propranolol inhibition of β-adrenergic receptor does not suppress pathologic neovascularization in oxygen-induced retinopathy. Invest Ophthalmol Vis Sci. 53(6):2968-2977 (2012). Kaempfen et al. Beta-blockers for prevention and treatment of retinopathy of prematurity in preterm infants. Cochrane Database Syst Rev. 3(3):CD011893 (2018)). ADRβ are a subfamily of guanosine triphosphate-binding protein (G-protein) coupled receptors (GPCRs) including β1, β2 and β3 that are expressed in the retina (Cammalleri, M., et al. The β3 adrenoceptor in proliferative retinopathies: “Cinderella” steps out of its family shadow. Pharmacol Res. 190:106713 (2023)). Activation of stimulatory G protein (Gs) following stimulation of ADRβ or other receptors leads to the modulation of ADCY activity to increase levels of 3′,5′-cyclic adenosine monophosphate (cAMP) which in turn, activates exchange proteins activated by cAMP (EPAC1 and 2) encoded by RAPGEF3 and 4, as well as PRKA family members (Bártori, R., et al. Differential mechanisms of adenosine- and ATPγS-induced microvascular endothelial barrier strengthening. J Cell Physiol. 234(5):5863-5879 (2019). Formoso, K., et al. Role of EPACl Signalosomes in Cell Fate: Friends or Foes?Cells. 9(9):1954 (2020). Cammalleri, M., et al. The β3 adrenoceptor in proliferative retinopathies: “Cinderella” steps out of its family shadow. Pharmacol Res. 190:106713 (2023). Erofeeva, N., et al. Multiple Roles of cAMP in Vertebrate Retina. Cells. 12,1157 (2023)). There is also evidence that stimulation of ADRβ can inactivate ADCY when coupled to inhibitory G protein (Gi) (de Lucia, C., et al. New Insights in Cardiac β-Adrenergic Signaling During Heart Failure and Aging. Front Pharmacol. 9:904 (2018). Formoso, K., et al. Role of EPAC1 Signalosomes in Cell Fate: Friends or Foes? Cells. 9(9):1954 (2020)). EPAC1, but not EPAC2, has been implicated in regulating junctional complexes in both the retinal and endocardial endothelial barriers (Parnell, E. and Yarwood, SJ. (2014). Liu, L., et al. Epac1 protects the retina against ischemia/reperfusion-induced neuronal and vascular damage. PLoS One. 13(9): e0204346 (2018). Garcia-Ponce, A., et al. Epac1 Is Crucial for Maintenance of Endothelial Barrier Function through A Mechanism Partly Independent of Rac1. Cells. 9(10):2170 (2020)). The Retinal endothelial barriers are known to be affected during ROP and thus it is plausible that dysfunction of ADRβ pathway could contribute to events that promote ROP development (Bharadwaj, A S., et al. Role of the retinal vascular endothelial cell in ocular disease. Prog Retin Eye Res. 32:102-180 (2013)). As described further herein, in light of the controversy of the role of the ADRβ pathway in ROP, an observational case-control study on human premature infants with severe ROP (Type 1) and without any ROP was conducted to assess if genetic variants in genes of the ADRβ pathway were associated with ROP.

In some embodiments, this disclosure provides for methods of detecting genetic factors, e.g., gene variants and mutations not previously detected in subjects with vascular retinopathies which can include or exclude ROP.

In some embodiments, the genetic factors are detected in genes of the ADRβ pathway. In certain embodiments, the genetic factors detected in the ADRβ pathway include, but are not limited to, one or more gene variants in RAPGEF3, ADCY4, ADCY7, ADCY9, and PRKAR1A. In certain embodiments, the gene variants detected in the ADRβ pathway include or exclude one or more of rs8082254, rs72847785, rs3181254, rs17256902, rs2240079, rs61917617, rs11168215, rs11168214, rs55683248, rs2072341.

The present disclosure also relates to methods for detecting gene variants in a sample from a subject having or suspected of having a vascular retinopathy. In some embodiments, the method for detecting gene variants comprises obtaining or having obtained a sample comprising free nucleic acids from a subject, attaching adapters to the free nucleic acids for form adapter-nucleic acids, optionally amplifying the adapter-nucleic acids to generate amplicons, and detecting whether gene variants are present in the amplicons. In certain embodiments, detecting whether gene variants or target loci are present in the amplicons comprises the steps: (i) contacting the amplicons with a flow cell or solid surface comprising a plurality of nucleotides connected thereto to generate immobilized amplicons, wherein the oligonucleotides bind to the adapter, (ii), clonally amplifying the immobilized amplicons to generate clonally amplified amplicons, and (iii) sequencing all of or at least a portion of the sequences of the clonally amplified gene amplicons. In other embodiments, the method of detecting gene variants comprises additional step of sequencing at least a portion of the amplicons by sequencing. In some embodiments, sequencing is targeted sequencing or random. In some embodiments, the sequencing is performed by massively parallel sequencing.

The present disclosure also relates to methods for preparing gene amplicons or enriched nucleic acid samples from a subject having or suspected of having a vascular retinopathy. In some embodiments, the method comprises obtaining or having obtained a sample comprising free nucleic acids from a subject, attaching adapters to the free nucleic acids for form adapter-nucleic acids, and amplifying the adapter-nucleic acids to generate amplicons. In certain embodiments, the methods for preparing gene amplicons or enriched nucleic acid samples further comprises the steps: (i) contacting the amplicons with a flow cell or solid surface comprising a plurality of nucleotides connected thereto to generate immobilized amplicons, wherein the oligonucleotides bind to the adapter, (ii), clonally amplifying the immobilized amplicons to generate clonally amplified amplicons, and (iii) sequencing all of or at least a portion of the sequences of the clonally amplified gene amplicons.

In other embodiments, the methods for preparing gene amplicons or enriched nucleic acid samples further comprises the steps of end-repairing and dA-tailing before attaching adapters to the free nucleic acids. In certain embodiments, the steps of end-repairing and dA-tailing before attaching adapters to the free nucleic acids includes or excludes purifying the end-repaired products prior to the dA-tailing step, and includes or excludes purifying the dA-tailing products prior to attaching the adapters.

In some embodiments, the free nucleic acids of the sample are not subjected to fragmentation.

### Obtaining the Sample

In some embodiments, the sample from a subject is obtained or derived from the subject's saliva, blood, plasma, urine, cells, or tissue. In certain embodiments, the sample is plasma or serum. Plasma or serum makes up roughly 55% of whole blood. In certain embodiments, the sample contains an amount of nucleic acids. In some embodiments, obtaining or having obtained the sample results in disrupting or lysing cells in the sample to derive free nucleic acids. Thus, in some embodiments, the subject's sample comprises cellular nucleic acids. In some embodiments, cellular nucleic acids make up less than about 1% of the total cellular nucleic acids in the sample. In some embodiments, cellular nucleic acids make up less than about 5% of the total cellular nucleic acids in the sample. In some embodiments, cellular nucleic acids make up less than about 10% of the total cellular nucleic acids in the sample. In some embodiments, cellular nucleic acids make up less than about 20% of the total cellular nucleic acids in the sample. In some embodiments, cellular nucleic acids make up more than about 50% of the total cellular nucleic acids in the sample. In some embodiments, cellular nucleic acids make up less than about 90% of the total cellular nucleic acids in the sample.

In some embodiments, this disclosure provides for methods for obtaining or having obtained a sample for the methods as described herein. A sample may be obtained directly (e.g., a doctor takes a blood sample from a subject). A sample may be obtained indirectly (e.g., through shipping, by a technician from a doctor or a subject). In some embodiments, the sample is a biological fluid such as saliva, blood, plasma, urine, cerebrospinal fluid, serum, vitreous, sputum, tears, perspiration, saliva, mucosal excretions, mucus, amniotic fluid, lymph fluid, or cells. In some embodiments, the sample is a swab sample (e.g., buccal swab, vaginal and/or cervical swab). In some embodiments, this disclosure provides for methods comprising obtaining or having obtained whole blood, plasma, serum, urine, saliva, cerebrospinal fluid, interstitial fluid, or vaginal fluid. In some embodiments, this disclosure provides for methods comprising obtaining or having obtained a blood sample via a finger prick. In some embodiments, this disclosure provides for methods comprising obtaining or having obtained a blood sample via a single finger prick. In some embodiments, this disclosure provides for methods comprising obtaining or having obtained a blood sample with not more than a single finger prick. In some embodiments, the blood sample is obtained via a finger prick only after the initial perfusion of blood is discarded (e.g., finger is pricked, initial blood sample is wiped clean, and second blood sample is collected). In some embodiments, this disclosure provides for methods comprising obtaining or having obtained capillary blood (e.g., blood obtained from a finger or a prick of the skin). In some embodiments, methods comprise squeezing or milking blood from a prick to obtain a desired volume of blood. In other embodiments, methods do not comprise squeezing or milking blood from a prick to obtain a desired volume of blood. While a finger prick is a common method for obtaining or having obtained capillary blood, other locations on the body would also be suitable, e.g., toe, heel, arm, palm, shoulder, earlobe. In some embodiments, this disclosure provides for methods comprising obtaining or having obtained a blood sample without a phlebotomy. In some embodiments, this disclosure provides for methods comprising obtaining or having obtained capillary blood. In some embodiments, this disclosure provides for methods comprising obtaining or having obtained venous blood. In some embodiments, methods disclosed herein do not comprise obtaining or having obtained venous blood (e.g., blood obtained from a vein). In some embodiments, methods comprise obtaining or having obtained tissue sample. In some embodiments, methods comprise obtaining or having obtained a sample, such as a tissue sample, via a biopsy. In some embodiments, methods comprise obtaining or having obtained a biological fluid via a liquid biopsy. In some embodiments, methods comprise obtaining or having obtained a biological fluid via aspiration, syringe, pipetting, or direct collection into a collecting vessel (e.g., heel stick).

In some embodiments, this disclosure provides for methods comprising obtaining or having obtained samples with fragmented nucleic acids. The sample may have been subjected to conditions that are not conducive to preserving the integrity of nucleic acids. In some embodiments, the sample may be a plasma sample. Plasma samples are often fragmented. The sample may have been frozen and thawed. The sample may have been exposed to air, heat, light, or chemicals or enzymes that degrade nucleic acids. In some embodiments, methods comprise obtaining or having obtained a tissue sample wherein the tissue sample comprises fragmented nucleic acids. In some embodiments, methods comprise obtaining or having obtained a tissue sample wherein the tissue sample comprises nucleic acids and fragmenting the nucleic acids to produced fragmented nucleic acids. In some embodiments, the tissue sample is a frozen sample. In some embodiments, the sample is a preserved sample. In some embodiments the tissue sample is a fixed sample (e.g. formaldehyde-fixed). Methods may comprise isolating the (fragmented) nucleic acids from the sample.

In some embodiments, this disclosure provides for methods comprising obtaining or having obtained a sample from a subject, wherein the sample contains at least one nucleic acid of interest. In some embodiments, the nucleic acid of interest may be DNA from the saliva, blood, plasma, urine, cells, or tissue (e.g., eye) of a subject. In some embodiments, this disclosure provides for methods comprising obtaining or having obtained a sample from the subject, wherein the sample contains about 1 to about 100 nucleic acids. In some embodiments, the at least one nucleic acid is represented by a sequence that is unique to a target gene disclosed herein.

Isolating and Purifying Nucleic Acids from the Sample

In some embodiments of any aspect herein, this disclosure provides for methods comprising isolating or having isolated nucleic acids from a biological sample. In some embodiments, this disclosure provides for methods comprising purifying or having purified nucleic acids from a biological sample. As used herein the term “biological sample” means a biological fluid (e.g., saliva, blood, plasma, urine, cerebrospinal fluid, serum, vitreous, sputum, tears, perspiration, saliva, mucosal excretions, mucus, amniotic fluid, lymph fluid), cells, or a tissue sample obtained from a subject. In some embodiments, this disclosure provides for methods comprising isolating or having isolated and purifying or having purified nucleic acids from a biological sample. Isolation of cell-free nucleic acids may be accomplished using any means, including, but not limited to, the use of commercial kits and protocols provided by companies such as Life Technologies, Promega, Affymetrix, Sigma Aldrich, or IBI. In some embodiments, this disclosure provides for methods comprising removing or having removed non-nucleic acid components from a biological sample described herein. The nucleic acids derived therefrom can include any or all the following manipulations described herein, including any method of isolating, purifying, enriching, and one or more steps involved in preparing a library of nucleic acids for detection from the nucleic acids.

In some embodiments, isolating or purifying comprises removing unwanted non-nucleic acid components from a biological sample. In some embodiments, isolating or having isolated and purifying or having purified nucleic acids from a biological sample comprises removing at least 5%, at least 10%, at least 20%, at least 30%, at least 40%, at least 50%, at least 60%, at least 70%, at least 80%, or at least 90% of unwanted non-nucleic acid components from a biological sample. In some embodiments, isolating or having isolated and purifying or having purified nucleic acids from a biological sample comprises removing at least 95% of unwanted non-nucleic acid components from a biological sample to obtain a sample derived from the initial sample. In some embodiments, isolating or having isolated and purifying or having purified nucleic acids from a biological sample comprises removing at least 97% of unwanted non-nucleic acid components from a biological sample. In some embodiments, isolating or having isolated and purifying or having purified nucleic acids from a biological sample comprises removing at least 98% of unwanted non-nucleic acid components from a biological sample. In some embodiments, isolating or having isolated and purifying or having purified nucleic acids from a biological sample comprises removing at least 99% of unwanted non-nucleic acid components from a biological sample. In some embodiments, isolating or having isolated and purifying or having purified nucleic acids from a biological sample comprises removing at least 95% of unwanted non-nucleic acid components from a biological sample. In some embodiments, isolating or having isolated and purifying or having purified nucleic acids from a biological sample comprises removing at least 97% of unwanted non-nucleic acid components from a biological sample. In some embodiments, isolating or having isolated and purifying or having purified nucleic acids from a biological sample comprises removing at least 98% of unwanted non-nucleic acid components from a biological sample. In some embodiments, isolating or having isolated and purifying or having purified nucleic acids from a biological sample comprises removing at least 99% of unwanted non-nucleic acid components from a biological sample.

In some embodiments, this disclosure provides for methods comprising isolating or having isolated and purifying or having purified DNA, RNA, or a subset of desired nucleic acids from a mix of nucleic acids derived from a biological sample. In some embodiments, a subset of desired nucleic acids can include, but are not limited to amplicons, cDNA, dsDNA, ssDNA, plasmid DNA, high molecular weight DNA, chromosomal DNA, genomic DNA (gDNA), mitochondrial DNA (mtDNA), mRNA, rRNA, tRNA, and nRNA, dsRNA. In some embodiments, methods may comprise analyzing only DNA. In methods wherein only DNA is analyzed, RNA is unwanted and creates undesirable background noise or contamination to the DNA; therefore, in some embodiments, the removed nucleic acid components are discarded. In some embodiments, this disclosure provides for methods comprising removing RNA from a biological sample. In some embodiments, this disclosure provides for methods comprising removing mRNA from a biological sample. In some embodiments, this disclosure provides for methods comprising removing microRNA from a biological sample. In some embodiments, removing nucleic acid components comprises contacting the nucleic acid components with an oligonucleotide capable of hybridizing to the nucleic acid, wherein the oligonucleotide is conjugated, attached or bound to a capturing device (e.g., bead, column, matrix, nanoparticle, magnetic particle, etc.). In some embodiments, the removed nucleic acid components are discarded.

In some embodiments, this disclosure provides for methods comprising isolating or having isolated and purifying or having purified nucleic acids from one or more non-nucleic acid components of a biological sample. Non-nucleic acid components may also be considered unwanted substances. Non-limiting examples of non-nucleic acid components include cells (e.g., blood cells), cell fragments, extracellular vesicles, lipids, proteins or a combination thereof. Additional non-nucleic acid components are described herein and throughout. It should be noted that while methods may comprise isolating and/or purifying nucleic acids, they may also comprise analyzing a non-nucleic acid component of a sample that is considered an unwanted substance in a nucleic acid purifying step. Isolating or having isolated and purifying or having purified may comprise removing components of a biological sample that would inhibit, interfere with or otherwise be detrimental to the later process steps such as nucleic acid amplification or detection.

In some embodiments, purifying or having purified nucleic acids does not comprise washing the nucleic acids with a wash buffer. In some embodiments, purifying or having purified nucleic acids comprises capturing the nucleic acids with a nucleic acid capturing moiety to produce captured nucleic acids. Non-limiting examples of nucleic acid capturing moieties are silica particles and paramagnetic particles. In some embodiments, purifying or having purified nucleic acids comprises passing the sample comprising the captured nucleic acids through a hydrophobic phase (e.g., a liquid or wax). The hydrophobic phase retains impurities in the sample that would otherwise inhibit further manipulation (e.g., amplification, sequencing) of the nucleic acids.

Isolating and/or purifying may occur with the use of a sample purifier. In some embodiments, isolating or having isolated and purifying or having purified nucleic acids comprises removing non-nucleic acid components from a biological sample described herein. In some embodiments, isolating or having isolated and purifying or having purified nucleic acids comprises discarding non-nucleic acid components from a biological sample. In some embodiments, isolating or having isolated and purifying or having purified nucleic acids comprises collecting, processing and analyzing the non-nucleic acid components. In some embodiments, the non-nucleic acid components may be considered biomarkers because they provide additional information about the subject.

In some embodiments, isolating or having isolated and purifying or having purified nucleic acids comprise lysing a cell. In some embodiments, isolating or having isolated and purifying or having purified nucleic acids avoids lysing a cell. In some embodiments, isolating or having isolated and purifying or having purified nucleic acids does not comprise lysing a cell. In some embodiments, isolating or having isolated and purifying or having purified nucleic acids does not comprise an active step intended to lyse a cell. In some embodiments, isolating or having isolated and purifying or having purified nucleic acids does not comprise intentionally lysing a cell. Intentionally lysing a cell may include mechanically disrupting a cell membrane (e.g., shearing). Intentionally lysing a cell may include contacting the cell with a lysis reagent.

In some embodiments, isolating or having isolated and purifying or having purified nucleic acids comprises separating components of a biological sample disclosed herein. In some embodiments, isolating or having isolated and purifying or having purified nucleic acids may comprise separating plasma from blood. In some embodiments, isolating or having isolated and purifying or having purified nucleic acids comprises centrifuging the biological sample, filtering the biological sample, contacting the sample with a solid phase support, or using solid phase extraction, in order to separate components of a biological sample, or a combination thereof. Obtaining plasma may comprise allowing blood to be subjected to gravity (e.g., sedimentation). In some embodiments, methods comprise subjecting the blood to vertical filtration. Obtaining plasma may comprise subjecting blood to a material that wicks a portion of the blood away from non-nucleic acid components of the blood. In some embodiments, methods comprise subjecting the blood to a sample purifier comprising a filter matrix for receiving whole blood, the filter matrix having a pore size that is prohibitive for cells to pass through, while plasma can pass through the filter matrix uninhibited. Such vertical filtration and filter matrices are described for devices disclosed herein.

In some embodiments, isolating or having isolated and purifying or having purified nucleic acids comprises filtering the biological sample in order to remove non-nucleic acid components from the biological sample. In some embodiments, isolating or having isolated and purifying or having purified nucleic acids comprises filtering the biological sample in order to capture nucleic acids from the biological sample. In some embodiments, removing non-nucleic acid components may comprise centrifuging the biological sample. In some embodiments, removing non-nucleic acid components may comprise contacting the biological sample with a binding moiety.

Isolating or having isolated and purifying or having purified may comprise capturing an extracellular vesicle or extracellular microparticle in the biological sample with a binding moiety. In some embodiments, the extracellular vesicle contains at least one of DNA and RNA.

### Enrichment of Nucleic Acids

In some embodiments, this disclosure provides for methods of enriching nucleic acids isolated and purified from a biological sample, e.g. by amplification or hybridizing enrichment such as bait capture to provide a sample enriched in selected or target nucleic acids. In some embodiments, methods comprise performing targeted sequencing on the enriched nucleic acids, which may comprise amplicons of nucleic acids in the biological sample, obtained by whole genome or targeted amplification.

In some embodiments, enriching nucleic acid components comprises separating the nucleic acid components on a gel by size. In some embodiments, this disclosure provides for methods comprising removing DNA from the biological sample. In some embodiments, this disclosure provides for methods comprising capturing DNA from the biological sample. In some embodiments, this disclosure provides for methods comprising selecting DNA from the biological sample. In some embodiments, the cell-free DNA has a minimum length. In some embodiments, the minimum length is about 50 base pairs. In some embodiments, the minimum length is about 100 base pairs. In some embodiments, the minimum length is about 110 base pairs. In some embodiments, the minimum length is about 120 base pairs. In some embodiments, the minimum length is about 140 base pairs. In some embodiments, the DNA has a maximum length. In some embodiments, the maximum length is about 180 base pairs. In some embodiments, the maximum length is about 200 base pairs. In some embodiments, the maximum length is about 220 base pairs. In some embodiments, the maximum length is about 240 base pairs. In some embodiments, the maximum length is about 300 base pairs. Size based separation would be useful for other categories of nucleic acids having limited size ranges (e.g., microRNAs).

In some embodiments, enriching methods comprise capturing a nucleosome in a biological sample and analyzing nucleic acids attached to the nucleosome. In some embodiments, methods comprise capturing an exosome in a biological sample and analyzing nucleic acids attached to the exosome. Capturing nucleosomes and/or exosomes may preclude the need for a lysis step or reagent, thereby simplifying the method and reducing time from sample collection to detection. In some embodiments, enriching nucleic acids comprises lysing and performing sequence specific capture of a target nucleic acid with “bait” in a solution followed by binding of the “bait” to solid supports such as magnetic beads. In some embodiments, methods comprise performing sequence specific capture in the presence of a recombinase or helicase to reduce the need for heat denaturation of a nucleic acid thereby speeding up the detection step.

In some embodiments, enriching nucleic acids comprises subjecting a biological sample, or a fraction thereof, or a modified version thereof, to a binding moiety. The binding moiety may be capable of binding to a component of a biological sample and removing it to produce a modified sample depleted of cells, cell fragments, nucleic acids or proteins that are unwanted or of no interest. In some embodiments, enriching purified nucleic acids comprises subjecting a biological sample to a binding moiety to reduce unwanted substances or non-nucleic acid components in a biological sample. In some embodiments, enriching purified nucleic acids comprises subjecting a biological sample to a binding moiety to produce a modified sample enriched with target cell, target cell fragments, target nucleic acids or target proteins. The resulting cell-bound binding moieties can be captured and enriched for with antibodies or other methods, e.g., low speed centrifugation.

In some embodiments, this disclosure provides for methods comprising amplifying at least one nucleic acid in a sample to produce at least one amplification product. The at least one nucleic acid may be a cell-free nucleic acid or nucleic acid obtained from a cell in a sample. The sample may be a biological sample disclosed herein or a fraction or portion thereof. In some embodiments, methods comprise producing a copy of the nucleic acid in the sample and amplifying the copy to produce the at least one amplification product. In some embodiments, methods comprise producing a reverse transcript of the nucleic acid in the sample and amplifying the reverse transcript to produce the at least one amplification product.

In some embodiments, methods comprise performing whole genome amplification. In some embodiments, methods do not comprise performing whole genome amplification. The term, “whole genome amplification” also referred to as “random amplification” may refer to amplifying all of the cell-free nucleic acids in a biological sample. The term, “whole genome amplification” may refer to amplifying at least 90% of the cell-free nucleic acids in a biological sample. Whole genome may refer to multiple genomes. Whole genome amplification may comprise amplifying cell-free nucleic acids from a biological sample of a subject having an infection, wherein the biological sample comprises cell-free nucleic acids from the subject and a pathogen.

In some embodiments, targeted sequencing is performed using bait-capture methods, wherein primers functionalized with a biotin can be targeted to selected regions of the genome, and the extended primers can be isolated (optionally for further isolation and/or amplification) by streptativin beads. In some embodiments, targeted sequencing can be performed using primers targeted to selected regions of the genome, wherein said regions are preferentially enriched and/or amplified relative to other regions in the genome.

In some embodiments, this disclosure provides for methods comprising amplifying a nucleic acid, wherein amplifying comprises performing an isothermal amplification of the nucleic acid. Non-limiting examples of isothermal amplification are as follows: loop-mediated isothermal amplification (LAMP), strand displacement amplification (SDA), helicase dependent amplification (HDA), nicking enzyme amplification reaction (NEAR), and recombinase polymerase amplification (RPA). In some embodiments, the isothermal amplification is high throughput involving parallel sample processing. In some embodiments, the high throughput isothermal amplification involves amplifying a nucleic acid in 12, 24, 36, 48, 60, 72, 84, 96, 108, or more samples in parallel. In some embodiments, the high throughput isothermal amplification involves amplifying a nucleic acid in between 12-24, 24-36, 36-48, 48-60, 70-72, 72-84, 84-96, 96-108, 108-120, 120-132, 132-144, 144-156-156-168, 168-180, 180-192, 192-204, 204-216, 216-228, 228-240, 240-252, or 252-264, samples in parallel. In some embodiments, the high throughput isothermal amplification involves amplifying a nucleic acid in at least 90, 100, 200, 300, 400, 500, 600, 700, 800, 900, 1,000, 1,100, 1,200, 1,300, 1,400, or 1,500 samples in parallel.

Amplification methods can include isothermal amplification. In some embodiments, amplification is isothermal with the exception of an initial heating step before isothermal amplification begins. A number of isothermal amplification methods, each having different considerations and providing different advantages, can be used (Zanoli and Spoto, 2013, “Isothermal Amplification Methods for the Detection of Nucleic Acids in Microfluidic Devices,” Biosensors 3: 18-43; Fakruddin, et al., 2013, “Alternative Methods of Polymerase Chain Reaction (PCR),” Journal of Pharmacy and Bioallied Sciences 5(4): 245-252). In some embodiments, any appropriate isothermic amplification method is used. In some embodiments, the isothermic amplification method used is selected from: Loop Mediated Isothermal Amplification (LAMP); Nucleic Acid Sequence Based Amplification (NASBA); Multiple Displacement Amplification (MDA); Rolling Circle Amplification (RCA); Helicase Dependent Amplification (HDA); Strand Displacement Amplification (SDA); Nicking Enzyme Amplification Reaction (NEAR); Ramification Amplification Method (RAM); and Recombinase Polymerase Amplification (RPA).

In some embodiments, the amplification method is Nucleic Acid Sequence Based Amplification (NASBA). NASBA (also known as 3SR, and transcription-mediated amplification) is an isothermal transcription-based RNA amplification system. Three enzymes (avian myeloblastosis virus reverse transcriptase, RNase Hand T7 DNA dependent RNA polymerase) are used to generate single-stranded RNA. In certain cases, NASBA can be used to amplify DNA. The amplification reaction is performed at 41° C., maintaining constant temperature, typically for about 60 to about 90 minutes (see, e.g., Fakruddin, et al., 2012, “Nucleic Acid Sequence Based Amplification (NASBA) Prospects and Applications,” Int. J. of Life Science and Pharma Res. 2(1):L106-L121).

In some embodiments, the NASBA reaction is carried out at about 40° C. to about 42° C. In some embodiments, the NASBA reaction is carried out at 41° C. In some embodiments, the NASBA reaction is carried out at at most about 42° C. In some embodiments, the NASBA reaction is carried out at about 40° C. to about 41° C., about 40° C. to about 42° C., or about 41° C. to about 42° C. In some embodiments, the NASBA reaction is carried out at about 40° C., about 41° C., or about 42° C.

In some embodiments, the amplification method is Strand Displacement Amplification (SDA). SDA is an isothermal amplification method that uses four different primers. A primer comprising a restriction site (a recognition sequence for HincII exonuclease) is annealed to the DNA template. An exonuclease-deficient fragment of Eschericia coli DNA polymerase 1 (exoKlenow) elongates the primers. Each SDA cycle consists of (1) primer binding to a displaced target fragment, (2) extension of the primer/target complex by exo-Klenow, (3) nicking of the resultant hemiphosphothioate HincII site, (4) dissociation ofHincII from the nicked site and (5) extension of the nick and displacement of the downstream strand by exo-Klenow.

In some embodiments, this disclosure provides for methods comprising contacting DNA in a sample with a helicase. In some embodiments, the amplification method is Helicase Dependent Amplification (HDA). HDA is an isothermal reaction because a helicase, instead of heat, is used to denature DNA.

In some embodiments, the amplification method is Multiple Displacement Amplification (MDA). The MDA is an isothermal, strand-displacing method based on the use of the highly processive and strand-displacing DNA polymerase from bacteriophage 029, in conjunction with modified random primers to amplify the entire genome with high fidelity. It has been developed to amplify all DNA in a sample from a very small amount of starting material. In MDA 029 DNA polymerase is incubated with dNTPs, random hexamers and denatured template DNA at 30° C. for 16 to 18 hours and the enzyme must be inactivated at high temperature (65° C.) for 10 min. No repeated recycling is required, but a short initial denaturation step, the amplification step, and a final inactivation of the enzyme are needed.

In some embodiments, the amplification method is Rolling Circle Amplification (RCA). RCA is an isothermal nucleic acid amplification method which allows amplification of the probe DNA sequences by more than 109 fold at a single temperature, typically about 30° C. Numerous rounds of isothermal enzymatic synthesis are carried out by 029 DNA polymerase, which extends a circle-hybridized primer by continuously progressing around the circular DNA probe. In some embodiments, the amplification reaction is carried out using RCA, at about 28° C. to about 32° C.

Additional amplification methods can be used. Ideally, the amplification method is isothermal and fast relative to traditional PCR. In some embodiments, amplifying comprises performing an exponential amplification reaction (EXP AR), which is an isothermal molecular chain reaction in that the products of one reaction catalyze further reactions that create the same products. In some embodiments, amplifying occurs in the presence of an endonuclease. The endonuclease may be a nicking endonuclease. (Wu et al., “Aligner-Mediated Cleavage of Nucleic Acids,” Chemical Science (2018)). In some embodiments, amplifying does not require initial heat denaturation of target DNA. (Toley et al., “Isothermal strand displacement amplification (iSDA): a rapid and sensitive method of nucleic acid amplification for point-of-care diagnosis,” The Analyst (2015)).

In some embodiments, this disclosure provides for methods comprising performing multiple cycles of nucleic acid amplification with a pair of primers. The number of amplification cycles is important because amplification may introduce a bias into the representation of regions. Not all regions amplify with the same efficiency and therefore the overall representation may not be uniform which will impact the accuracy of the analysis. Fewer amplification cycles are ideal if amplification is necessary at all. In some embodiments, methods comprise performing fewer than 50, 45, 40, 35, 30, or 25 cycles of amplification. In some embodiments, methods comprise performing fewer than 25 cycles of amplification. In some embodiments, methods comprise performing fewer than 20 cycles of amplification. In some embodiments, methods comprise performing fewer than 15 cycles of amplification. In some embodiments, methods comprise performing fewer than 12 cycles of amplification. In some embodiments, methods comprise performing fewer than 11 cycles of amplification. In some embodiments, methods comprise performing fewer than 10 cycles of amplification. In some embodiments, methods comprise performing at least 3 cycles of amplification. In some embodiments, methods comprise performing at least 5 cycles of amplification. In some embodiments, methods comprise performing at least 8 cycles of amplification. In some embodiments, methods comprise performing at least 10 cycles of amplification.

In some embodiments, the amplification reaction is carried for about 30 seconds to about 90 minutes. In some embodiments, the amplification reaction is carried out for at least about 30 minutes. In some embodiments, the amplification reaction is carried out for at most about 90 minutes. In some embodiments, the amplification reaction is carried out for about 30 minutes to about 35 minutes, about 30 minutes to about 40 minutes, about 30 minutes to about 45 minutes, about 30 minutes to about 50 minutes, about 30 minutes to about 55 minutes, about minutes to about 60 minutes, about 30 minutes to about 65 minutes, about 30 minutes to about 70 minutes, about 30 minutes to about 75 minutes, about 30 minutes to about 80 minutes, about 30 minutes to about 90 minutes, about 35 minutes to about 40 minutes, about 35 minutes to about 45 minutes, about 35 minutes to about 50 minutes, about 35 minutes to about 55 minutes, about 35 minutes to about 60 minutes, about 35 minutes to about 65 minutes, about minutes to about 70 minutes, about 35 minutes to about 75 minutes, about 35 minutes to about 80 minutes, about 35 minutes to about 90 minutes, about 40 minutes to about 45 minutes, about 40 minutes to about 50 minutes, about 40 minutes to about 55 minutes, about 40 minutes to about 60 minutes, about 40 minutes to about 65 minutes, about 40 minutes to about 70 minutes, about 40 minutes to about 75 minutes, about 40 minutes to about 80 minutes, about minutes to about 90 minutes, about 45 minutes to about 50 minutes, about 45 minutes to about 55 minutes, about 45 minutes to about 60 minutes, about 45 minutes to about 65 minutes, about 45 minutes to about 70 minutes, about 45 minutes to about 75 minutes, about 45 minutes to about 80 minutes, about 45 minutes to about 90 minutes, about 50 minutes to about 55 minutes, about 50 minutes to about 60 minutes, about 50 minutes to about 65 minutes, about 50 minutes to about 70 minutes, about 50 minutes to about 75 minutes, about 50 minutes to about 80 minutes, about 50 minutes to about 90 minutes, about 55 minutes to about 60 minutes, about 55 minutes to about 65 minutes, about 55 minutes to about 70 minutes, about 55 minutes to about 75 minutes, about 55 minutes to about 80 minutes, about 55 minutes to about 90 minutes, about 60 minutes to about 65 minutes, about 60 minutes to about 70 minutes, about 60 minutes to about 75 minutes, about 60 minutes to about 80 minutes, about 60 minutes to about 90 minutes, about 65 minutes to about 70 minutes, about 65 minutes to about 75 minutes, about 65 minutes to about 80 minutes, about 65 minutes to about 90 minutes, about 70 minutes to about 75 minutes, about 70 minutes to about 80 minutes, about 70 minutes to about 90 minutes, about 75 minutes to about 80 minutes, about 75 minutes to about 90 minutes, or about 80 minutes to about 90 minutes. In some embodiments, the amplification reaction is carried out for about 30 minutes, about 35 minutes, about 40 minutes, about 45 minutes, about 50 minutes, about 55 minutes, about 60 minutes, about 65 minutes, about 70 minutes, about 75 minutes, about 80 minutes, or about 90 minutes.

In some embodiments, this disclosure provides for methods comprising amplifying a nucleic acid at at least one temperature. In some embodiments, this disclosure provides for methods comprising amplifying a nucleic acid at a single temperature (e.g., isothermal amplification). In some embodiments, this disclosure provides for methods comprising amplifying a nucleic acid, wherein the amplifying occurs at not more than two temperatures. Amplifying may occur in one step or multiple steps. Non-limiting examples of amplifying steps include double strand denaturing, primer hybridization, and primer extension.

In some embodiments, at least one step of amplifying occurs at room temperature. In some embodiments, all steps of amplifying occur at room temperature. In some embodiments, at least one step of amplifying occurs in a temperature range. In some embodiments, all steps of amplifying occur in a temperature range. In some embodiments, the temperature range is about 0° C. to about 100° C. In some embodiments, the temperature range is about 15° C. to about 100° C. In some embodiments, the temperature range is about 25° C. to about 100° C. In some embodiments, the temperature range is about 35° C. to about 100° C. In some embodiments, the temperature range is about 55° C. to about 100° C. In some embodiments, the temperature range is about 65° C. to about 100° C. In some embodiments, the temperature range is about 15° C. to about 80° C. In some embodiments, the temperature range is about 25° C. to about 80° C. In some embodiments, the temperature range is about 35° C. to about 80° C. In some embodiments, the temperature range is about 55° C. to about 80° C. In some embodiments, the temperature range is about 65° C. to about 80° C. In some embodiments, the temperature range is about 15° C. to about 60° C. In some embodiments, the temperature range is about 25° C. to about 60° C. In some embodiments, the temperature range is about 35° C. to about 60° C. In some embodiments, the temperature range is about 15° C. to about 40° C. In some embodiments, the temperature range is about −20° C. to about 100° C. In some embodiments, the temperature range is about −20° C. to about 90° C. In some embodiments, the temperature range is about −20° C. to about 50° C. In some embodiments, the temperature range is about −20° C. to about 40° C. In some embodiments, the temperature range is about −20° C. to about 10° C. In some embodiments, the temperature range is about 0° C. to about 100° C. In some embodiments, the temperature range is about 0° C. to about 40° C. In some embodiments, the temperature range is about 0° C. to about 30° C. In some embodiments, the temperature range is about 0° C. to about 20° C. In some embodiments, the temperature range is about 0° C. to about 10° C. In some embodiments, the temperature range is about 15° C. to about 100° C. In some embodiments, the temperature range is about 15° C. to about 90° C. In some embodiments, the temperature range is about 15° C. to about 80° C. In some embodiments, the temperature range is about is about 15° C. to about 70° C. In some embodiments, the temperature range is about 15° C. to about 60° C. In some embodiments, the temperature range is about 15° C. to about 50° C. In some embodiments, the temperature range is about 15° C. to about 30° C. In some embodiments, the temperature range is about 10° C. to about 30° C. In some embodiments, methods disclose herein are performed at room temperature, not requiring cooling, freezing or heating. In some embodiments, amplifying comprises contacting the sample with random oligonucleotide primers.

In some embodiments, amplifying comprises targeted amplification (including that described in U.S. Pat. No. 6,558,928). In some embodiments, amplifying a nucleic acid comprises contacting a nucleic acid with at least one primer having a sequence corresponding to a target gene sequence. In some embodiments, amplifying comprises contacting the nucleic acid with at least one primer having a sequence corresponding to a non-target gene sequence. In some embodiments, amplifying comprises contacting the nucleic acid with not more than one pair of primers, wherein each primer of the pair of primers comprises a sequence corresponding to a sequence on a target gene disclosed herein. In some embodiments, amplifying comprises contacting the nucleic acid with multiple sets of primers, wherein each of a first pair in a first set and each of a pair in a second set are all different.

In some embodiments, amplifying comprises contacting the sample with at least one primer having a sequence corresponding to a sequence on a target gene disclosed herein. In some embodiments, amplifying comprises contacting the sample with at least one primer having a sequence corresponding to a sequence on a non-target gene disclosed herein. In some embodiments, amplifying comprises contacting the sample with not more than one pair of primers, wherein each primer of the pair of primers comprises a sequence corresponding to a sequence on a target gene disclosed herein. In some embodiments, amplifying comprises contacting the sample with multiple sets of primers, wherein each of a first pair in a first set and each of a pair in a second set are all different.

In some embodiments, amplifying comprises multiplexing (nucleic acid amplification of a plurality of nucleic acids in one reaction). In some embodiments, multiplexing comprises contacting nucleic acids of the biological sample with a plurality of oligonucleotide primer pairs. In some embodiments, multiplexing comprising contacting a first nucleic acid and a second nucleic acid, wherein the first nucleic acid corresponds to a first sequence and the second nucleic acid corresponds to a second sequence. In some embodiments, the first sequence and the second sequence are the same. In some embodiments, the first sequence and the second sequence are different. In some embodiments, amplifying does not comprise multiplexing. In some embodiments, amplifying does not require multiplexing. In some embodiments, amplifying comprises nested or hemi-nested or semi-nested primer amplification. Methods may comprise multiplex PCR of multiple regions, wherein each region comprises a SNP. Multiplexing may occur in a single tube. In some embodiments, methods comprise multiplex PCR of more than 100 regions wherein each region comprises a SNP. In some embodiments, methods comprise multiplex PCR of more than 500 regions wherein each region comprises a SNP. In some embodiments, methods comprise multiplex PCR of more than 1000 regions wherein each region comprises a SNP. In some embodiments, methods comprise multiplex PCR of more than 2000 regions wherein each region comprises a SNP. In some embodiments, methods comprise multiplex PCR of more than 300 regions wherein each region comprises a SNP. In some embodiments, amplification can be linear, using a single primer per target, or exponential, using at least one forward and one reverse primer per target.

In some embodiments, this disclosure provides for methods comprising amplifying a nucleic acid in the sample, wherein amplifying comprises contacting the sample with at least one oligonucleotide primer, wherein the at least one oligonucleotide primer is not active or extendable until it is in contact with the sample. In some embodiments, amplifying comprises contacting the sample with at least one oligonucleotide primer, wherein the at least one oligonucleotide primer is not active or extendable until it is exposed to a selected temperature. In some embodiments, amplifying comprises contacting the sample with at least one oligonucleotide primer, wherein the at least one oligonucleotide primer is not active or extendable until it is contacted with an activating reagent. In some embodiments, the at least one oligonucleotide primer comprises a blocking group. Using such oligonucleotide primers may minimize primer dimers, allow recognition of unused primer, and/or avoid false results caused by unused primers. In some embodiments, amplifying comprises contacting the sample with at least one oligonucleotide primer comprising a sequence corresponding to a sequence on a target gene disclosed herein.

In some embodiments, this disclosure provides for methods comprising amplifying nucleic acids in the sample through target-specific amplification, wherein amplifying comprises contacting the sample with primers designed to target selected genes to preferentially enrich and/or amplify relative to other genes within the sample (e.g., relative to a housekeeping gene). In some embodiments, target-specific enrichment involves the use of bait-capture methods, wherein primers functionalized with a biotin can be targeted to selected regions of the genome, and the extended primers can be isolated for further amplification by streptativin beads to produce target-specific amplicons.

In some embodiments, this disclosure provides for methods comprising detecting an amplification product, wherein the amplification product is produced by amplifying at least a portion of a target gene disclosed herein. In some embodiments, detecting amplification products disclosed herein does not comprise tagging or labeling the amplification product. In some embodiments, methods detect the amplification product based on its amount. For example, the methods may detect an increase in the amount of double stranded DNA in the sample. In some embodiments, detecting the amplification product is at least partially based on its size. In some embodiments, the amplification product has a length of about 50 base pairs to about 500 base pairs.

### Library Preparation

In some embodiments, this disclosure provides for methods comprising modifying free nucleic acids from the biological sample to produce a library of nucleic acids for detection. In some embodiments, the free nucleic acids from the biological sample comprise all or a portion of a ADRβ pathway gene. In some embodiments, methods comprise modifying nucleic acids for nucleic acid sequencing. In some embodiments, methods comprise modifying nucleic acids for detection, wherein detection does not comprise nucleic acid sequencing. In some embodiments, methods comprise modifying nucleic acids for detection, to obtain nucleic acids derived from the nucleic acids in the initial sample, wherein detection comprises counting tagged nucleic acids based on an occurrence of tag detection. In some embodiments, this disclosure provides for methods comprising modifying nucleic acids in the biological sample to produce a library of nucleic acids, wherein the method comprises amplifying the nucleic acids. In some embodiments, modifying occurs before amplifying. In some embodiments, modifying occurs after amplifying.

In some embodiments, modifying the nucleic acids comprises repairing ends of nucleic acids that are fragments of a nucleic acid. In some embodiments, repairing ends may comprise restoring a 5′ phosphate group, a 3′ hydroxy group, or a combination thereof to the nucleic acid. In some embodiments, repairing comprises 5′ phosphorylation, A-tailing, gap filling, closing nick sites or a combination thereof. In some embodiments, repairing may comprise removing overhangs. In some embodiments, repairing may comprise filling in overhangs with complementary nucleotides. In some embodiments, modifying the nucleic acids for preparing a library comprises use of an adapter. The adapter may also be referred to herein as a sequencing adapter. In some embodiments, the adapter aids in sequencing. The adapter can comprise an oligonucleotide. In some embodiments, the adapter may simplify other steps in the methods, such as amplifying, purification and sequencing because it is a sequence that is universal to multiple, if not all, nucleic acids in a sample after modifying. In some embodiments, modifying the nucleic acids comprises ligating an adapter to the nucleic acids. Ligating may comprise blunt ligation. In some embodiments, modifying the nucleic acids comprises hybridizing an adapter to the nucleic acids. In some embodiments, the sequencing adapter comprises a hairpin or stem-loop adapter. In some embodiments, modifying the nucleic acids comprises hybridizing a hairpin or stem-loop adapter to the nucleic acids, thereby generating a circular library product that is sequenced or analyzed. In some embodiments, the sequencing adapter comprises a blocked 5′ end leaving a nick at the 3′ end. Advantages of this configuration include, but are not limited to, an increase in library efficiency and reduction of unwanted byproducts such as adapter dimers. In some embodiments the adapter has a cleavable replication stop to linearize templates.

As used herein, the term “tag” refers to a moiety linked to a nucleic acid of interest that can be used as a molecular recognition site to identify or distinguish the nucleic acid in a population, e.g., as a means by which to bioinformatically or physically separate the nucleic acid from the population. A tag can comprise one or more of a number of moieties, including labeled or modified nucleotides, e.g., fluorescently labeled nucleotides, nucleotide analogs, or the like. Tags can also comprise specific nucleotide sequences. In some embodiments, the tags comprise oligonucleotides. In some embodiments, the tags comprise a non-oligonucleotide marker or label that can be detected by means other than nucleic acid analysis. In some embodiments, a non-oligonucleotide marker or label could comprise a fluorescent molecule, a nanoparticle, a dye, a peptide, a metal isotope, or other detectable/quantifiable small molecule.

In some embodiments, modifying the nucleic acids for preparing a library comprises use of a tag. The tag may also be referred to herein as a barcode or barcode oligonucleotide. In some embodiments, a barcode is used to identify individual or subgroups of polynucleotides. In certain embodiments, nucleic acids or other polynucleotides derived from a single strand may share a common tag or identifier and therefore may be later identified as being derived from said strand, permitting parent strand identification. In certain embodiments, the tag may be used to quantify expression, by which the barcode, or barcode combined with the sequence it is attached to can be counted. In some embodiments, the tag may be used as a PCR amplification control wherein multiple amplification products from a PCR can be tagged with the same tag, then if the PCR products are later sequenced and found to have sequence differences, differences among products with the same identifier can then be attributed to PCR error. In some embodiments, this disclosure provides for methods comprising modifying nucleic acids with a tag that corresponds to a chromosomal region of interest. In some embodiments, this disclosure provides for methods comprising modifying nucleic acids with a tag that is specific to a chromosomal region that is not of interest. In some embodiments, this disclosure provides for methods comprising modifying a first portion of nucleic acids with a first tag that corresponds to at least one chromosomal region that is of interest and a second portion of nucleic acids with a second tag that corresponds to at least one chromosomal region that is not of interest. In some embodiments, modifying the nucleic acids comprises ligating a tag to the nucleic acids. Ligating may comprise blunt ligation. In some embodiments, modifying the nucleic acids comprises hybridizing a tag to the nucleic acids. In some embodiments, oligonucleotide primers containing barcode sequences may be used in amplification reactions of the DNA template analytes to produce tagged analytes.

In some embodiments, modifying the nucleic acids for preparing a library comprises use of a sample index, also simply referred to herein as an index. In some embodiments, the index may comprise an oligonucleotide, a small molecule, a fluorescent molecule, a dye, or other detectable/quantifiable moiety. In some embodiments, a first group of nucleic acids from a first biological sample are labeled with a first index, and a first group of nucleic acids from a first biological sample are labeled with a second index, wherein the first index and the second index are different. Thus, multiple indexes allow for distinguishing nucleic acids from multiple samples when multiple samples are analyzed at once. In some embodiments, methods disclose amplifying nucleic acids wherein an oligonucleotide primer used to amplify the nucleic acids comprises an index.

In some embodiments, the methods disclosed herein comprise amplification and library preparation in anticipation of downstream massively parallel sequencing. An assay can include one or two PCR master mixes, one or two thermostable polymerases, one or two primer mixes and library adapters. In some embodiments, a sample of DNA may be amplified for a number of cycles by using a first set of amplification primers that comprise target specific regions and non-target specific tag regions and a first PCR master mix. The tag region can be any sequence, such as a universal tag region, a capture tag region, an amplification tag region, a sequencing tag region, a UMI (unique molecular identifier) tag region, and the like. In some embodiments, a tag region can be the template for amplification primers utilized in a second or subsequent round of amplification, for example for library preparation. In some embodiments, the methods comprise adding single stranded-binding protein to the first amplification products. An aliquot of the first amplified sample can be removed and amplified a second time using a second set of amplification primers that are specific to the tag region, e.g., a universal tag region or an amplification tag region, of the first amplification primers which may comprise of one or more additional tag sequences, such as sequence tags specific for one or more downstream sequencing workflows, and the same or a second PCR master mix. As such, a library of the original DNA sample can be ready for sequencing.

In some embodiments, the free nucleic acids comprising all or a portion of an ADRβ pathway genes is a genomic sample. This disclosure includes methods of producing amplicons from a genomic DNA that can be used to provide templates to a sequencing reaction, e.g., in a high-throughput sequencing system. Genomic DNA can be prepared three steps: cell lysis, deproteinization and recovery of DNA. These steps can be configured to the demands of the application, the requested yield, purity and molecular weight of the DNA, and the amount and history of the source. In addition, many kits are commercially available for the purification of genomic DNA from cells, including Wizard™ Genomic DNA Purification Kit, available from Promega; Aqua Pure™ Genomic DNA Isolation Kit, available from BioRad; Easy-DNA™ Kit, available from Invitrogen; and DnEasy™ Tissue Kit, which is available from Qiagen. In some embodiments, the free nucleic acids comprising all or a portion of an ADRβ pathway genes is an mRNA sample. The mRNA can be converted to cDNA for analysis by DNA sequencing using the methods described herein. Analysis of cDNA affords detection of alternative splicing. Data obtained from sequencing cDNAs can be useful in identifying novel splice variants of a selected gene of interest and/or in comparing the differential expression of splice isoforms of a selected gene of interest, e.g., between different tissue types, between different treatments to the same tissue type or between different developmental stages of the same tissue type. The methods for preparing amplicons that are provided herein can be beneficially used to produce templates derived from cDNAs to high throughput sequencing systems. cDNAs are prepared from mRNA. mRNA can typically be isolated from almost any source using protocols and methods described in, e.g., Sambrook and Ausubel. In some embodiments, the mRNA can be isolated using a commercialliy available mRNA isolation kit, e.g., the mRNA-ONLY™ Prokaryotic mRNA Isolation Kit and the mRNA-ONLY™ Eukaryotic mRNA Isolation Kit (Epicentre Biotechnologies), the FastTrack 2.0 mRNA Isolation Kit (Invitrogen), and the Easy-mRNA Kit (BioChain). The purified mRNA can be reverse transcribed using reverse transcriptase to generate cDNAs from the mRNA templates. Methods and protocols for the production of cDNA from mRNAs, are elaborated in cDNA Library Protocols, I. G. Cowell, et al., eds., Humana Press, New Jersey, 1997, Sambrook and Ausubel. In addition, many kits are commercially available for the preparation of cDNA, including the Cells-to-cDNA™ II Kit (Ambion), the RETROscript™ Kit (Ambion), the CloneMiner™ cDNA Library Construction Kit (Invitrogen), and the Universal RiboClone® cDNA Synthesis System (Promega). Many companies, e.g., Agencourt Bioscience and Clontech, offer cDNA synthesis services.

### Sequencing

The methods provided herein are useful for the preparation of cell-free polynucleotide sequences for downstream sequencing reactions. In certain embodiments, the method of preparing gene amplicons or enriched nucleic acid samples further comprises detecting the clonally amplified gene amplicons by multiplex PCR or sequencing.

In certain embodiments, clonally amplified gene amplicons are detected using, for example, polymerase chain reaction (PCR), multiplex PCR, or the Luminex assay (Bio-techne, Minneapolis, MN, USA). In some embodiments, clonally amplified gene amplicons are detected using sequencing. The sequencing methods can include or exclude massively parallel sequencing (e.g., SBS), nanopore sequencing, or pyrosequencing. In some embodiments, the sequencing can be targeted or random sequencing.

In some embodiments, this disclosure provides for methods comprising sequencing a nucleic acid. In some embodiments, the nucleic acid is obtained from a sample or nucleic acids derived therefrom. In some embodiments, the nucleic acid is a recombinant DNA expressing a gene or target of interest. The nucleic acid may be a nucleic acid disclosed herein, such as an amplified nucleic acid. In some embodiments the nucleic acid is DNA. In some embodiments, the nucleic acid is RNA. In some embodiments, the DNA is selected from the group consisting of circulating DNA (cf-DNA), genomic DNA (gDNA), mitochondrial DNA, and pathogenic DNA (e.g., viral genomic DNA (vgDNA), fungal DNA, bacterial DNA). In some embodiments, the nucleic acid is RNA (e.g., cf-RNA). In some embodiments, the nucleic acid is in the form of complementary DNA (cDNA), generated by reverse transcription of a cf-RNA or cff-RNA. In some embodiments, the cf-RNA or cff-RNA is a messenger RNA (mRNA), a microRNA (miRNA), mitochondrial RNA, or a natural antisense RNA (NAS-RNA). In some embodiments, the nucleic acid sequence comprises an RNA molecule or a fragmented RNA molecule (RNA fragments) selected from: small interfering RNA (siRNA), a microRNA (miRNA), a premiRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a RNA, an exosomal RNA, a vector-expressed RNA, an RNA transcript, and combinations thereof. In some embodiments, the nucleic acid is a fetal nucleic acid, a newborn nucleic acid (obtained from a subject having an age less than 3, 2, or 1 years) a nucleic acid having a sequence corresponding to a target gene, a nucleic acid having a sequence corresponding to a region of a target gene, a nucleic acid having a sequence corresponding to a non-target gene, or a combination thereof.

Sequencing methods may include, but are not limited to: targeted sequencing, high-throughput sequencing, pyrosequencing, sequencing-by-synthesis, sequencing-by-hybdridization, RNA-Seq, Digital Gene Expression, Next generation sequencing, massively-parallel sequencing, shotgun sequencing, semiconductor sequencing, sequencing-by-ligation, Maxim-Gilbert sequencing, primer walking, single molecule sequencing by synthesis, clonal single molecule array, sequencing using PacBio, SOLID, Ion Torrent, or Nanopore platforms. The present methods are not limited to any particular sequencing platform.

In some embodiments, sequencing comprises targeted sequencing. In some embodiments, sequencing comprises whole genome sequencing. In some embodiments, sequencing comprises targeted sequencing and whole genome sequencing. In some embodiments, whole genome sequencing comprises massive parallel sequencing. In some embodiments, whole genome sequencing comprises random massive parallel sequencing. In some embodiments, sequencing comprises random massive parallel sequencing of target regions captured from a whole genome library.

In some embodiments, the methods comprise sequencing amplified nucleic acids. In some embodiments, amplified nucleic acids are produced by targeted amplification (e.g., with primers specific to target sequences of interest, or pulldown capture (e.g., bait) sequences first isolating selected regions of a genome followed by the amplification of said pulled-down regions). In some embodiments, amplified nucleic acids are produced by non-targeted amplification (e.g., with random oligonucleotide primers). In some embodiments, methods comprise sequencing amplified nucleic acids, wherein the sequencing comprises massive parallel sequencing.

In some embodiments, the sequencing can be performed by sequencing by synthesis (“SBS”), a type of massively parallel sequencing. In some embodiments, massively parallel sequencing comprises a flow cell wherein nucleic acids are attached at (or through a hydrogel to) fixed locations in an array such that their relative positions do not change and wherein the array is repeatedly imaged. Examples in which images are obtained in different color channels, for example, coinciding with different labels used to distinguish one nucleotide base type from another are particularly applicable.

In some embodiments, SBS methods comprise the enzymatic extension of a priming nucleic acid strand through the iterative addition of (optionally labelled-and-3′ blocked) nucleotides against a template strand. In certain SBS methods, a single nucleotide monomer may be provided to a target nucleotide in the presence of a polymerase in each delivery. However, in the methods described herein, more than one type of nucleotide monomer can be provided to a target nucleic acid in the presence of a delivered polymerase.

In some embodiments, SBS methods involve nucleotide monomers that comprise a label moiety. In some embodiments, SBS methods involve nucleotide monomers that no not have a label moiety. Nucleotide incorporation events can be detected based on a characteristic of the label, such as fluorescence of the label; a characteristic of the nucleotide monomer such as charge; a byproduct of incorporation of the nucleotide, such as release of pyrophosphate; or the like. In some embodiments, two or more nucleotides are present in a sequencing step, and the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods presently commercialized by Illumina, Inc. (San Diego, CA), Element Biosciences, or Singular Genomics.

In some embodiments, SBS involves pyrosequencing. Pyrosequencing detects the release of inorganic pyrophosphate (PPi) or protons as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing methodbased on real-time pyrophosphate.” Science 281(5375), 363; U.S. Pat. No. 6,210,891; the disclosures of which are incorporated herein by reference in their entireties).

The pyrosequencing mechanism involves detecting released PPi by being enzymatically converted to adenosine triphosphate (ATP) by ATP sulfurylase, such that the amount of ATP generated is detected via luciferase-produced photons. The nucleic acids to be sequenced can be attached to features in an array comprising wells to localize the released ATP and reduce or prevent ATP crossover from well to well, and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. The relative locations of each feature, however, will remain unchanged in the images. The images can be stored, processed and analyzed. In some embodiments, images obtained after treatment of the array with each different nucleotide type can be handled in the same way as for images obtained from different detection channels for reversible terminator-based sequencing methods.

In some embodiments, SBS methods involve detection of a proton released upon incorporation of a nucleotide into an extension product. For example, sequencing based on detection of released protons can use an electrical detector and associated techniques that are commercially available from ThermoFisher Scientific as the Ion Torrent instrument or sequencing methods and systems described in U.S. Pat. No. 8,262,900A1, incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

In some embodiments, SBS involves cycle sequencing by the stepwise addition of reversible terminator nucleotides which comprise a cleavable or photobleachable dye label (as described in U.S. Pat. No. 7,057,026, the disclosure of which is incorporated herein by reference). This approach is commercialized Illumina Inc., and is also described in WO 91/06678 (also referred to as U.S. Pat. No. 427,321, Tsien et al.) and U.S. Pat. No. 8,241,573, each of which is incorporated herein by reference. The availability of fluorescently-labeled terminators in which both the termination can be reversed and the fluorescent label cleaved facilitates efficient cyclic reversible termination sequencing. Polymerases can also be engineered to efficiently incorporate and extend from these modified nucleotides. Additional exemplary SBS systems and methods which can be utilized with the methods and systems described herein are described in U.S. Pat. Nos. 7,541,444, 7,566,537, 7,057,026, 8,460,910, 8,623,628, 8,951,781, and 9,193,996, the disclosures of which are incorporated herein by reference in their entireties.

For cluster generation, the library fragments are immobilized on a substrate, for example a slide, which comprises homologous oligonucleotide sequences for capturing and immobilizing the DNA library fragments. The immobilized DNA library fragments are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, preferably via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized. However, the methods described herein are not limited to any particular sequencing preparation methodology or sequencing platform and can be amenable to other parallel sequencing platform preparation methods and associated sequencing platforms.

In some embodiments, SBS methods involve detection of four different nucleotides using fewer than four different labels. SBS can be performed utilizing methods and systems described in the incorporated materials of U.S. Pat. No. 9,453,258. In some embodiments, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. In some embodiments, three of four different nucleotide types can be detected under particular conditions while a fourth nucleotide type lacks a label that is detectable under those conditions, or is minimally detected under those conditions (e.g., due to background fluorescence). Incorporation of the first three nucleotide types into a nucleic acid can be determined based on presence of their respective signals and incorporation of the fourth nucleotide type into the nucleic acid can be determined based on absence or minimal detection of any signal. In some embodiments, one nucleotide type can include label(s) that are detected in two different channels, whereas other nucleotide types are detected in no more than one of the channels. The aforementioned three exemplary configurations are not considered mutually exclusive and can be used in various combinations. In some embodiments, SBS methods can involve a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pat. No. 9,453,258, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

In some embodiments, massively parallel sequencing can be performed by methods capable of single molecule sequencing, with or without amplification or labeling, such as nanopore sequencing (as described in: Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Ace. Chem. Res. 35:817-825 (2002); Li, J., M. Gershow, D. Stein, E. Brandin, and J. A Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as alpha-hemolysin. Each base-pair can be identified by measuring fluctuations in the electrical conductance of the pore as the target nucleic acid passes through the nanopore. (U.S. Pat. No. 7,001,792; Soni, G. V. & Meller, “A Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties).

In some embodiments, sequencing can be performed by methods comprising the real-time monitoring of DNA polymerase activity. Nucleotide incorporations can be detected through fluorescence resonance energy transfer (FRET) interactions between a fluorophore-bearing polymerase and gamma-phosphate-labeled nucleotides as described, for example, in U.S. Pat. No. 7,329,492 (which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. Nos. 7,405,281 and 8,343,746 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); the disclosures of which are incorporated herein by reference in their entireties). Commercial systems employing such technologies are presently commercialized by PacBio (Menlo Park, CA, USA).

The massively parallel sequencing methods described herein can be advantageously carried out in multiplex formats such that multiple different target nucleic acids are manipulated simultaneously. In some embodiments, different target nucleic acids can be treated in a common reaction vessel or on a surface of a particular substrate, allowing for the convenient delivery of sequencing reagents, removal of unreacted reagents and detection of incorporation events in a multiplexed manner. In embodiments involving surface-bound (or bound through a hydroglel to a surface) target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. In some embodiments, the target nucleic acids can be bound through a surface-immobilized hydrogel (as described in U.S. Pat. No. 9,012,022, incorporated herein by reference). The array can include a single copy of a target nucleic acid at each site (also referred to as a feature) or multiple copies having the same sequence can be present at each site or feature (also referred to as a colony). Multiple copies can be produced by amplification methods which can include or exclude bridge amplification or emulsion PCR as described in further detail below.

The methods of the present disclosure utilize the Illumina, Inc. SBS technology for sequencing the DNA profile libraries created by practicing the methods described herein. The Novaseq 6000 platform (a sequencing instrument) was used for clustering and sequencing for the examples described herein. However, as understood by a skilled artisan, the present methods are not limited by the type of sequencing platform used.

In some embodiments, sequencing libraries are prepared prior to sequencing. Sequencing library preparation involves the production of a random collection of adapter-modified DNA fragments, which are ready to be sequenced. Sequencing libraries of polynucleotides can be prepared from DNA or RNA, including equivalents, analogs of either DNA or cDNA, that is complementary or copy DNA produced from an RNA template, for example by the action of reverse transcriptase. The polynucleotides may originate in double-stranded DNA (dsDNA) form (e.g. genomic DNA fragments, PCR and amplification products) or polynucleotides that may have originated in single-stranded form, as DNA or RNA, and been converted to dsDNA form. By way of example, mRNA molecules may be copied into double-stranded cDNAs suitable for use in preparing a sequencing library. The precise sequence of the primary polynucleotide molecules is generally not material to the method of library preparation, and may be known or unknown. Preparation of sequencing libraries for some sequencing platforms may require that the polynucleotides be or a specific range of fragment sizes e.g. 0-1200 bp. Therefore, fragmentation of polynucleotides e.g. genomic DNA may be required.

In some embodiments, the sequencing reactions herein may comprise a variety of sample processing units, including but not limited to: multiple lanes, multiple channels, multiple wells, multiple droplets, multiple sites on a flow cell or other solid substrate, or other means of processing multiple sample sets simultaneously. In some embodiments, simultaneous sequencing reactions may be performed using multiplex sequencing. In some embodiments, sequencing reactions may be performed sequentially or simultaneously.

In certain embodiments, polynucleotides may be sequenced with at least 1000, 2000, 3000, 4000, 5000, 6000, 7000, 8000, 9000, 10000, 50000, 100,000 sequencing reactions. In certain other embodiments, polynucleotides may be sequenced with less than 1000, 2000, 3000, 4000, 5000, 6000, 7000, 8000, 9000, 10000, 50000, 100,000 sequencing reactions. Subsequent data analysis may be performed on all or part of the sequencing reactions. In some embodiments, data analysis may be performed on at least 1000, 2000, 3000, 4000, 5000, 6000, 7000, 8000, 9000, 10000, 50000, 100,000 sequencing reactions. In certain other embodiments, data analysis may be performed on less than 1000, 2000, 3000, 4000, 5000, 6000, 7000, 8000, 9000, 10000, 50000, 100,000 sequencing reactions. In some embodiments, the number of sequence reactions may provide coverage for different amounts of the genome. In certain embodiments, sequence coverage of the genome may be at least 5%, 10%, 15%, 20%, 25%, 30%, 40%, 50%, 60%, 70%, 80%, 90%, 95%, 99%, 99.9% or 100%. In other certain embodiments, sequence coverage of the genome may be less than 5%, 10%, 15%, 20%, 25%, 30%, 40%, 50%, 60%, 70%, 80%, 90%, 95%, 99%, 99.9% or 100%.

Standard protocols e.g. protocols for sequencing using, for example, the Illumina platforms, instruct users to purify the end-repaired products prior to dA-tailing, and to purify the dA-tailing products prior to the adapter-ligating steps of the library preparation. Purification of the end-repaired products and dA-tailed products remove enzymes, buffers, salts and the like to provide favorable reaction conditions for the subsequent enzymatic step. In one embodiment, the steps of end-repairing, dA-tailing and adapter ligating exclude the purification steps. Thus, in one embodiment, the method of the invention encompasses preparing a sequencing library that comprises the consecutive steps of end-repairing, dA-tailing and adapter-ligating.

As part of the sequencing protocol, an amplification reaction is prepared. The amplification step introduces to the adapter ligated template molecules the oligonucleotide sequences required for hybridization to the flow cell. The contents of an amplification reaction include appropriate substrates (such as dNTPs), enzymes (e.g. a DNA polymerase) and buffer components required for an amplification reaction. Optionally, amplification of adapter-ligated polynucleotides can be omitted. Generally, amplification reactions require at least two amplification primers i.e. primer oligonucleotides, which may be identical, and include an-adapter-specific portion, capable of annealing to a primer-binding sequence in the polynucleotide molecule to be amplified (or the complement thereof if the template is viewed as a single strand) during the annealing step. Once formed, the library or templates prepared according to the methods described above can be used for solid-phase nucleic acid amplification. The term ‘solid-phase amplification’ as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, wherein one primer is anchored to a bead and the other is in free solution, and colony formation in solid phase gel matrices wherein one primer is anchored to the surface, and one is in free solution. The library of template polynucleotide can be used in sequencing methods. In addition to providing templates for solid-phase sequencing and solid-phase PCR, library templates provide templates for whole genome amplification.

Sequencing of the amplified libraries can be carried out using any suitable sequencing technique as described herein. In some embodiments, sequencing is performed by massively parallel sequencing using sequencing-by-synthesis (SBS), or single molecule sequencing such as nanopore sequencing.

In some embodiments, this disclosure provides for methods which employ massively parallel sequencing technology in which clonally amplified DNA templates or single DNA molecules are sequenced in a massively parallel fashion within a flow cell. Massively parallel sequencing provides digital quantitative information, in that each sequence read is a countable “sequence tag” representing an individual clonal DNA template or a single DNA molecule. This quantification allows sequencing to expand the digital PCR concept of counting DNA molecules (Fan et al., Proc Natl Acad Sci USA 105.16266-16271 [2008]; Chiu et al., Proc Natl Acad Sci USA 2008; 105:20458-20463 (2008).

In one embodiment, the method employs massively parallel sequencing of millions of DNA fragments using Illumina's sequencing-by-synthesis (SBS) and reversible terminator-based sequencing chemistry, as described herein. In some embodiments, template DNA can be genomic DNA e.g. ctDNA. In some embodiments, genomic DNA from isolated cells is used as the template, and is fragmented into lengths of several hundred base pairs. Illumina's sequencing technology involves the attachment of fragmented genomic DNA to a planar, optically transparent surface on which oligonucleotide anchors are bound to a surface-immobilized hydrogel polymer. Template DNA is end-repaired to generate 5′-phosphorylated blunt ends, and the polymerase activity of Klenow fragment is used to add a single A base to the 3′ end of the blunt phosphorylated DNA fragments. The addition prepares the DNA fragments for ligation to oligonucleotide adapters, which have an overhang of a single T base at their 3′ end to increase ligation efficiency. The adapter oligonucleotides are complementary to the flow-cell anchors. Under limiting-dilution conditions, adapter-modified, single-stranded template DNA is added to the flow cell and immobilized by hybridization to the anchors. Attached DNA fragments are extended and bridge amplified to create an ultra-high density sequencing flow cell with hundreds of millions of clusters, each containing ˜1,000 copies of the same template.

In some embodiments, this disclosure provides for a method of amplifying target loci in a nucleic acid sample, the method comprising:
a. performing multiplex PCR on a nucleic acid sample comprising target loci to simultaneously amplify at least 2 distinct target loci using either (i) at least 2 different primer pairs or (ii) at least 2 target-specific primers and a universal or tag-specific primer, wherein the multiplex PCR is performed in a single reaction volume to produce amplified products comprising target amplicons. In some embodiments, the method further comprises the step of:
b. sequencing the amplified products using massively parallel sequencing; wherein the target loci are selected from the gene mutations found in the group selected from: rs2240079, rs11168215, rs11168214, rs61917617, rs55683248, rs2072341, rs8082254, rs72847785, rs3181252, and rs17256902.

In some embodiments, this disclosure provides for a method of sequencing at least a portion of a population of sample nucleic acids, wherein the sample nucleic acids are derived from the genome of an organism, wherein the method comprises:
a. forming a reaction mixture comprising the population of sample nucleic acids and a set of unique molecular identifier tags (UMIs), wherein the UMIs are nucleic acids, wherein the number of different UMIs in the set of UMIs is between 10 and 1,000, wherein the UMIs are between 4 and 18 nucleotides in length and wherein the sequence of each of the UMIs in the set of UMIs differs from all other UMI sequences in the set by at least 2 nucleotides, wherein the diversity of combinations of any 2 UMIs in the set of UMIs exceeds the total number of sample nucleic acids that span each target locus, and wherein a ratio of the total number of sample nucleic acids in the population of sample nucleic acids to the number of different UMIs in the set of UMIs is at least 1,000:1;
b. attaching at least one UMI from the set of UMIs to a sample nucleic acid molecule or segment thereof for at least 50% of the sample nucleic acids to form a population of tagged nucleic acids, wherein the at least one UMI is located 5′ and/or 3′ to the sample nucleic acid molecule or segment thereof on each tagged nucleic acid molecule and wherein the population of tagged nucleic acids comprises at least one copy of each UMI of the set of UMIs;
c. amplifying the population of tagged nucleic acids to create a library of tagged nucleic acids; and
d. determining the sequences of at least a portion of the tagged nucleic acids by massively parallel sequencing,
wherein each target locus is selected from the group consisting of: rs2240079, rs11168215, rs11168214, rs61917617, rs55683248, rs2072341, rs8082254, rs72847785, rs3181252, and rs17256902.

In some embodiments, the method further comprises the steps of:
e. ligating at least one adapter to the sample nucleic acids, wherein the at least one adapter comprises a universal amplification sequence;
f. performing universal amplification using universal primers that bind to the universal amplification sequence to generate amplified nucleic acids;
g. performing clonal amplification of the amplified nucleic acids to generate clonally amplified nucleic acids; and
h. measuring the amounts of clonally amplified nucleic acids by performing massively parallel sequencing,
wherein said sample nucleic acids includes a target locus is selected from the group consisting of: rs2240079, rs11168215, rs11168214, rs61917617, rs55683248, rs2072341, rs8082254, rs72847785, rs3181252, and rs17256902.

In some embodiments, the methods comprise generating a library from the nucleic acids in the biological sample, e.g., a library of nucleic acids, adapter-nucleic acids and/or enriched nucleic acids or enriched adapter-nucleic acids, e.g., bait enriched or amplification products with or without adapters. In some embodiments, the methods comprise determining the sequences of the nucleic acid library. In some embodiments, the nucleic acid sample is obtained or derived from a sample obtained from a human.

In some embodiments, each of the plurality of primers comprises one or more tag sequences. In some embodiments, the one or more tag sequences comprise a primer tag, a capture tag, a sequencing tag, a unique molecular identifier tag, or a combination thereof. In some embodiments, the one or more tag sequences comprise a primer tag. In some embodiments, the one or more tag sequences comprise a unique molecular identifier (UMI) tag.

In some embodiments, nucleic acid sequencing may comprise sequencing at least about 10, 20, 30, 40, 50, 60, 70, 80, 90, 100, 110, 120, 130, 140, 150, 160, 170, 180, 190,200, 210, 220, 230, 240, 250, 260, 270, 280, 290, 300 or more nucleotides or base pairs of the nucleic acid molecule sequences. In some embodiments, sequencing may comprise sequencing at least about 200, 300, 400, 500, 600, 700, 800, 900, 1,000 or more nucleotides or base pairs of the nucleic acid molecule sequences. In other embodiments, sequencing may comprise sequencing at least about 1,500; 2,000; 3,000; 4,000; 5,000; 6,000; 7,000; 8,000; 9,000; 10,000; 50,000 or more nucleotides or base pairs of the nucleic acid molecule sequences.

In some embodiments, nucleic acid sequencing may comprise at least about 200, 300, 400, 500, 600, 700, 800, 900, 1,000 or more sequencing reads per run. In some embodiments, sequencing may comprise sequencing at least about 1,500; 2,000; 3,000; 4,000; 5,000; 6,000; 7,000; 8,000; 9,000; or 10,000 or more sequencing reads per run. In some embodiments, nucleic acid sequencing may comprise at least about 10,000; 20,000; 30,000; 40,000; 50,000; 60,000; 70,000; 80,000; 90,000; or 100,000 or more sequencing reads per run. In some embodiments, nucleic acid sequencing may comprise at least about 250,000; 500,000; 1,000,000; 10,000,000; 100,000,000; or 1,000,000,000 or more sequencing reads per run. In some embodiments, nucleic acid sequencing may comprise less than or equal to about 1,600,000,000 sequencing reads per run. In some embodiments, nucleic acid sequencing may comprise less than or equal to about 200,000,000 reads per run.

The number of times a single nucleotide or polynucleotide is identified or “read” is defined as the sequencing depth or read depth, or fold coverage, optionally describing a percentage of bases. Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of percentage coverage (or coverage breadth). For example, 10 million reads of a 1 million base genome, perfectly distributed, theoretically results in 10× read depth of 100% of the sequences. In practice, a greater number of reads (higher theoretical read depth, or oversampling) may be needed to obtain the desired read depth for a percentage of the target sequences.

In some embodiments, sequencing is performed at a read depth of at least 10, 15, 20, 25, 30, 35, 40, 45, 50, 55, 60, 65, 70, 75, 80, 85, 90, 95, 100, 150, 200, 250, 300, 350, 400, 450, 500, 550. 600, 650, 700, 750, 800, 850, 900, 950, or at least 1000, at least 10,000, at least 20,000, at least 30,000, at least 50,0000, at least 75,000, or least 100,000 unique reads per base. In some embodiments, sequencing may comprise a read depth of at least 1×, 5×, 10×, 20×, 30×, 40×, 50×, 60×, 70×, 80×, 90×, 100×, or more.

Enrichment of target sequences with a controlled stoichiometry probe library increases the efficiency of downstream sequencing, as fewer total reads will be required to obtain an outcome with an acceptable number of reads over a desired % of target sequences. For example, in some instances, 55× theoretical read depth of target sequences results in at least 30× coverage of at least 90% of the sequences. In some instances, no more than 55× theoretical read depth of target sequences results in at least 30× read depth of at least 80% of the sequences. In some instances, no more than 55× theoretical read depth of target sequences results in at least 30× read depth of at least 95% of the sequences. In some instances, no more than 55× theoretical read depth of target sequences results in at least 10× read depth of at least 98% of the sequences. In some instances, 55× theoretical read depth of target sequences results in at least 20× read depth of at least 98% of the sequences. In some instances, no more than 55× theoretical read depth of target sequences results in at least 5× read depth of at least 98% of the sequences. Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5×. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

In some embodiments, RAPGEF3 gene amplicons are obtained by using a forward primer having 80% homology or higher to that of the nucleotide sequence 5′ CTTCCTTCATTTCTCCACCTG 3′ (SEQ ID NO: 1) and the reverse primer having 80% homology or higher to that of the nucleotide sequence 5′ TCTGTGTCCTCTTGCCTGC 3′ (SEQ ID NO: 2).

Identity or homology with respect to a specified amino acid sequence of this invention is defined herein as the percentage of amino acid residues in a candidate sequence that are identical with the specified residues, after aligning the sequences and introducing gaps, if necessary, to achieve the maximum percent homology, and not considering any conservative substitutions as part of the sequence identity. None of N-terminal, C-terminal or internal extensions, deletions, or insertions into the specified sequence shall be construed as affecting homology. All sequence alignments called for in this invention are such maximal homology alignments. Generally, the nucleic acid sequence homology between the polynucleotides, oligonucleotides, and fragments of the invention and a nucleic acid sequence of interest will be at least 80% or greater, and more typically with preferably increasing homologies of at least 85%, 90%, 91%, 92%, 92%, 94%, 95%, 96%, 97%, 98%, 99%, and/or 100%. Two amino acid sequences are homologous if there is a partial or complete identity between their sequences.

In some embodiments, the practice of the present disclosure will employ, together with the methods featured herein, unless otherwise indicated molecular biology, microbiology, recombinant DNA, and immunology techniques. See, e.g., Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane (Cold Spring Harbor Laboratory Press, 1988); and Handbook Of Experimental Immunology, Volumes I-IV (D. M. Weir and C. C. Blackwell, eds., 1986).

### Polymorphic Sequences

Polymorphic sites that are contained in the target nucleic acids include without limitation single nucleotide polymorphisms (SNPs), tandem SNPs, small-scale multi-base deletions or insertions, also referred to as “IN-DELS” or deletion insertion polymorphisms “DIPs”, Multi-Nucleotide Polymorphisms “MNPs”, and Short Tandem Repeats “STRs”.

In one embodiment, the nucleic acids in the sample is enriched for target nucleic acids that comprise at least one SNP. In some embodiments, each target nucleic acid comprises a single i.e. one SNP. Target nucleic acid sequences comprising SNPs are available from publically accessible databases including, but not limited to Human SNP Database at world wide web address wi.mit.edu, NCBI dbSNP Home Page at world wide web address ncbi.nlm.nih.gov, world wide web address lifesciences.perkinelmer.com, Celera Human SNP database at world wide web address celera.com, the SNP Database of the Genome Anulysis Group (GAN) at world wide web address gan.iarc.fr. In one embodiment, the SNPs chosen selected from the group of 92 individual identification SNPs (IISNPs) described by Pakstis el al. (Pakstis et el. Hum Genet 127:315-324 [2010]), which have been shown to have a very small variation in frequency across populations (Fₛₜ<0.06), and to be highly in formative around the world having an average heterozygosity ≥0.4. SNPs that are encompassed by the method of the invention include linked and unlinked SNPs. Each target nucleic acid comprises at least one polymorphic site e.g. a single SNP, that differs from that present on another target nucleic acid to generate a panel of polymorphic sites e.g. SNPs, that contain a sufficient number of polymorphic sites of which at least 1, at least 2, at least 3, at least 4, at least 5, at least 6, at least 7, at least 8, a.t least 9, at least 10, at least 11, at least 12, at least 13, at least 14, at least 15, at least 16, at least 17, at least 18, at least 19, at least 20, at least 25, at least 30, at least 35, at least 40 or more are informative. For example, a panel of SNPs can be configured. to comprise at. least one informative SNP.

In one embodiment, the SNPs that are targeted for amplification include, but are not limited to, RAPGEF3, ADCY7, ADCY9, PRKAR1A, and ADCY4.

Enrichment of the sample for the target nucleic acids is accomplished by methods that comprise specifically amplifying target nucleic acid sequences that comprise the polymorphic site. Amplification of the target sequences can be performed by any method that uses PCR or variations of the method including but not limited to asymmetric PCR, helicase-dependent amplification, hot-start PCR, qPCR, solid phase PCR, and touchdown PCR. Alternatively, replication of target nucleic acid sequences can be obtained by enzyme-independent methods e.g. chemical solid-phase synthesis using the phosphoramidites. Amplification of the target sequences is accomplished using primer pairs each capable of amplifying a target nucleic acid sequence comprising the polymorphic site e.g. SNP, in a multiplex PCR reaction. Multiplex PCR reactions include combining at least 2, at least three, at least 3, at least 5, at least 10, at least 15, at least 20, at least 25, at least 30 at least 30, at least 35, at least 40 or more sets of primers in the same reaction to quantify the amplified target nucleic acids comprising at least two, at least three, at least 5, at least 10, at least 15, at least 20, at least 25, at least 30, at least 30, at least 35, at least 40 or more polymorphic sites in the same sequencing reaction. Any panel of primer sets can be configured to amplify—at least one informative polymorphic sequence.

### Amplification of Polymorphic Sequences

Amplification of the target nucleic acids is performed using sequence-specific primers that allow for sequence specific amplification. For example, the PCR primers are designed to discriminate against the amplification of similar genes or paralogs that are on other chromosomes by taking advantage of sequence differences between the target nucleic acid and any paralogs from other chromosomes. The forward or reverse PCR primers are designed to anneal close to the SNP site and to amplify a nucleic acid sequence of sufficient length to be encompassed in the reads generated by massively parallel sequencing methods. Some massively parallel sequencing methods require that nucleic acid sequence have a minimum length (bp) to enable bridging amplification that may optionally be used prior to sequencing. Thus the PCR primers used for amplifying target nucleic acids are designed to amplify sequences that are of sufficient length to the bridge amplified and to identify SNPs that are encompassed by the sequence reads. In some embodiments the first of two primers in the primer set comprising the forward and the reverse primer for amplifying the target nucleic acid is designed to identify a single SNP present within a sequence read of about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. It is expected that technological advances in massively parallel sequencing technologies will enable single-end reads of greater than 500 bp. In one embodiment, one of the PCR primers is designed to amplify SNPs that are encompassed in sequence reads of 36 bp. The second primer is designed to amplify the target nucleic acid as an amplicon of sufficient length to allow for bridge amplification. In one embodiment, the exemplary PCR primers are designed to amplify target nucleic acids that contain a single SNP selected from SNPs rs8082254, rs72847785, rs3181254, rs17256902, rs2240079, rs11168215, rs61917617, rs11168214, rs55683248, and rs2072341. In other embodiments, the forward and reverse primers are each designed for amplifying target nucleic acids each comprising a set of two tandem SNPs, each being present within a sequence read or about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. In one embodiment, at least one of the primers is designed to amplify the target nucleic acid comprising a set of two tandem SNPs as an amplicon of sufficient length to allow for bridge amplification. In some embodiments, target enrichment is performed before amplification resulting in target-specific amplification.

The SNPs, single or tandem SNPs, are contained in amplified target nucleic acid amplicons of at least about 100 bp, at least about 150 bp, at least about 200 bp, at least about 250 bp, at least about 300 bp, at least about 350 bp, or at least about 400 bp. In one embodiment, target nucleic acids comprising a polymorphic site e.g. a SNP, are amplified as amplicons of at least about 110 bp, and that comprise a SNP within 36 bp from the 3′ or 5′ end of the amplicon. In another embodiment, target nucleic acids comprising two or more polymorphic sites e.g. two tandem SNPs, are amplified as amplicons of at least about 110 bp, and that comprise the first SNP within 36 bp from the 3′ end of the amplicon, and/or the second SNP within 36 bp from the 5′ end of the amplicon.

### Detection of Variant Sequences

The detection of mutations including polymorphisms, deletions and insertions utilizes comparison of sequence coverage to a control or reference sequence, and may be performed on selectively enriched regions of the genome or transcriptome. In some embodiments, the detection of mutations is performed in sequence reads that include, but are not limited to, RAPGEF3, ADCY7, ADCY9, PRKAR1A, ADCY4, and genes in the beta-1, beta-2, and beta-3 adrenergic receptors. In some embodiments, sequence reads may contain barcode information. In some embodiments barcodes may be used to distinguish original Watson and Crick strands derived from sample fragments, to allow for error corrected sequencing. In some embodiments, barcodes are not utilized. A reference sequence is obtained from a control sample, taken from another subject. In some cases, the control subject may be a subject known to not have known genetic aberrations or disease.

After sequencing, sequence reads are assigned a quality score which may be a representation of reads that indicates whether said reads may be useful in subsequent analysis based on a threshold. In certain embodiments, some reads are not of sufficient quality or length to perform the subsequent mapping step. Sequencing reads with a quality score at least 90%, 95%, 99%, 99.9%, 99.99% or 99.999% may be filtered out of the data set. In other embodiments, sequencing reads assigned a quality scored at least 90%, 95%, 99%, 99.9%, 99.99% or 99.999% may be filtered out of the data set. Sequence reads that meet a specified quality score threshold are mapped to a reference sequence that is known not to contain rare mutations in genes or sequences of interest.

After mapping alignment, sequence reads are assigned a mapping score. The mapping score may be a representation of reads mapped back to the reference sequence indicating whether each position is or is not uniquely mappable. In some embodiments, reads may be sequences related to mutation analysis. In some embodiments, reads may be sequences unrelated to mutation analysis, for example, some sequence reads may originate from contaminant polynucleotides. For each mappable base, bases that do not meet the minimum threshold for mappability, or low quality bases, may be replaced by the corresponding bases as found in the reference sequence. After data filtering and mapping, variant bases found between the sequence reads obtained from the subject and the reference sequence are analyzed.

In some embodiments, the mutations may be rare mutations. In some embodiments, the mutations may be low-frequency mutations. In some embodiments, a rare mutation has a minor allele frequency of <1% in the general population (also referred to as 0.01). In some mutations, a low-frequency mutation has a minor allele frequency of 1-5% (also referred to as 0.01 to 0.05). In some mutations, a common mutation has a minor allele frequency of >5% (also referred to as >0.05). In some embodiments, mutation analysis refers to analysis of rare mutations, low-frequency mutations, and/or common mutations.

For an exemplary genome, the next step comprises determining read coverage for each mappable base position, performed using either reads with barcodes, or reads without barcodes. In embodiments without barcodes, the previous mapping steps will provide coverage of different base positions. Sequence reads that have sufficient mapping and quality scores may be counted. The number of coverage reads may be assigned a score per each mappable position. In embodiments involving barcodes, all sequences with the same barcode may be collapsed into one consensus read being derived from the sample parent molecule. The sequence for each base is aligned as the most dominant nucleotide read for that specific location. Further, the number of unique molecules can be counted at each position to derive simultaneous quantification for each position. This step reduces biases which may have been introduced such as during steps involving amplification. Only reads with unique barcodes may be counted for each mappable position and influence the assigned score. Once read coverage may be ascertained and variant bases relative to the control sequence in each read are identified, the frequency of variant bases may be calculated as the number of reads containing the variant divided by the total number of reads. This may be expressed as a ratio for each mappable position in the genome. For each base position, the frequencies of all four nucleotides, i.e. cytosine, guanine, thymine, adenine, are analyzed in comparison to the reference sequence. A stochastic or statistical modeling algorithm is applied to convert the normalized ratios for each mappable position to reflect frequency states for each base variant. In some cases, this algorithm may comprise one or more of the following: Hidden Markov Model, dynamic programming, support vector machine, Bayesian or probabilistic modeling, trellis decoding, Viterbi decoding, expectation maximization, Kalman filtering methodologies, and neural networks. The discrete rare mutation states of each base position can be utilized to identify a base variant with high frequency of variance as compared to the baseline of the reference sequence. In some embodiments, the baseline might represent a frequency of at least 0.0001%, 0.001%, 0.01%, 0.1%, 1.0%, 2.0%, 3.0%, 4.0% 5.0%, 10%, or 25%. In other embodiments, the baseline might represent a frequency of at least 0.0001%, 0.001%, 0.01%, 0.1%, 1.0%, 2.0%, 3.0%, 4.0% 5.0%, 10%, or 25%. In some embodiments, all adjacent base positions with the base variant or mutation can be merged into a segment to report the presence or absence of a rare mutation. In certain embodiments, various positions can be filtered before they are merged with other segments.

After calculation of frequencies of variance for each base position, the variant with largest deviation for a specific position in the sequence derived from the subject as compared to the reference sequence is identified as a rare mutation. In some embodiments, a rare mutation may be correlated with a disease state. In certain embodiments, a rare mutation may be correlated with ROP.

A rare mutation or variant may comprise a genetic aberration that includes, but is not limited to a single base substitution, or small indels, transversions, translocations, inversion, deletions, truncations or gene truncations. In some embodiments, a rare mutation may be at most 1, 2, 3, 4, 5, 6, 7, 8, 9, 10, 15 or 20 nucleotides in length. On other embodiments, a rare mutation may be at least 1, 2, 3, 4, 5, 6, 7, 8, 9, 10, 15 or 20 nucleotides in length.

The presence or absence of a mutation may be reflected in graphical form, indicating various positions in the genome and a corresponding increase or decrease or maintenance of a frequency of mutation at each respective position. Additionally, rare mutations may be used to report a percentage score indicating how much disease material exists in the polynucleotide sample. A confidence score may accompany each detected mutation, given known statistics of typical variances at reported positions in non-disease reference sequences. Mutations may also be ranked in order of abundance in the subject or ranked by clinically actionable importance.

### Computer Systems

Methods of the present disclosure can be implemented using, or with the aid of, computer systems. For example, the methods of the present disclosure can be implement using a computer system programmed or otherwise configured to implement the methods of the present disclosure, including regulating various aspects of sample preparation, sequencing, and/or analysis. In some embodiments, the computer system is configured to perform sample preparation and sample analysis, including nucleic acid sequencing.

In some embodiments, the computer system includes a central processing unit (CPU; “processor” or “computer processor”), which can be a single core or multicore processor, or a plurality of processors for parallel processing. The computer system also includes memory or memory location (e.g., random-access memory (RAM), read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus, such as a motherboard. The storage unit can be a data storage unit or data repository for storing data. In some embodiments, the computer system can be operatively coupled to a computer network (“network”) with the aid of a communication interface. The network can be the Internet, an internet and/or extranet, or an intranet and/or extranet that is in communication with the Internet. In certain embodiments, the network is a telecommunication and/or data network. In some embodiments, the network can include one or more computer servers, which can enable distributed computing, such as cloud computing. In some other embodiments, the network, cases with the aid of the computer system, can implement a peer-to-peer network, which may enable devices coupled to the computer system to behave as a client or a server. In some embodiments, the CPU can execute a sequence of machine-readable instructions, which can be embodied in a program or software. In certain embodiments, the instructions may be stored in a memory location. Examples of operations performed by the CPU can include fetch, decode, execute, and writeback. The storage unit can store files, such as drivers, libraries and saved programs. The storage unit can store programs generated by users and recorded sessions, as well as output(s) associated with the programs. The storage unit can store user data, e.g., user preferences and user programs. In some embodiments, the computer system can include one or more additional data storage units that are external to the computer system, such as located on a remote server that is in communication with the computer system through an intranet or the Internet. In some embodiments, the computer system can communicate with one or more remote computer systems through the network. In certain embodiments, the computer system can communicate with a remote computer system of a user (e.g., operator). The operator can access the computer system via the network.

Methods as described herein can be implemented using CPU executable code stored on an electronic storage location of the computer system, for example, on the memory or electronic storage unit. In some embodiments, the machine executable or machine readable code can be provided in the form of software. During use, the code can be executed by the processor. In some embodiments, the code can be retrieved from the storage unit and stored on the memory for ready access by the processor. In some embodiments, the electronic storage unit can be precluded, and machine-executable instructions are stored on memory. The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods described herein, for example, the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors, or associated modules thereof, such as various semiconductor memories, tape drives, and disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, or optical links, may also be considered as media bearing the software. As used herein, unless restricted to non-transitory, tangible “storage” media, terms such as computer or machine “readable medium” refer to any medium that participates in providing instructions to a processor for execution. Hence, a machine readable medium, such as computer-executable code, may take many forms, including but not limited to, a tangible storage medium, a carrier-wave medium or physical transmission medium. Non-volatile storage media include, for example, optical or magnetic disks, such as any of the storage devices in any computer(s), such as may be used to implement databases. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications.

In some embodiments, the computer system can include or be in communication with an electronic display that comprises a user interface (UI) for providing, for example, one or more results of sample analysis. Examples of UI's include, without limitation, a graphical user interface (GUI) and web-based user interface.

### Methods of Treatment

The present disclosure also relates to methods of treating a subject having or suspected of having a vascular retinopathy. In some embodiments, this disclosure relates to methods of treating a subject, e.g., patient having ROP.

As used herein, a modulator is a therapeutic agent that alters the expression, level, and/or activity of a gene or gene product (including protein or RNA, e.g., mRNA, miRNA, or piRNA). In some embodiments, a modulator is administered to a subject having a gene variant, to alter a gene or gene product to similar levels as unaffected subjects who do not have the variant, or to provide similar levels of a gene or gene product as subjects carrying protective gene variants against developing a vascular retinopathy. In some embodiments, a modulator is administered to a subject to enhance the expression, level, and/or activity of a gene or gene product in subjects. In some embodiments, a modulator is administered to a subject to restore the expression, level and/or activity of a gene or gene product to baseline or to substantially the same as in a subject carrying protective gene variants, for example to expression, level and/or activity of a non-carrier. In some embodiments, a modulator is administered to a subject to reduce the expression, level and/or activity of a gene or gene product in subjects. In some embodiments, the modulator alters gene expression, e.g. by affecting a transcriptional regulator (inhibitor or activator) or inhibitory RNA. In some embodiments, a modulator alters the gene or gene product levels, e.g. by affecting the RNA product of a gene and affecting its stability or translation. In some embodiments, a modulator alters the activity of the gene or gene product, e.g. by affecting the protein product directly, activating or inhibiting the protein, or enhancing or preventing its multimerization or binding capacity, which can be through competitive, noncompetitive, or uncompetitive interactions.

In some embodiments, a modulator is an antagonist. As used herein, an antagonist is a therapeutic agent that interferes with or inhibits the physiological action, e.g., expression, level and/or activity of a target, or that inhibits or interferes with the physiological action of a positive-regulator gene or gene product (that increases the expression, level and/or activity of the target gene or gene product or decreases the expression, level and/or activity of an activator of the target). In some aspects, the antagonist can inhibit the gene or gene product directly, or activate an inhibitor of the gene or gene product, or inhibit an activator of the gene to reduce the expression, level, and/or activity of the target gene or gene product. In some embodiments, the antagonist alters expression of the gene, e.g. by inhibiting an activator of the target, or enhancing or stabilizing an inhibitor of the target. In some embodiments, the antagonist alters the gene or gene product levels by destabilizing the RNA of the gene or inhibiting its translation. In some embodiments, the antagonist alters the activity of the gene or gene product, for example by directly inhibiting the protein's activity, binding, or multimerization, or by competitively, non-competitively, or uncompetitively binding the protein to decrease its activity. As used herein, the term “antagonist” also refers to enzyme inhibitors, for example, antagonists to RAPGEF3, ADCY7, ADCY9, PRKAR1A and ADCY4. Antagonists of RAPGEF3, ADCY7, ADCY9, PRKAR1A and ADCY4 include and are not limited to: ESI-09, CE3F4, HJC0197, (Rp)-cAMPS, 7-Hydroxystaurosporine, Balanol, Chelerythrine, BX-795, BX-912, and GEM231. Additional antagonists include Beta-1, Beta-2, and Beta-3 adrenergic receptor antagonists, for example: beta-blockers, acebutolol, atenolol, betaxolol, bisoprolol, esmolol, metoprolol, nebivolol, vortioxetine, butoxamine, ICI-188,551, propranolol, L-748,328, L-748,337. In some embodiments, the antagonist is an inhibitor of all beta adrenergic receptors, for example: amiodarone, olanzapine, paroxetine, cryptenamine, metoprolol. In certain embodiments, the antagonist is an inhibitor of beta-1 adrenergic receptor, for example: lamotrigine. In some embodiments, the modulator is a general beta adrenergic receptor antagonist, for example: nortripyline, bethanidine, and bufuralol. In some embodiments, the antagonist targets beta-1 adrenergic receptor, for example: esmolol, beaxolol, metoprolol, atenolol, timolol, propranolol, bisoprolol, alprenolol, nadolol, levobunolol, metipranolol, labetalol, carvedilol, bevantolol, practolol, oxprenolol, dronedarone, nebivolol, sotalol, bupranolol, celiprolol, asenapine, terbutaline, propafenone, levobetaxolol, artinolol, ranolazine, iloperidone, carteolol, penbutolol, and bopindolol. In some embodiments, the antagonist targets beta-2 adrenergic receptor, for example: carteolol, timolol, propranolol, labetalol, bisoprolol, alprenolol, desipramine, nadolol, levobunolol, metipranolol, carvedilol, oxyprenolol, metoprolol, betaxolol, sotalol, bevantolol, bupranolol, nebivolol, asenapine, atenolol, propafenone, arotinolol, viloxazine, iloperidone, penbutolol, and bopindolol. In some embodiments, the antagonist targets beta-3 adrenergic receptor, for example: propranolol, bupranolol, and alprenolol.

In some embodiments, a modulator is an agonist. As used herein, an agonist is a therapeutic agent that activates or enhances the physiological action, e.g., expression, level and/or activity of a target, or that activates a positive regulator of the target (a gene or gene product that increases the expression, level and/or activity of a target or that interferes with or inhibits a negative regulator of the target). In some aspects, the agonist can activate the gene or gene product directly, or inhibit an inhibitor of the gene or gene product, or activate or upregulate an activator of the gene to increase the expression, level, and/or activity of the target gene or gene product. In some embodiments, the agonist alters expression of the gene, e.g. by enhancing an activator of the target, or inhibiting or destabilizing an inhibitor of the target. In some embodiments, the agonist alters the gene or gene product levels by stabilizing the RNA of the gene or enhancing its translation. In some embodiments, the agonist alters the activity of the gene or gene product, for example by directly activating the protein's activity, binding, or multimerization, or by competitively, non-competitvely, or uncompetitively binding the protein to increase its activity. As used herein, the term “agonist” also refers to enzyme activators, for example, agonists to RAPGEF3, ADCY7, ADCY9, PRKAR1A and ADCY4. Agonists of RAPGEF3, ADCY7, ADCY9, PRKAR1A and ADCY4 include and are not limited to: 8-CPT-2Me-cAMP, 8-pCPT-2-O-Me-cAMP-AM, Cyclic adenosine monophosphate, and N6-benzyl-cAMP. Additional agonists include Beta-1, Beta-2, and Beta-3 adrenergic receptor agonists, for example: isoprenaline, denopamine, dobutamine, xamoterol, isoproterenol, bitolterol, fenoterol, hexoprenaline, isoprenoline, levosalbutamol, orciprenaline, pirbuterol, procaterol, salbutamol, terbutaline, arformoterol, bambuterol, clenbuterol, formoterol, salmeterol, carmoterol, indacaterol, milveterol (GSK 159797), olodaterol, vilanterol (GSK 642444), isoxsuprine, ritodrine, albuterol, terbutaline, zilpaterol, amibegron (SR-58611A), BRL-37344, CL-316,243, L-742,791, L-796,568, LY-368,842, mirabegron (YM-178), nebivolol, Ro40-2148, solabegron (GW-427,353), and vibegron (MK-4618). In some embodiments, the modulator is a general beta adrenergic receptor agonist, for example: amphetamine, mephentermine, DL-methylephedrine, and ritodrine. In some embodiments, the agonist targets beta-1 adrenergic receptor, for example: isoetharine, dobutamine, isoprenaline, arbutamine, epinephrine, norepinephrine, droxidopa, albuterol, pirbuterol, fenoterol, phylpropanolamine, clenbuterol, ephedra sinica root, formoterol, racepinephrine, DL-methylephedrine, ephedrine, mirabegron, and pseudoephedrine. In some embodiments, the agonist targets beta-2 adrenergic receptor, for example: orciprenaline, ritodrine, terbutaline, salmeterol, formoterol, albuterol, ephinephrine, arformoterol, procaterol, clenbuterol, fenoterol, pirbuterol, norephinephrine, bambuterol, indacaterol, droxidopa, arbutamine, dobutamine, dipivfrin, ephedra sinica root, isoetharine, phenylpropanolamine, oladaterol, vilanterol, celiprolol, levosalbutamol, doxofylline, protokylol, racepinephrine, etafedrine, bedradrine, ephedrine, and isoprenaline. In some embodiments, the agonist targets beta-3 adrenergic receptor, for example: norepinephrine, droxidopa, solabegron, fenoterol, clenbuterol, arbutamine, isoprenaline, ephedra sinica root, mirabegron, formoterol, terbutaline, pindolol, recapinephrine, celiprolol, nebivolol, vibegron, and dihydroergotamine.

In some embodiments, the modulator is a gene knockdown agent that is siRNA, shRNA, antisense RNA, or a gene knockout agent that is a transcription activator-like effector nuclease (TALEN) or a zinc finger nuclease (ZFN). In some aspects, the modulator is a gene activating agent that is siRNA or shRNA.

In certain embodiments, the method of treating a subject having or suspected of having a vascular retinopathy comprises:
a. obtaining or having obtained a sample from the subject;
b. detecting or having detected one or a plurality of gene variants of RAPGEF3: rs2240079, rs11168215, rs11168214 and rs61917617; ADCY7: rs55683248; ADCY9: rs2072341; PRKAR1A: rs8082254 and rs72847785; and ADCY4: rs3181252 and rs17256902 in the sample from the subject; and
c. administering a therapeutically effective amount of a modulator of RAPGEF3, ADCY7, or ADCY9 to the subject having one or a plurality of gene variants of RAPGEF3: rs2240079, rs11168215, rs11168214 and rs61917617; ADCY7: rs55683248; or ADCY9: rs2072341;
or administering a therapeutically effective amount of a modulator of PRKAR1A or ADCY4 to the subject having one or a plurality of gene variants of RAPGEF3: rs2240079, rs11168215, rs11168214 and rs61917617; ADCY7: rs55683248; or ADCY9: rs2072341;
or administering a therapeutically effective amount of a modulator of PRKAR1A or ADCY4 to the subject having one protective or no gene variant from the group consisting of: rs8082254 rs72847785, rs3181252 and rs17256902.

In certain embodiments, the method of treating a subject, e.g., patient having ROP comprises:
a. determining or having determined if the subject, e.g., patient has a mutation in RAPGEF3, ADCY7, ADCY9, PRKAR1A or ADCY4; and
b. if the subject, e.g., patient has a mutation in RAPGEF3, ADCY7, or ADCY9, further determining whether the patient has one or a plurality of gene variants of RAPGEF3: rs2240079, rs11168215, rs11168214 and rs61917617; ADCY7: rs55683248; ADCY9: rs2072341 then administering a therapeutically effective amount of a modulator of RAPGEF3, ADCY7, or ADCY9; or
c. if the subject, e.g., patient has a mutation in PRKAR1A or ADCY4, further determining whether the patient has one or a plurality of gene variants of PRKAR1A: rs8082254 and rs72847785 and ADCY4: rs3181252 and rs17256902 then administering a therapeutically effective amount of a modulator of PRKAR1A or ADCY4 if the subject, e.g., patient carries one or no protective gene variant.

In certain embodiments, this disclosure provides for a method of slowing vision loss or improving the vision in a subject having or suspected of having a vasulcar retinopathy, the method comprising administering to the subject a therapeutically effective amount of a modulator of RAPGEF3, ADCY7, ADCY9, PRKAR1A or ADCY4.

In some embodiments, the method of treating a subject having or suspected of having a vascular retinopathy, the method of treating a subject having ROP, or the method of slowing vision loss or improving the vision in a subject having or suspected of having a vascular retinopathy further comprises administering one or more additional active agents or supportive therapies for treating, preventing, or reducing the severity of an eye disorder to the subject. In certain embodiments, the one or more supportive therapies is selected from the group consisting of: surgery, laser therapy, photocoagulation, anti-angiogenic therapy, vitrectomy, scleral buckle surgery, and pneumatic retinopexy, or any combination thereof. In other embodiments, the one or more additional active agents is selected from the group consisting of: VEGF inhibitors, placental growth factor (PIGF) inhibitor, bevacizumab, ranibizumab, aflibercept, Ca²⁺ inhibitors, flunarizine, nifedipine, cryotherapy, hyperbaric oxygenation, Na⁺ channel blockers, topiramate, iGluR antagonists, (MK-801, dextromethorphan, eliprodil, flupirtine, antioxidants, dimethylthiourea, alpha-lipoic acid, superoxide dismutase, catalase, desferrioxamine, mannitol, allopurinol, calcium dobesilate, trimetazidine, EGB-761, anti-inflammatory agents, cyclodiathermy, cyclocryotherapy, ocular filtering procedures, implantation of drainage valves, antiplatelet therapy, aspirin, ticlopidine, clopidogrel, anticoagulant therapy, warfarin, heparin, steroids, systemic or local corticosteroids, prednisone triamcinolone, fluocinolone acetonide, dexamethasonc, steroid-sparing immunosuppressants, cyclosporine, azathioprine, cyclophosphamide, mycophenolate, mofetil, infliximab, etanercept, dietary supplements, vitamin C, vitamin E, lutein, zinc, folic acid, vitamin B6, vitamin B12, zeaxanthin, a VEGF and PIGF inhibitor, or any combination thereof.

In certain embodiments, the modulator comprises a polypeptide capable of binding or sequestering RAPGEF3, ADCY7, ADCY9, ADCY4, or PRKAR1A. In some aspects, the polypeptide capable of binding or sequestering RAPGEF3, ADCY7, ADCY9, ADCY4, or PRKAR1A comprises an antibody, antibody fragment, ScFv, Fv, Fd, Fab, Fab′, F(ab)′2, VH domain, VL domain, monoclonal antibody, polyclonal antibody, Fc or fusion protein or any combination thereof to said RAPGEF3, ADCY7, ADCY9, ADCY4, or PRKAR1A, or an epitopic portion thereof.

As used herein, the term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions are further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. An immunoglobulin may derive from any of the commonly known isotypes, including but not limited to IgA, secretory IgA, IgG and IgM. IgG subclasses also include but are not limited to human IgG1, IgG2, IgG3 and IgG4. “Isotype” refers to the Ab class or subclass (e.g., IgM or IgG1) that is encoded by the heavy chain constant region genes. The term “antibody” can include or exclude both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. The term “antibody” also includes an antigen-binding fragment or an antigen-binding portion of any of the aforementioned immunoglobulins, and includes a monovalent and a divalent fragment or portion, and a single chain Ab.

### Methods of Screening

The present disclosure also relates to methods of screening for a gene knockout or gene knockdown treatment and screening for a gene knock-in or gene activation treatment. In some embodiments, the method of screening for a gene knockout or gene knockdown treatment, or a gene knock-in or gene activation treatment, the method comprising performing PCR for gene variants RAPGEF3: rs2240079, rs11168215, rs11168214 and rs61917617; ADCY7: rs55683248; and ADCY9: rs2072341 in an animal model of oxygen-induced retinopathy: kitten, beagle puppy, rat, or mouse, then administering the gene knockdown or gene knockout treatment to the animal model, then observing the knockdown or knockout effect. In some embodiments, the method of screening for a gene knock-in or gene activation treatment, or gene knockout or gene knockdown treatment, the method comprising performing PCR for gene variants PRKAR1A: rs8082254 and rs72847785, and ADCY4: rs3181252 and rs17256902 in an animal model of oxygen-induced retinopathy: kitten, beagle puppy, rat, or mouse, then administering the gene knock-in or gene activation treatment to the animal model, then observing the knock-in or gene activation effect.

### Embodiments of the Invention

In certain embodiments, the present invention provides methods for detecting gene variants in a sample from a subject having or suspected of having a vascular retinopathy.

In certain embodiments, the present invention provides methods for detecting gene variants in a sample from a subject having ROP.

In certain embodiments, the present invention provides methods for preparing gene amplicons or enriched nucleic acid samples.

In certain embodiments, the present invention provides a method of treating a disease or disorder in a subject, comprising administering to the subject a therapeutically effective amount of a complex or a composition as described herein.

In certain embodiments, the present invention provides methods of treating a subject having or suspected of having a vascular retinopathy.

In certain embodiments, the present invention provides methods of treating a subject having ROP.

In certain embodiments, the present invention provides a method of slowing vision loss or improving the vision in a subject having or suspected of having a vascular retinopathy.

In certain embodiments, the present invention provides methods of screening for a gene knockout or gene knockdown treatment and screening for a gene knock-in or gene activation treatment.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

C. Certain Exemplary Sequences
RAPGEF3 forward primer for cDNA nucleotides
4510-5170:
(SEQ ID NO: 1)
CTTCCTTCATTTCTCCACCTG
RAPGEF3 reverse primer for cDNA nucleotides
4510-5170:
(SEQ ID NO: 2)
TCTGTGTCCTCTTGCCTGC

### EXAMPLES

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Thus, for example, in each instance herein, and in embodiments or examples of the present invention, any of the terms “comprising”, “consisting essentially of”, and “consisting of” may be replaced with either of the other two terms in the specification. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation agreed to and expressly adopted in a responsive writing by Applicants.

### Example 1: Study Design

### A. Participants

Permission to perform genetic research on human participants with ROP, or without ROP and full-term controls was granted by the Institutional Review Board at the Smith-Kettlewell Eye Research Institute. Written informed Consent for the research was obtained from the parents/guardians of the infant participants, and the study was performed in accordance with the declaration of Helsinki and the Health Insurance Portability and Accountability Act (HIPAA) regulations. The Interdisciplinary Committee on Ethics in Human Research of Memorial University also approved the research. Premature participants had a weight at birth qual or less than 1786 g and a gestational age of 34 weeks or less. Participants were painlessly sampled by rubbing the inside of the cheek with a swab optimized for DNApreservation from Norgen Biotek Corp. (Thorold, ON, Canada).

A total of 120 participants were approached and 117 consented to take part in this study with a response rate of 100%. 67 DNA samples were sequenced as the discovery cohort, including 19 affected premature infants with ROP Type 1 requiring treatment, 31 unaffected premature infants without ROP, and 17 unaffected full-term pregnancy participants. A total of 17 additional participants and as well as two repeated discovery participants were included in a validation cohort, including 12 affected premature infants with ROP Type 1 requiring treatment; 3 unaffected premature infants without ROP; and 4 unaffected full-term pregnancy participants. For the ROP Cohort, ROP was classified according to the locations (zones) and presence or absence of Type 1 ROP (Good, WV. Final result of the early treatment for retinopathy of immaturity (ETROP) randomized trial. Trans Am Opthahmol Soc. 102:233-250 (2004)). Type I ROP was defined as Zone I with plus disease (tortuosity and dilatation of retinal vessels) or stage 3 with or without plus disease, stage 2 and/or 3. No infants had less than Type I disease in this study, and thus all who had ROP required treatment. There were no infants with Zone I disease. In this cohort, all had at least 1 clock hour of stage 3 ROP (presence of neovascularization at the leading edge of a line demarcating the vascularized retina from avascular retina). One infant had stage 4A in one eye and 4B in the fellow eye. All others had favorable retinal outcomes.

Genomic DNA was extracted using the saliva DNA extraction kit (Norgen Biotek Corp.). Data from each individual was analyzed at Memorial University where statistical analysis was performed.

B. DNA sequencing

High quality DNA samples with a yield of 500 ng and above were sent to LC Sciences, LLC (Houston, TX), where targeted genome sequencing for 67 of the participants was performed for twenty components of the ADRβ signaling pathway. These components were: Three ADRB subtypes (1, 2, and 3); ten ADCY isoforms, Class I to X including the non-cardiac ADCY VIII; four subtypes of protein kinase A (PRKAR1A, PRKAR2A, PRKAR1B, PRKAR2B); RAPGEF3, the tyrosine kinase Src, and cortactin. The genome sequencing was performed through LC Sciences VariantBaits targeted sequencing services which involved a hybridization based capture methodology using specific probes for each gene (TABLE 4). The Novaseq 6000 platform (Illumina) was used with 150 base pairs (bp) paired-end mode. Binary Sequence Alignment Map (BAM) files were provided by LC Sciences after aligning the sequenced data to the hgl9 human reference genome, which were then used for subsequent analyses.

### C. Genetic Analyses

The 20 targeted genes' coordinates on hg19 reference genome plus 50 kb on flanking sides were used to extract mapped sequence data from the BAM files by a bioinformatic program bcftools (Danecek, P., et al. Twelve years of SAMtools and BCFtools. Gigascience. 10(2):giab008 (2021)). Variants in the targeted genes were calledby bcftools with multiple sample method and quality filters of minimum mapping quality of 20 and minimum base quality of 30. A variant call format (VCF) file was then generated and the variant identities (ID) were annotated to the dbsnp build 156 for hg19 reference genome (https://ftp.ncbi.nih.gov/snp/). Given the sample size of the discovery cohort, there was insufficient statistical power to examine multiallelic variants. Therefore, only bi-allelic variants including, SNVs and small insertions and deletions (indels), were extracted from the VCF file and converted to a PLINK binary format file for subsequent association tests. The allelic association test was performed for ROP in PLINK program with Fisher's exact test method (https://www.cog-genomics.org/plink/2.0/). Multiple testing was not considered as the present study focused on a hypothesis-driven targeted genetic association analysis. The significance level was defined at alpha=0.05. In addition, linkage disequilibrium analyses were performed where two or more variants associated with ROP were identified in the same gene. All other data analyses were done in STATA/SE 18.0.

### D. Validation

PCR of a genomic DNA fragment of RAPGEF3 (cDNA nucleotides 4510 to 5170) using forward and reverse primers (respectively, 5′ CTTCCTTCATTTCTCCACCTG 3′ (SEQ ID NO: 1) and 5′ TCTGTGTCCTCTTGCCTGC 3′ (SEQ ID NO: 2)) was performed on participants from the validation cohort to methodologically validate the discovery results and verify the allele frequency of three separate discovered variants, rs11168215, rs11168214 and rs61917617 associated with ROP in the discovery cohort. The PCR reactions were analyzed by agarose gel electrophoresis to confirm the amplification of a ˜661 base pair (bp) fragment. PCR reactions were then digested with either Bsu36I, BsaWI and SacII to detect the presence of respectively rs11168215, rs11168214 and rs61917617 variants. Digestions were analyzed by agarose gel electrophoresis to confirm the size of the fragments.

### Example 2: Characteristics of ROP Study Participants

The characteristics of the study participants are presented in TABLE 1. A total of 50 premature participants with or without ROP were included in the discovery cohort while the validation cohort included 14 additional premature participants and 1 repeat participant from the discovery cohort. For both cohorts, as expected, ROP participants had a lower birth weight and gestational age compared to participants without ROP (no ROP) (TABLE 1). Sex and ethnicity of the participants in the two groups of premature infants were not significantly different (p>0.05) (TABLE 1).

### Example 3: ROP Allelic Association Analysis

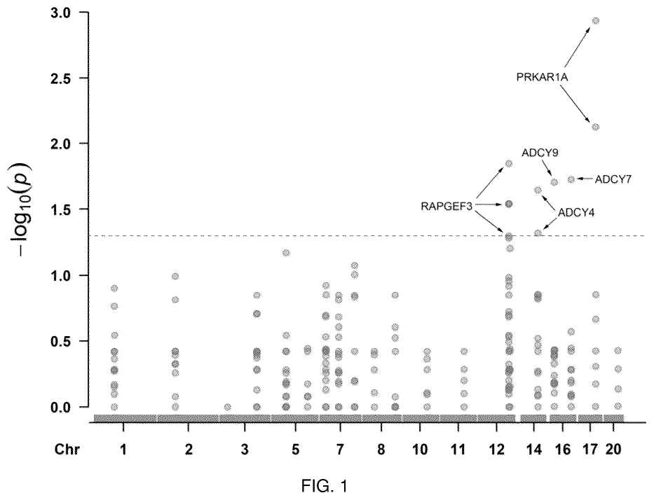

Twenty genes involved in the ADRβ signaling pathway were sequenced from the discovery cohort which included 19 premature participants with ROP Type I, 31 premature participants without any ROP and 17 full-term participants. A total of 547 bi-allelic genetic variants were identified in the sequencing data of the 67 samples including 534 SNVs and 13 indels. Four variants were removed since they were out of the Hardy-Weinberg Equilibrium with a p value <0.05. The analysis resulted in the inclusion of a total of 543 variants. Allelic association tests were performed between the ROP and no ROP groups for each of these 543 genetic variants. FIG. 1. summarizes the results of the association tests. Ten SNV located within five genes were identified to be associated with ROP with a p value <0.05 (TABLE 2). The two most significant associations to ROP were found within PRKAR1A gene (all p<0.008) as two SNVs (rs8082254 and rs72847785) located in the non-coding regions (one in the 3′UTR and one in an intron). The two rare alleles of these two PRKAR1A SNVs were found to have a protective effect on ROP (TABLE 2). Four variants in the 3′UTR of RAPGEF3 gene including rs2240079, rs11168215, rs61917617, and rs11168214 were associated with an increased risk of ROP for the rare alleles of these SNVs (TABLE 2). Additional variants associated with ROP included one SNV located in an intron of ADCY7 (rs55683248); another SNV located in an intron of ADCY9 (rs2072341); and two SNVs in ADCY4, one synonymous variant (rs3181254: no change in coding sequence) and one located in an intron (rs17256902) (TABLE 2). For ADCY7 SNV rs55683248, none of the no ROP participants carried the rare allele, whereas none of the ROP participants carried the rare allele of rs3181252 in ADCY4. ADCY7 and ADCY9 SNVs were associated with increased risk of ROP while the two ADCY4 SNVs were associated with reduced risk of ROP.

Since birth weight and gestational age at birth were also associated with ROP, we examined the association between each of the identified ROP-associated SNVs and birth weight and gestational age. However, no significant association was observed (all p>0.05). Additionally, none of the variants listed in TABLE 2 were associated with prematurity. An analysis of the variants associated with the premature groups, ROP and no ROP, versus full-term participants confirmed that there was no correlation between the variants listed in TABLE 2 and prematurity. The variants associated with prematurity will be reported elsewhere.

### Example 4: Linkage Disequilibrium Analyses

Linkage disequilibrium analyses (TABLE 3) revealed that the two SNVs identified in the non-coding regions of PRKAR1A, rs8082254 and rs72847785, correlate to each other with a coefficient of r²=0.95, indicating that they correspond to a single locus associated with ROP. Similarly, the four SNVs identified in the 3′UTR of RAPGEF3, rs2240079, rs61917617, rs11168214, and rs11168215 correlate to each other with coefficients r² ranging from 0.88 to 1, indicating that they also represent a single locus associated with ROP. Therefore, any one of these markers can be used in detecting vascular retinopathy such as ROP.

### Example 5: Validation and Combined Analyses for RAPGEF3 Variants Rs61917617, rs11168215, and rs11168214

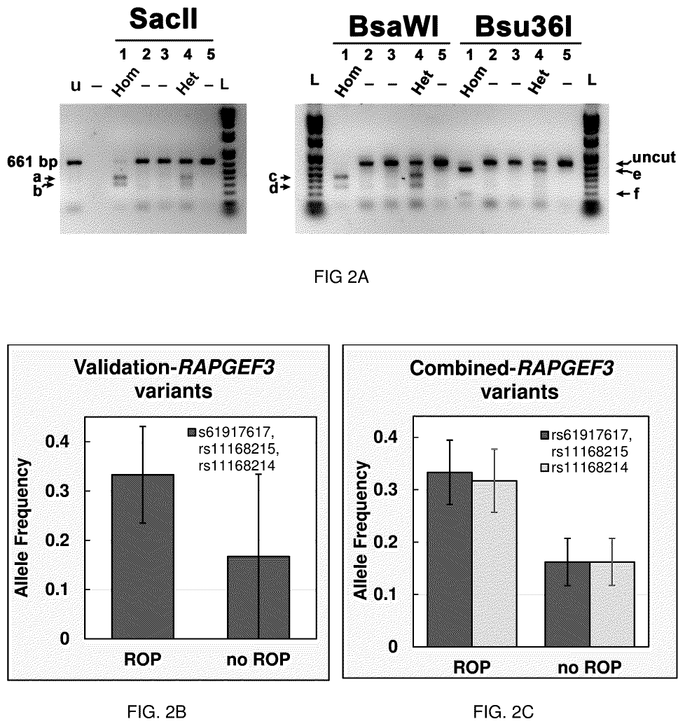

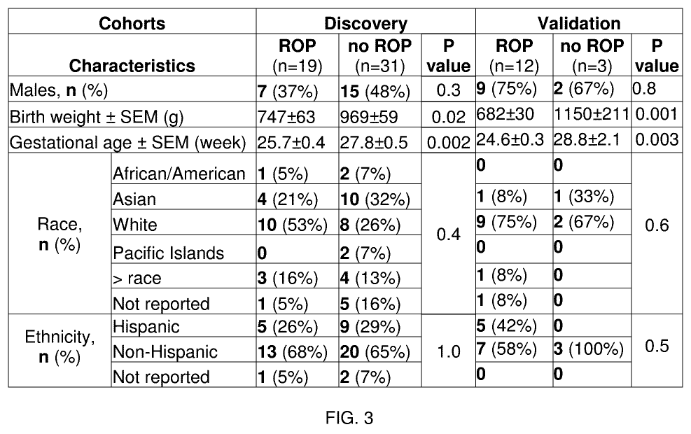

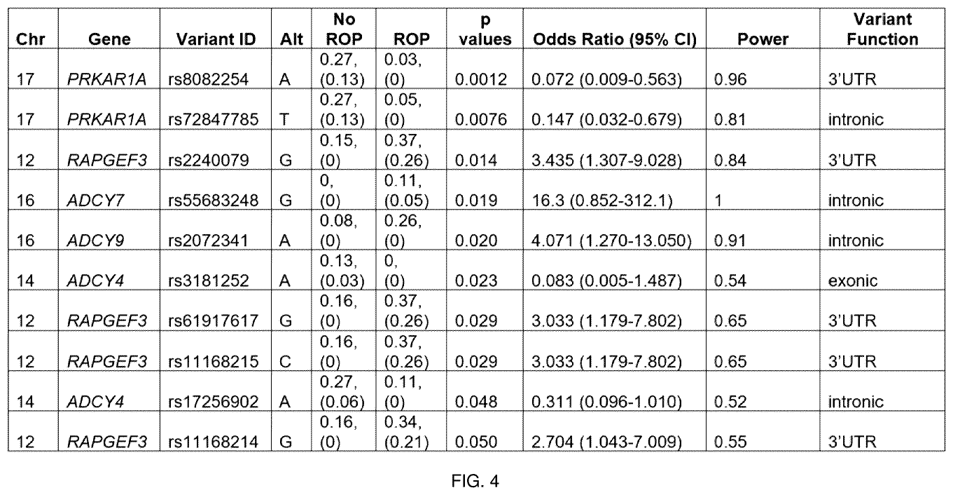

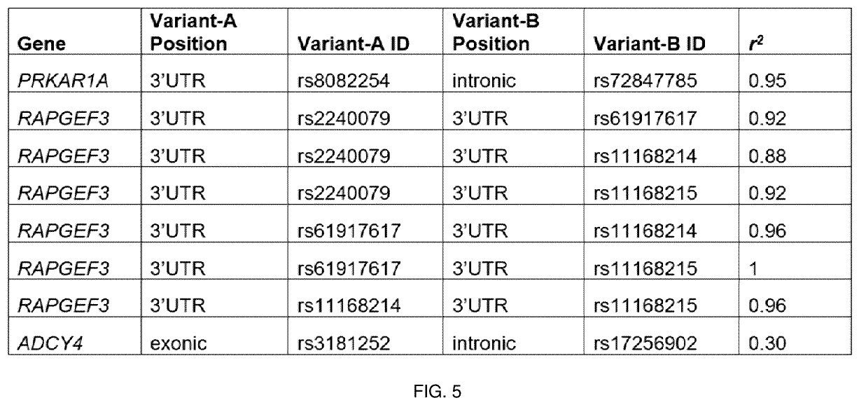

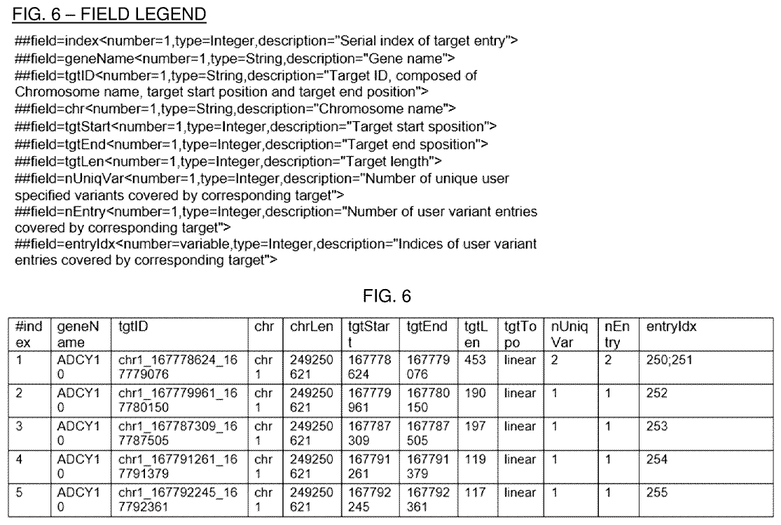

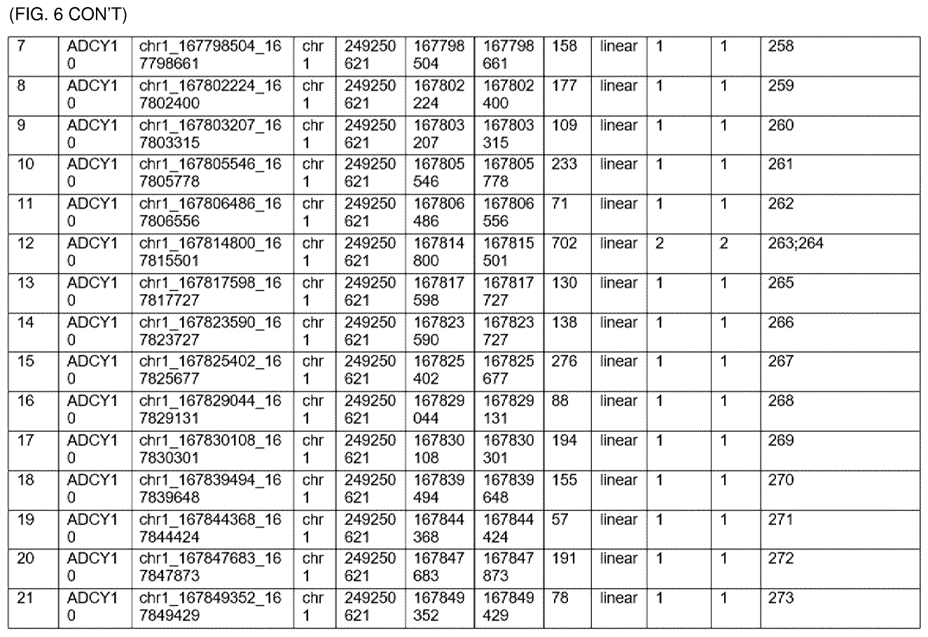

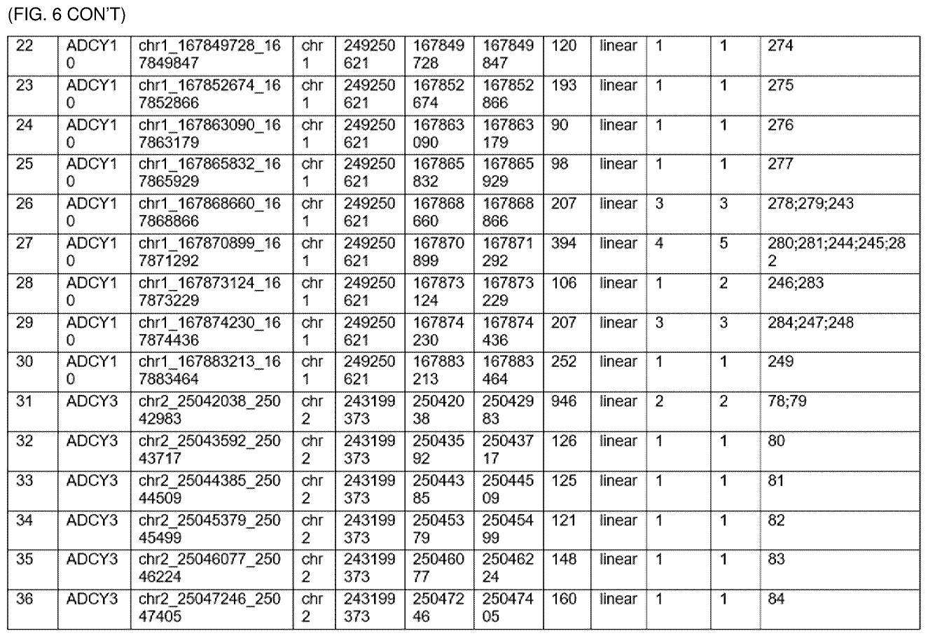

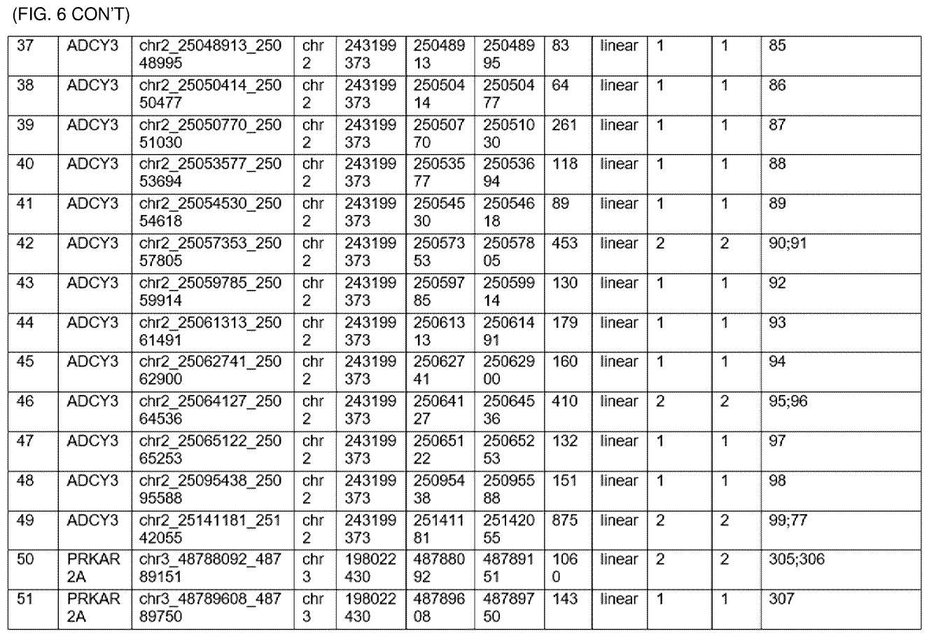

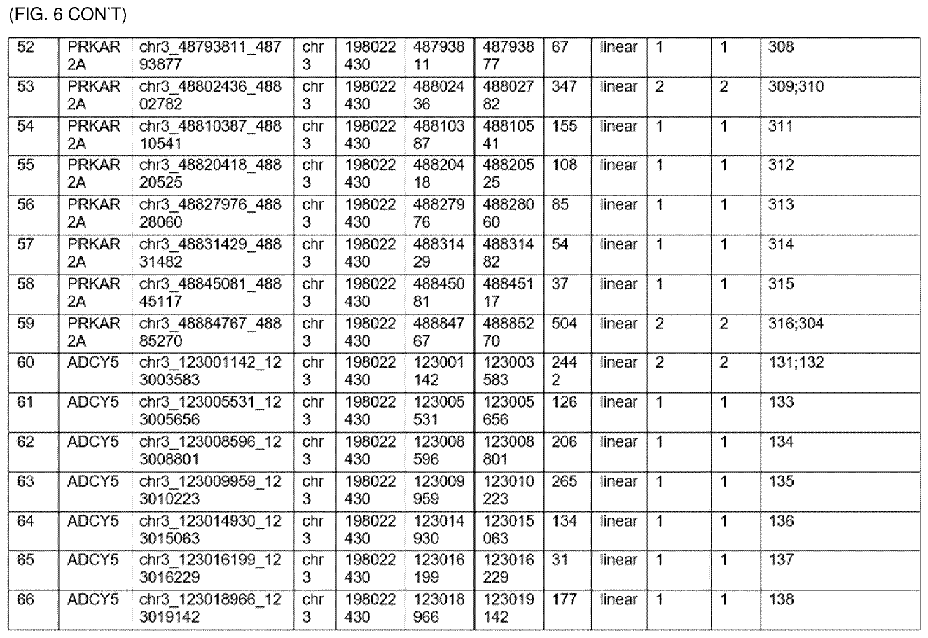

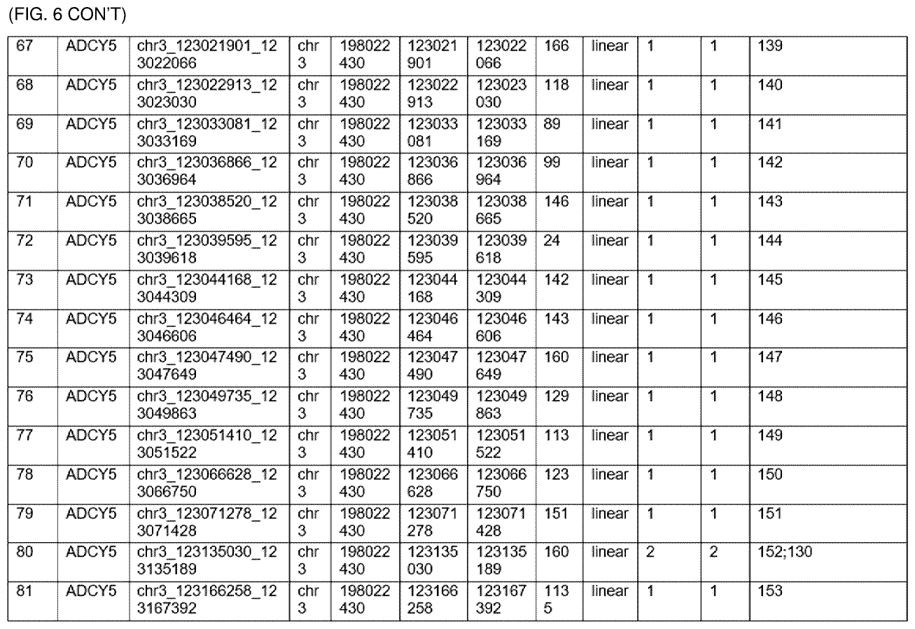

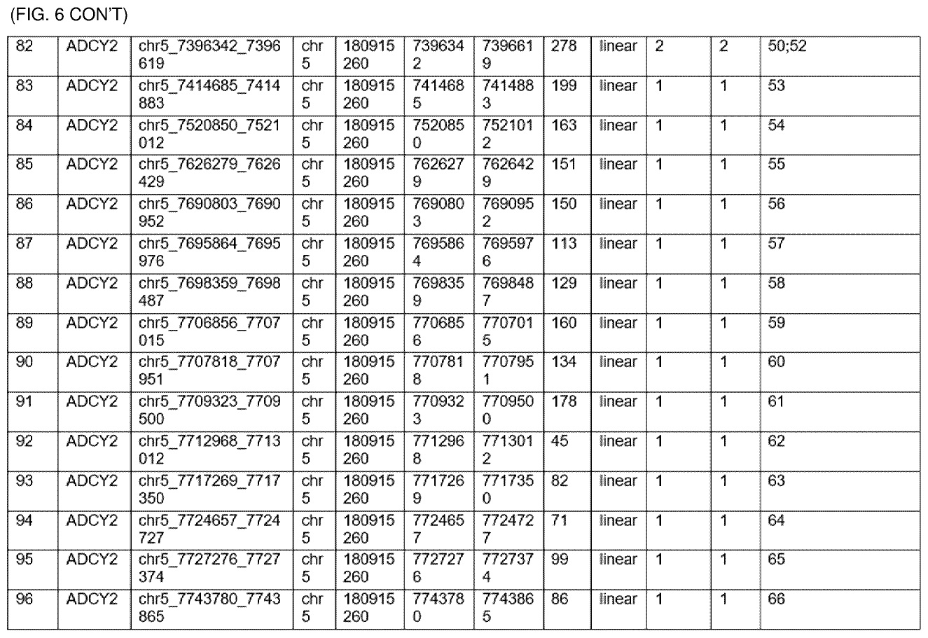

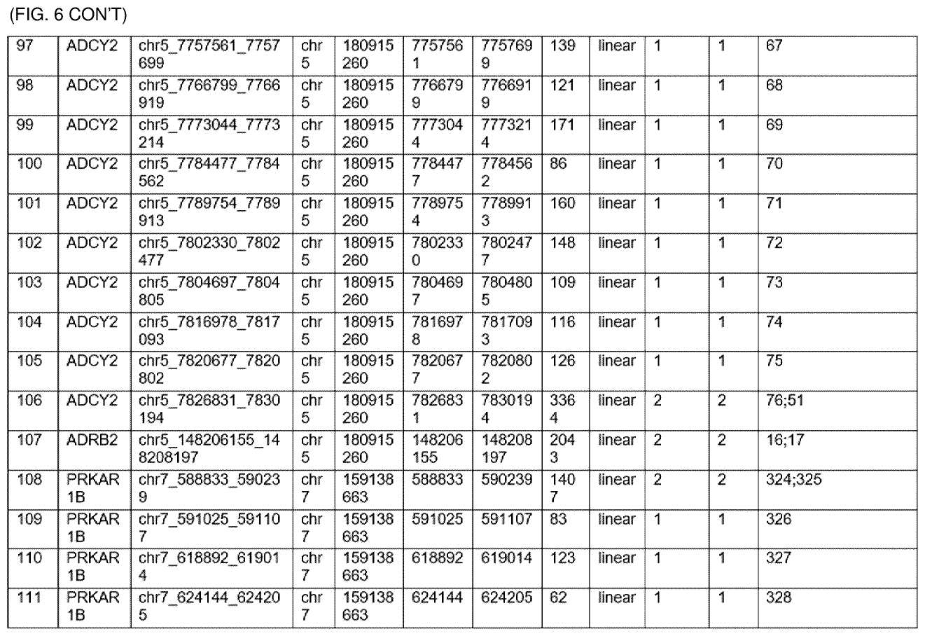

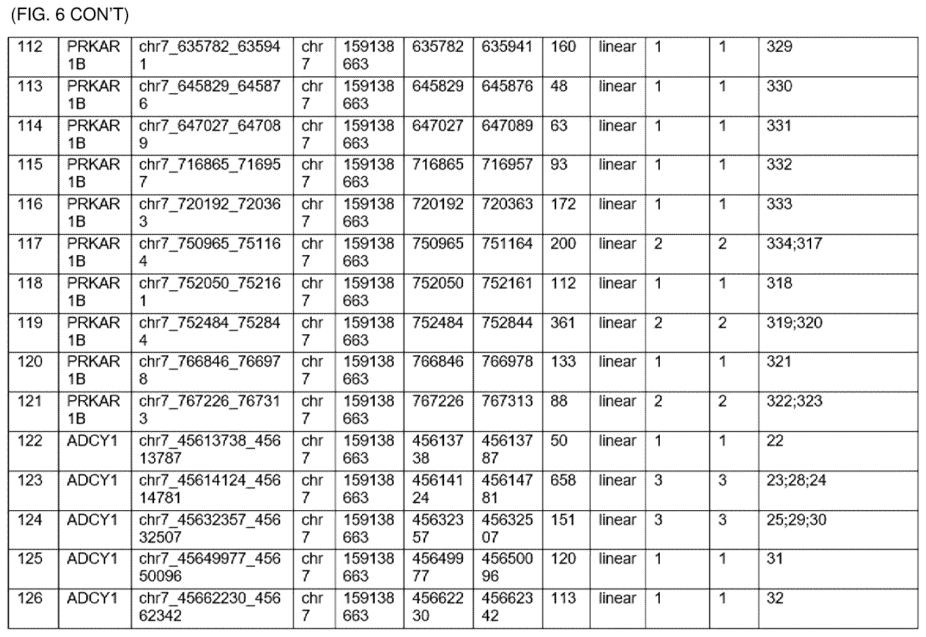

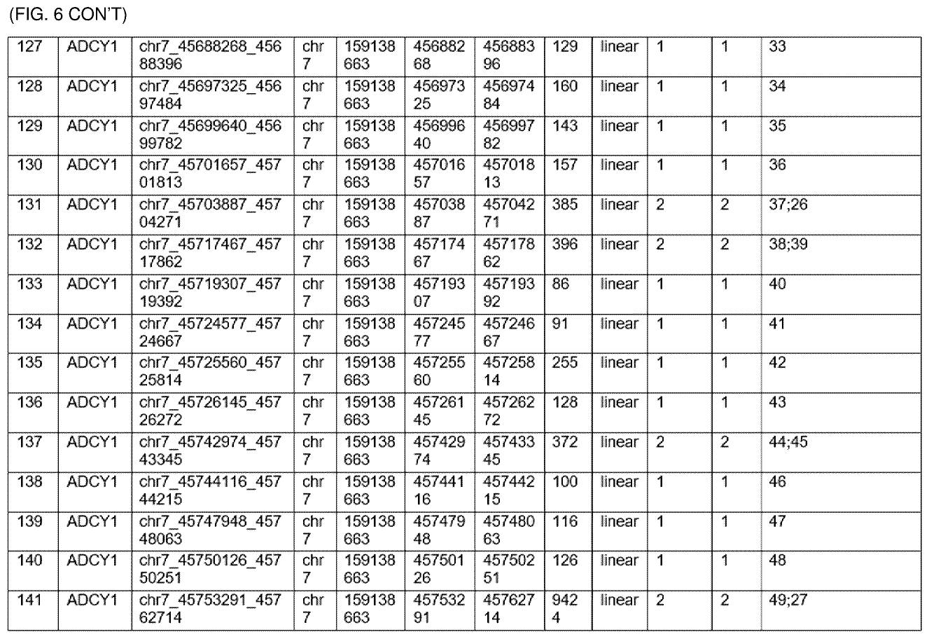

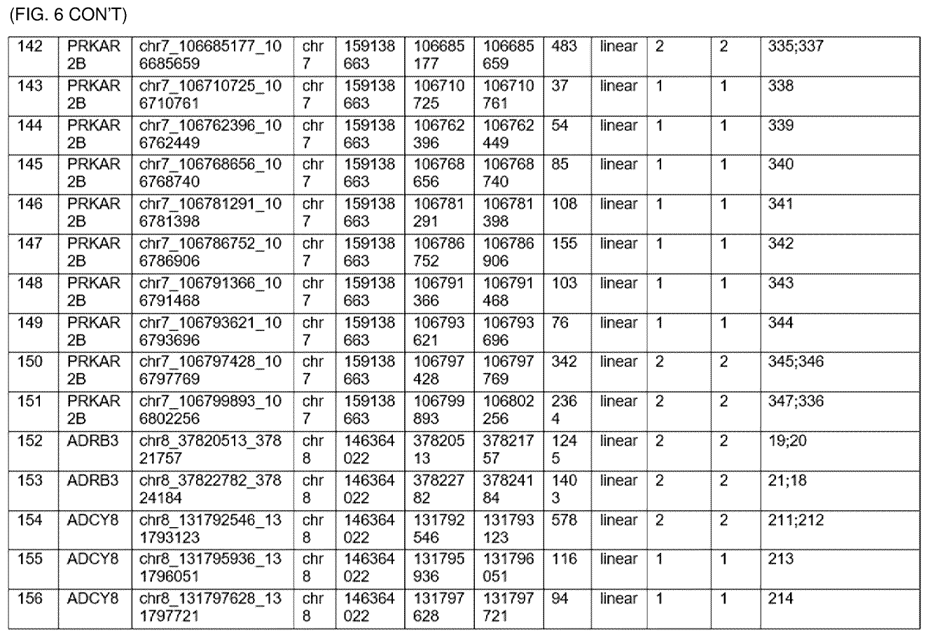

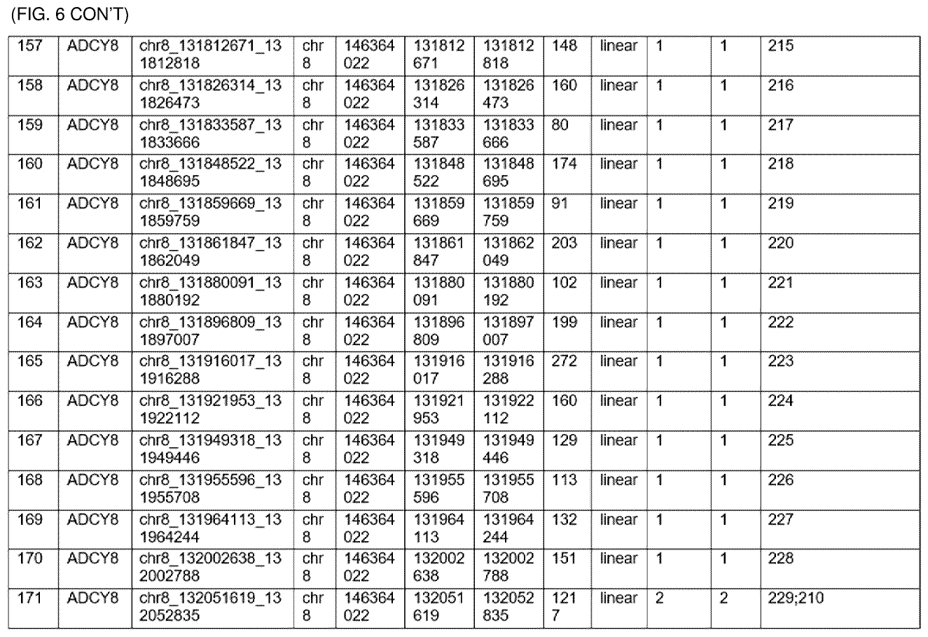

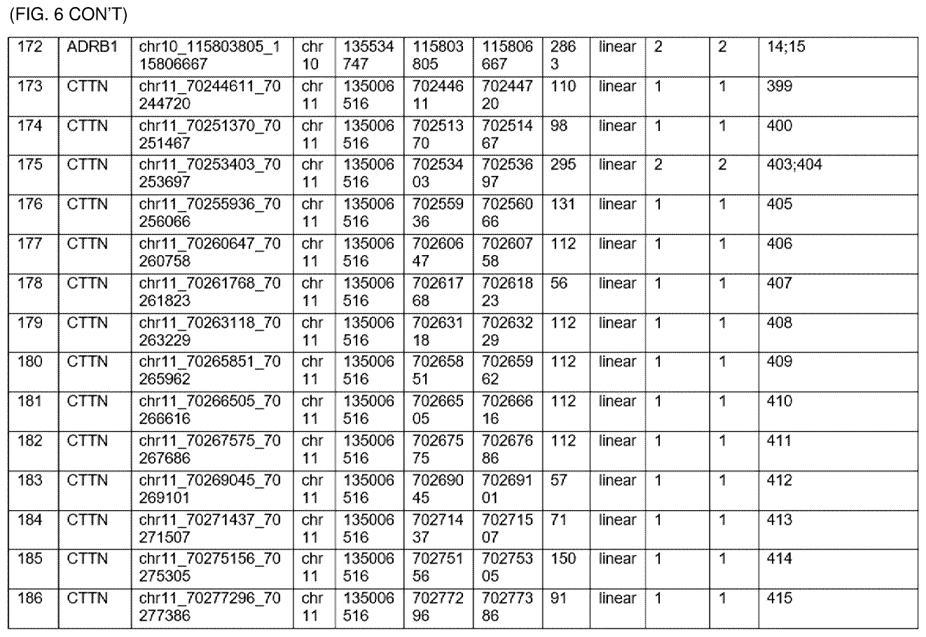

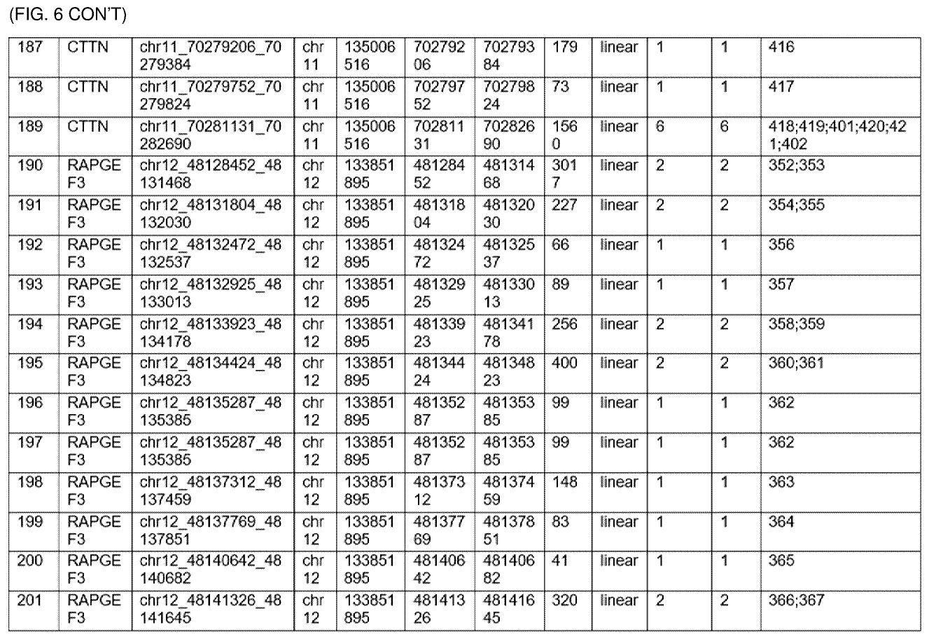

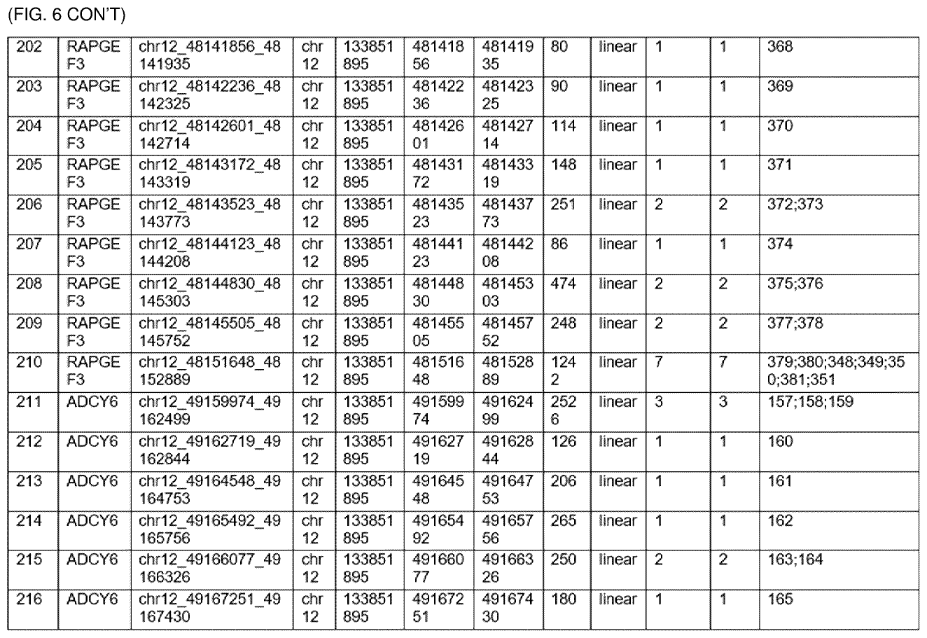

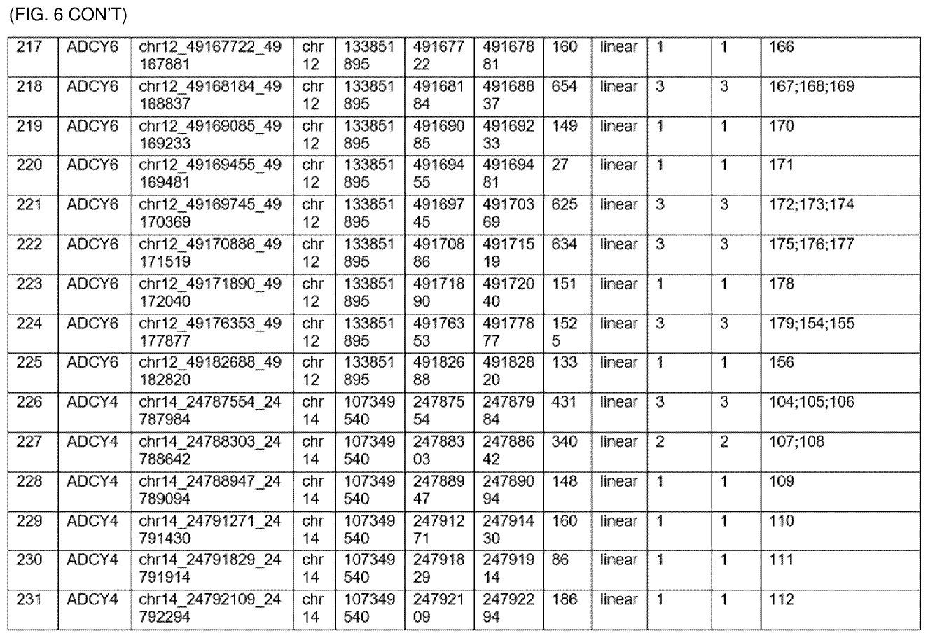

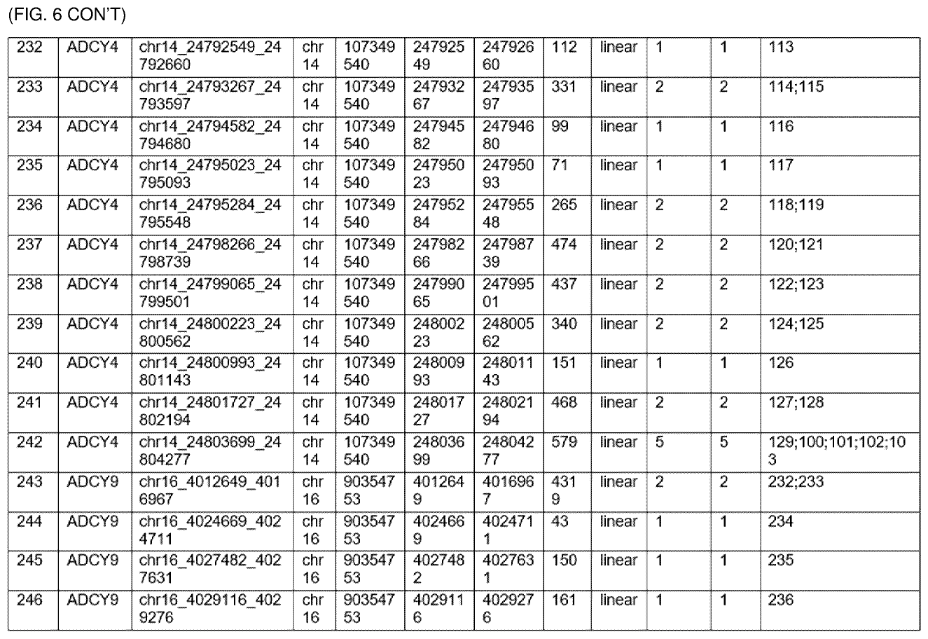

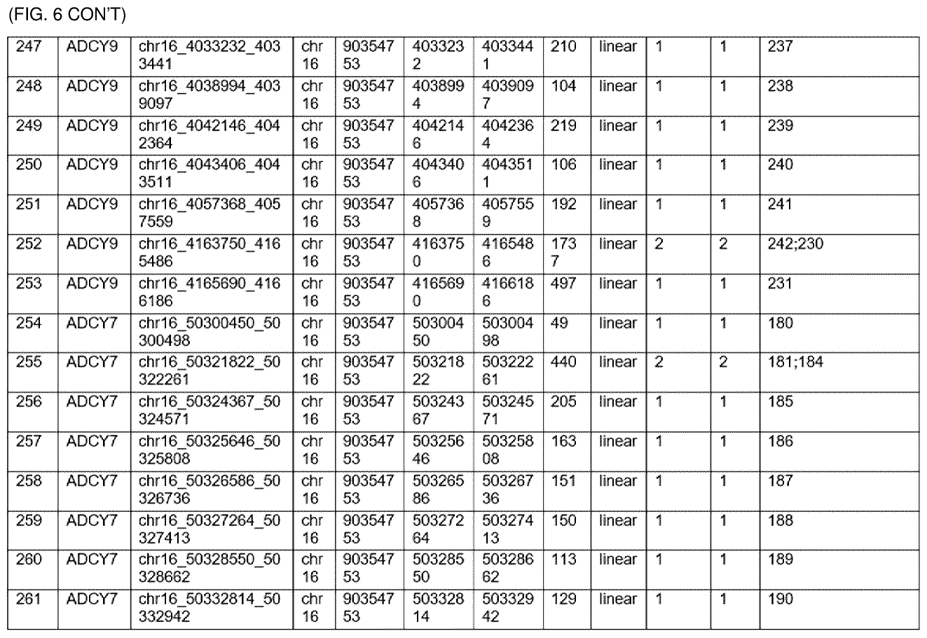

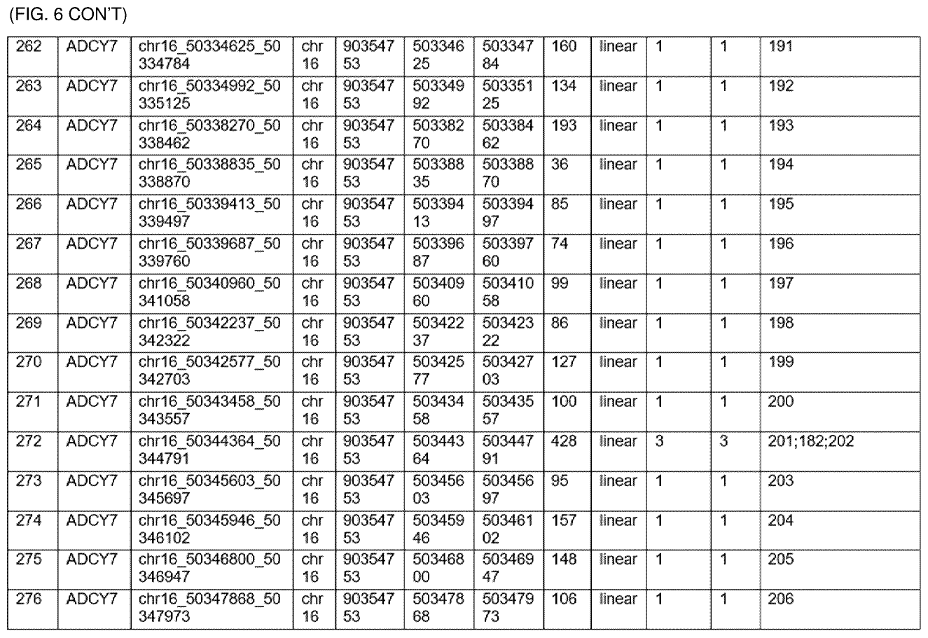

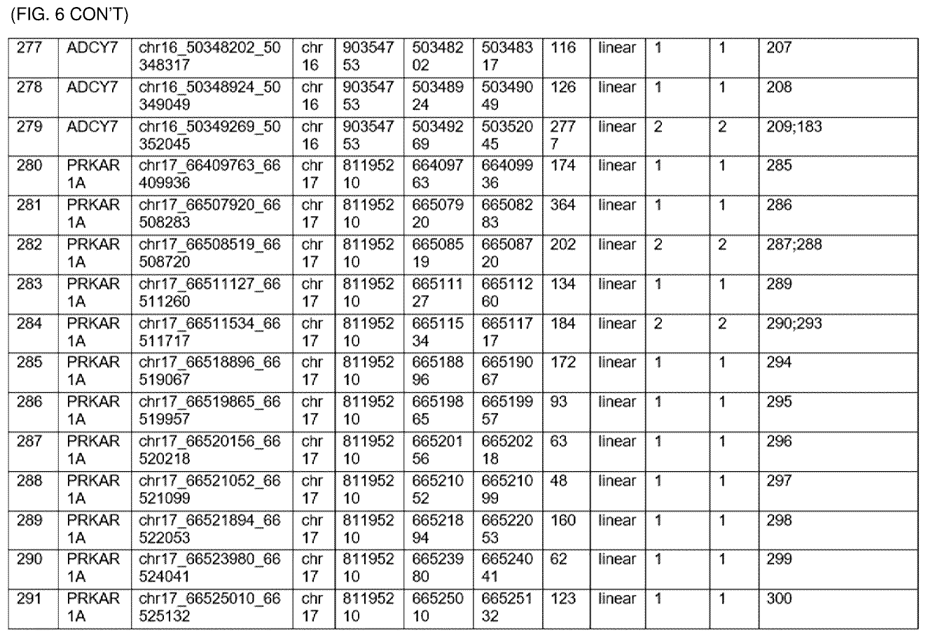

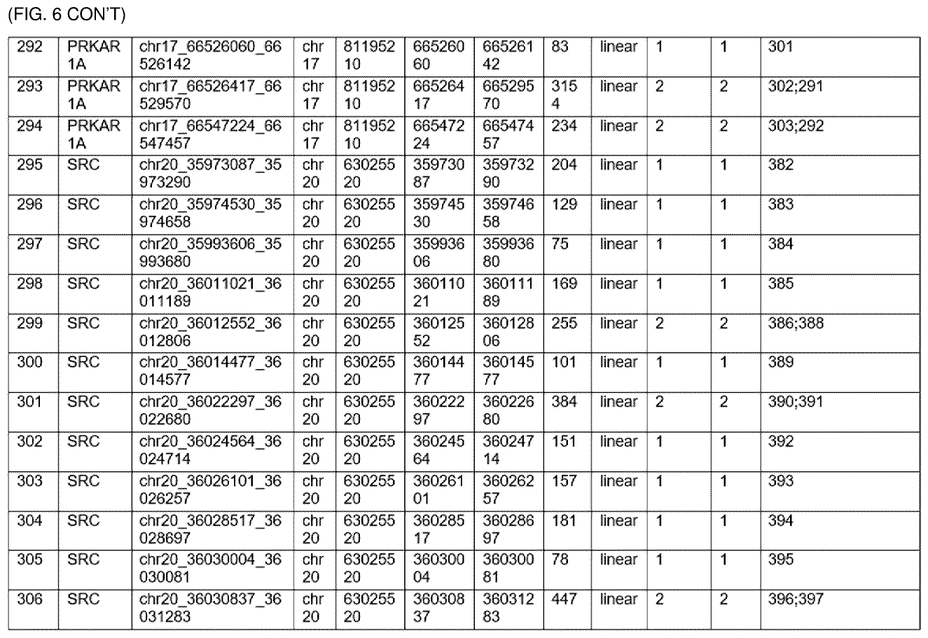

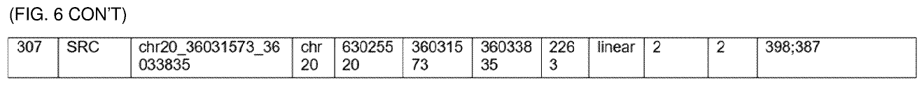

In order to validate our sequencing results, three of the SNVs located in the 3′UTR of RAPGEF3 (rs61917617, rs11168215 and rs11168214) found associated with ROP were selected for further analysis. PCR of the genomic DNA for a fragment of RAPGEF3 encompassing the location of the variants was performed on 19 samples including two samples from the discovery cohort, one ROP and one full-term, and 17 additional samples (11 ROP, 3 no ROP, and 3 full-term). The PCR reactions produced the expected amplicon of approximately 661 bp (FIG. 2A). PCR reactions were then digested with either SacII, Bsu36I, or BsaWI to detect the presence of respectively, rs61917617, rs11168215 or rs11168214 variants (FIG. 2A). Digestion with SacII produced the expected two fragments (a: ˜373 bp; b: ˜288 bp) in rs61917617 carriers. Digestion with BsaWI produced the expected two fragments 401 (fragment c: ˜401 bp; d: ˜260 bp) in rs11168214 carriers. Digestion with Bsu36I produced the expected two fragments (fragment e: ˜504 bp; f: ˜157 bp) in rs11168215 carriers. Complete digestions of the 661 bp amplicon were associated with homozygote carriers (Hom), partial digestions associated with heterozygote carriers (Het), and absence of digestion (uncut) was associated with non-carriers (−) of these variants (FIG. 2A). The three RAPGEF3 variants within the validation cohort perfectly correlated with each other (r²=1), further confirming their association to a single locus. The PCR results revealed that the genotypes of the two samples from the discovery cohort were exactly the same as their genotypes determined by the sequencing analysis, methodologically validating the discovery genotype. The allele frequencies of these three RAPGEF3 variants within the validation cohort was similar to their frequencies in the discovery cohort (FIG. 2B and TABL 2). However, the difference in the allele frequencies between the ROP and no ROP groups were not statistically significant (Chi squared, p=0.378). Finally, a combined analysis including the discovery and the validation cohorts for the above three RAPGEF3 variants revealed a significant difference in the allelic frequencies of the variants (Chi squared, p>0.039) between the ROP and no ROP groups (FIG. 2C). Odds ratios and 95% confidence intervals were 2.59 (1.12-6) for rs61917617 and rs11168215, and 2.4 (1.03-5.59) for rs11168214, similar to the results obtained with the discovery cohort (TABLE 2).

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Having thus described in detail preferred embodiments of the present invention, it is to be understood that the invention defined by the above paragraphs is not to be limited to particular details set forth in the above description as many apparent variations thereof are possible without departing from the spirit or scope of the present invention.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of detecting ADRβ pathway gene variants in a sample from a subject having or suspected of having a vascular retinopathy, the method comprising:
a. obtaining or having obtained a sample from the subject having or suspected of having a vascular retinopathy, the sample comprising a library of free nucleic acids comprising all or a portion of ADRβ pathway genes;
b. attaching at least one adapter to each free nucleic acid of the library of free nucleic acids, to generate adapter-ADRβ pathway nucleic acids wherein the at least one adapter comprises an amplification sequence;
c. amplifying the adapter-ADRβ pathway nucleic acids or nucleic acids derived from the adapter-ADRβ pathway nucleic acids using primers that bind to the amplification sequence, to generate ADRβ pathway gene amplicons; and
d. detecting whether ADRβ pathway gene variants are present in the ADRβ pathway gene amplicons.

2. The method of claim 1, wherein detecting whether ADRβ pathway gene variants are present in the ADRβ pathway gene amplicons further comprises sequencing at least a portion of the ADRβ pathway gene amplicons by DNA sequencing.

3. The method of claim 2, wherein at least one ADRβ pathway gene variant is detected.

4. The method of claim 1, wherein the method further comprises detecting that the subject has a vascular retinopathy.

5. The method of claim 1, wherein step (d) further comprises the following steps:
i. contacting the ADRβ pathway gene amplicons with a flow cell or solid surface comprising a plurality of oligonucleotides connected thereto to generate immobilized ADRβ pathway gene amplicons, wherein the oligonucleotides bind to the adapter;
ii. clonally amplifying the immobilized ADRβ pathway gene amplicons to generate clonally amplified ADRβ pathway gene amplicons;
iii. sequencing all of or at least a portion of the sequences of the clonally amplified ADRβ pathway gene amplicons from the one or more of the genes consisting of: RAPGEF3, ADCY7, ADCY9, PRKAR1A, or ADCY4.

6. The method of claim 5, wherein the gene variants in the ADRβ pathway genes are selected from the group consisting of: rs2240079, rs11168215, rs11168214, rs61917617, rs55683248, rs2072341, rs8082254, rs72847785, rs3181252, and rs17256902.

7. The method of claim 2 or 5, wherein the DNA sequencing method is massively parallel sequencing.

8. The method of claim 7, wherein the DNA sequencing method is targeted sequencing.

9. The method of claim 6, wherein
a. the ADRβ pathway gene variants rs8082254, rs72847785, rs3181254, and rs17256902 are protective alleles detected in a subject not having a vascular retinopathy; and
b. the ADRβ pathway gene variants rs2240079, rs11168215, rs61917617, rs11168214, rs55683248, and rs2072341 are risk factors detected in a subject having a vascular retinopathy.

10. The method of claim 4 or 9 wherein the vascular retinopathy is selected from the group consisting of: retinopathy of prematurity (ROP), proliferative diabetic retinopathy (PDR), a retinal vein occlusion retinopathy (RVO), hypertensive retinopathy, branch retinal vein occlusion (BRVO), central retinal artery occlusion (CRAO), central retinal vein occlusion, chronic BRVO, Coats' Disease, cotton-wool spots, dot and blot hemorrhage, flame-shaped hemorrhage, hard exudates, hollenhorst plaques, inferior hemicentral retinal vein occlusion, juxtafoveal telangiectasia, optociliary shunt, preretinal hemorrhage, retinal artery macroaneurysm, sickle proliferative retinopathy, subretinal hemorrhage, superior hemicentral retinal vein occlusion, fibrovascular proliferation in PDR, neovascularization of the disc, nonproliferative diabetic retinopathy (NPDR), panretinal photocoagulation, retinal vessel occlusive disease, macular degeneration including age-related macular degeneration, and any neovascular retinal disease.

11. The method of claim 10, wherein the vascular retinopathy is retinopathy of prematurity (ROP).

12. A method of preparing ADRβ pathway gene amplicons from a subject having or suspected of a vascular retinopathy, the method comprising:
a. obtaining or having obtained a sample from the subject having or suspected of having vascular retinopathy, the sample comprising free nucleic acids comprising all or a portion of ADRβ pathway genes;
b. attaching at least one adapter to the free nucleic acids, to obtain adapter-ADRβ pathway nucleic acids wherein the at least one adapter comprises an amplification sequence;
c. amplifying the adapter-ADRβ pathway nucleic acids or nucleic acids derived from the adapter-ADRβ pathway nucleic acids using primers that bind to the amplification sequence to generate ADRβ pathway gene amplicons.

13. A method for preparing an enriched nucleic acid sample useful for detecting an ADRβ pathway gene variant in a sample from a subject having or suspected of having a vascular retinopathy, the method comprising:
a. obtaining or having obtained a sample from the subject having or suspected of having vascular retinopathy, the sample comprising free nucleic acids comprising all or a portion of ADRβ pathway genes;
b. attaching at least one adapter to the free nucleic acids, to obtain adapter-ADRβ pathway nucleic acids wherein the at least one adapter comprises an amplification sequence; and
c. amplifying the adapter-ADRβ pathway nucleic acids or nucleic acids derived from the adapter-ADRβ pathway nucleic acids using primers that bind to the amplification sequence to generate ADRβ pathway gene amplicons.

14. The method of claim 12 or 13, further comprising the steps:
d. contacting the ADRβ pathway gene amplicons with a flow cell or surface comprising a plurality of oligonucleotides connected thereto, wherein the oligonucleotides are bound to the adapter; and
e. clonally amplifying of the ADRβ pathway gene amplicons to generate clonally amplified ADRβ pathway gene amplicons.

15. The method of claim 12 or 13, wherein the ADRβ pathway genes is selected from RAPGEF3, ADCY7, ADCY9, PRKAR1A, and ADCY4.

16. The method of claim 12 or 13, wherein the ADRβ pathway genes comprise RAPGEF3 and further wherein RAPGEF3 gene amplicons are obtained by using a forward primer having 80% homology or higher to that of the nucleotide sequence: 5′ CTTCCTTCATTTCTCCACCTG 3′ (SEQ ID NO: 1), and a reverse primer having 80% homology or higher to that of the nucleotide sequence 5′ TCTGTGTCCTCTTGCCTGC 3′ (SEQ ID NO: 2).

17. The method of claim 12 or 13, wherein the method additionally comprises the steps of end-repairing and dA-tailing before the attaching step (b), further wherein the end-repairing step excludes purifying the end-repaired products prior to the dA-tailing step and wherein the dA-tailing step excludes purifying the dA-tailing products.

18. The method of claim 12 or 13, wherein the free nucleic acids of the sample obtained in step (a) are not subjected to fragmentation.

19. The method of claim 12 or 13, wherein the sample comprising free nucleic acids obtained in step (a) is derived from saliva, blood, plasma, urine, cells, or tissue.

20. The method of claim 12 or 13, wherein the method further comprises detecting the clonally amplified ADRβ pathway gene amplicons by multiplex PCR.

21. The method of claim 14, wherein the method further comprises detecting the clonally amplified ADRβ pathway gene amplicons by massively parallel sequencing.

22. The method of claim 21, wherein at least one ADRβ pathway gene variant is detected.

23. The method of claim 22, wherein the ADRβ pathway gene variant is selected from the group consisting of: rs2240079, rs11168215, rs11168214, rs61917617, rs55683248, rs2072341, rs8082254, rs72847785, rs3181252, and rs17256902.

24. A method of treating a subject having or suspected of having a vascular retinopathy, the method comprising:
a. obtaining or having obtained a sample from the subject;
b. detecting or having detected one or a plurality of gene variants selected from: RAPGEF3: rs2240079, rs11168215, rs11168214 and rs61917617; ADCY7: rs55683248; ADCY9: rs2072341; PRKAR1A: rs8082254 and rs72847785; and ADCY4: rs3181252 and rs17256902 in the sample from the subject; and
c. administering a modulator of RAPGEF3, ADCY7, or ADCY9 to the subject having one or a plurality of gene variants selected from: RAPGEF3: rs2240079, rs11168215, rs11168214 and rs61917617; ADCY7: rs55683248; or ADCY9: rs2072341; or
administering a therapeutically effective amount of a modulator of PRKAR1A or ADCY4 to the subject having one or a plurality of gene variants of RAPGEF3: rs2240079, rs11168215, rs11168214 and rs61917617; ADCY7: rs55683248; or ADCY9: rs2072341; or
administering a modulator of PRKAR1A or ADCY4 to the subject having one or no protective allele from the gene variants selected from: PRKAR1A: rs8082254 and rs72847785; and ADCY4: rs3181252 and rs17256902.

25. A method of treating a subject having or suspected of having ROP by administering a modulator of a gene selected from: RAPGEF3, ADCY7, ADCY9, PRKAR1A or ADCY4, the method comprising:
a. determining or having determined if the subject has a mutation in one or more genes selected from: RAPGEF3, ADCY7, ADCY9, PRKAR1A or ADCY4; and
b. if the subject has a mutation in one or more genes selected from: RAPGEF3, ADCY7, or ADCY9, further determining whether the subject has one or a plurality of gene variants of RAPGEF3: rs2240079, rs11168215, rs11168214 and rs61917617; ADCY7: rs55683248; ADCY9: rs2072341 then administering to the subject a modulator of RAPGEF3, ADCY7, ADCY9, PRKAR1A, or ADCY4; or
c. if the subject has a mutation in one or more genes selected from: PRKAR1A or ADCY4, further determining whether the subject has one or a plurality of gene variants of PRKAR1A: rs8082254 and rs72847785 and ADCY4: rs3181252 and rs17256902 then administering to the subject a modulator of PRKAR1A or ADCY4 if the subject carries one or no protective gene variant.

26. A method of slowing vision loss or improving the vision in a subject having or suspected of having a vascular retinopathy, comprising administering to the subject a therapeutically effective amount of a modulator of one or more genes selected from: RAPGEF3, ADCY7, ADCY9, PRKAR1A or ADCY4.

27. The method of any of claims 24-26, wherein the modulator is a gene knockdown agent that is siRNA, shRNA, antisense RNA, or a gene knockout agent that is a transcription activator-like effector nuclease (TALEN) or a zinc finger nuclease (ZFN); or wherein the modulator is a gene activating agent that is a siRNA (short interfering RNA) or shRNA (sort hairpin RNA) molecule.

28. The method of any of claims 24-26, wherein the method further comprises administering one or more additional active agents or supportive therapies for treating, preventing, or reducing the severity of an eye disorder to the subject.

29. The method of claim 28, wherein the one or more supportive therapies is selected from the group consisting of: surgery, laser therapy, photocoagulation, anti-angiogenic therapy, vitrectomy, scleral buckle surgery, and pneumatic retinopexy, or any combination thereof.

30. The method of claim 28, wherein the one or more additional active agents is selected from the group consisting of: VEGF inhibitors, placental growth factor (PIGF) inhibitor, bevacizumab, ranibizumab, aflibercept, Ca²⁺ inhibitors, flunarizine, nifedipine, cryotherapy, hyperbaric oxygenation, Na⁺ channel blockers, topiramate, iGluR antagonists, (MK-801, dextromethorphan, eliprodil, flupirtine, antioxidants, dimethylthiourea, alpha-lipoic acid, superoxide dismutase, catalase, desferrioxamine, mannitol, allopurinol, calcium dobesilate, trimetazidine, EGB-761, anti-inflammatory agents, cyclodiathermy, cyclocryotherapy, ocular filtering procedures, implantation of drainage valves, antiplatelet therapy, aspirin, ticlopidine, clopidogrel, anticoagulant therapy, warfarin, heparin, steroids, systemic or local corticosteroids, prednisone triamcinolone, fluocinolone acetonide, dexamethasonc, steroid-sparing immunosuppressants, cyclosporine, azathioprine, cyclophosphamide, mycophenolate, mofetil, infliximab, etanercept, dietary supplements, vitamin C, vitamin E, lutein, zinc, folic acid, vitamin B6, vitamin B12, zeaxanthin, a VEGF and PIGF inhibitor, or any combination thereof.

31. The method of any of claims 24-26, wherein the RAPGEF3, ADCY7, ADCY9, ADCY4, or PRKAR1A modulator comprises a polypeptide capable of binding or sequestering RAPGEF3, ADCY7, ADCY9, ADCY4, or PRKAR1A.

32. The method of any of claims 12, 13, 24, 25, or 26, wherein the vascular retinopathy is selected from the group consisting of: retinopathy of prematurity (ROP), proliferative diabetic retinopathy (PDR), a retinal vein occlusion retinopathy (RVO), hypertensive retinopathy, branch retinal vein occlusion (BRVO), central retinal artery occlusion (CRAO), central retinal vein occlusion, chronic BRVO, Coats' Disease, cotton-wool spots, dot and blot hemorrhage, flame-shaped hemorrhage, hard exudates, hollenhorst plaques, inferior hemicentral retinal vein occlusion, juxtafoveal telangiectasia, optociliary shunt, preretinal hemorrhage, retinal artery macroaneurysm, sickle proliferative retinopathy, subretinal hemorrhage, superior hemicentral retinal vein occlusion, fibrovascular proliferation in PDR, neovascularization of the disc, nonproliferative diabetic retinopathy (NPDR), panretinal photocoagulation, retinal vessel occlusive disease, macular degeneration including age-related macular degeneration, and any neovascular retinal disease.

33. A method of screening for a gene knockout or gene knockdown treatment, or a gene knock-in or gene activation treatment of a retinopathy, the method comprising performing PCR for gene variants of one or more genes selected from: RAPGEF3: rs2240079, rs11168215, rs11168214 and rs61917617; ADCY7: rs55683248; and ADCY9: rs2072341 in a mammalian animal model of oxygen-induced retinopathy, then administering the gene knockdown or gene knockout treatment, or gene knock-in or gene activation treatment, to the animal model, then observing the knockdown or knockout effect.

34. A method of screening for a gene knock-in or gene activation treatment, or a gene knockout or gene knockdown treatment, the method comprising performing PCR for gene variants selected from one or more of: PRKAR1A: rs8082254 and rs72847785, and ADCY4: rs3181252 and rs17256902 in a mammalian animal model of oxygen-induced retinopathy, then administering the gene knock-in or gene activation treatment, or a gene knockout or gene knockdown treatment, to the animal model, then observing the knock-in or gene activation effect.


SEQUENCE LISTING (2 sequences)

SEQ ID NO: 1 - DNA, 21 bp, synthetic construct
  source 1..21: mol_type = other DNA
cttccttcatttctccacctg

SEQ ID NO: 2 - DNA, 19 bp, synthetic construct
  source 1..19: mol_type = other DNA
tctgtgtcctcttgcctgc